(19) United States
(12) Patent Application Publication
(10) Pub. No.: US 2012/0177608 A1
(43) Pub. Date: Jul. 12, 2012

(54) PSEUDOMONAS AERUGINOSA BACTERIOPHAGE(S) AND USES THEREOF

(75) Inventors: Paul Ross, Co. Cork (IE); Debebe Alemayehu, Co. Cork (IE); Colin Hill, Cork (IE); James Gerard Martin, Montreal (CA)

(73) Assignees: AGRICULTURE AND FOOD DEVELOPMENT AUTHORITY (TEAGASC), Co. Carlow (IE); UNIVERSITY COLLEGE CORK, NATIONAL UNIVERSITY OF IRELAND, CORK, Co. Cork (IE)

(21) Appl. No.: 13/330,744

(22) Filed: Dec. 20, 2011

(30) Foreign Application Priority Data
Dec. 20, 2010 (EP) 10195995.5

(51) Int. Cl. A61K 35/76; A61P 31/04; C12N 7/00

(52) U.S. Cl. 424/93.6; 435/235.1

(57) ABSTRACT

An isolated bacteriophage MR299-2 or NH-4 deposited under NCIMB Deposit Accession Nos. 41729 and 41730, respectively, is described. The phages have lytic activity against P. aeruginosa strains, including mucoid and CF clinical isolate strains. Variant hages are also described, wherein said variants retain the phenotypic characteristics of said phage and wherein said phage and variants thereof have lytic activity against P. aeruginosa strains.

### CROSS REFERENCE TO RELATED APPLICATIONS

This application claims benefit of European Patent Application No.10195995.5 filed Dec. 20, 2010, the contents of which are incorporated herein by reference in its entirety.

1. Field of the Invention

The present invention relates to isolated bacteriophages (phages) having lytic activity against P. aeruginosa strains, and the use of the isolated phages for the treatment of a P. aeruginosa mediated infection, for example in a mammal with cystic fibrosis. In particular, the invention relates to a cocktail of phages capable of clearing P. aeruginosa infection from the lung of a mammal. The invention also relates to a pharmaceutical composition for the treatment of an individual suffering from a P. aeruginosa infection.

2. Background to the Invention

Cystic fibrosis (CF) is a common inherited chronic genetic disorder that affects mainly the lungs and digestive system (pancreas and intestine) of children and adults worldwide. CF also affects the mucus and sweat glands of the liver, sinuses, and sex organs causing progressive disability due to multisystem failure. CF is caused by mutations in the CF transmembrane conductance regulator (Cftr) gene. The defective gene and its protein product cause the body to produce unusually thick and sticky mucus and improper amounts of chloride-containing secretions into ducts and body cavities. This secretion clogs the lung, leading to life-threatening lung infections.

Lungs of CF patients are often colonised or infected in infancy and early childhood with P. aeruginosa that may damage the epithelial surface, resulting in altered airway physiology and impairment of mucocilliary clearance. Such chronic infection of lungs with P. aeruginosa is the main proven cause of lung function decline and ultimate mortality in CF patients. In fact, 80 to 95% of patients with CF succumb to respiratory failure brought by chronic bacterial infection and concomitant airway inflammation. Current evidence suggests one way in which P. aeruginosa persists in CF lungs is due to its ability to form biofilms in the lungs of patients. Another significant factor is the inherent resistance of P. aeruginosa to many antibiotics due to the production of exopolysaccharide. As a result the most prevalent and severe chronic lung infection in CF patients is caused by mucoid P. aeruginosa. Reports have shown that organisms in biofilms are able to tolerate 10-1000 fold higher antibiotics than planktonic bacteria and this often makes the antibiotic concentration needed to eradicate the biofilm above the peak serum concentration of antibiotic, rendering it ineffective in treating biofilm associated infections. The continued emergence and re-emergence of Pseudomonas pathogens which are resistant to one or more chemical antibiotics and have the ability to form biofilms poses a continuous challenge in the treatment of lung infections in CF patients. As a result, there is an urgent need for an alternative, non-antibiotic approach such as phage therapy.

A number of studies have investigated the development and differentiation of Pseudomonas biofilms in vitro using a variety of static and flow cell assays. Recently Anderson et al., (2008) demonstrated a tissue culture model to grow GFP-tagged P. aeruginosa biofilms on CF derived human airway cells that promotes the formation of highly antibiotic resistant microcolonies (biofilms). A very recent study by Debarbieux et al. (2010) reported that the amount of light measured in lux-tagged Pseudomonas PAK strain-infected mice decreased to a minimum level in 6 h when phage PAK-P1 was administered 2 h after infection. However, there is very little information and understanding regarding the efficacy of phages in clearing Pseudomonas biofilms growing on CF lung tissue.

Bacteriophages (or phages) are viruses consisting of an outer protein capsid enclosing genetic material (single-stranded RNA, double stranded RNA, single stranded DNA, or double stranded DNA) in either a circular or linear arrangement, capable of infecting a bacterial cell, and may cause lysis to its host cell. Phages may also exist as pro-phages, which are phages that exist in a dormant state or which are even defective. In this case the genome of the phage is either integrated into that of the host bacterium, or is replicated autonomously.

Currently, there is a lack of information and understanding about the effect of phages on biofilms growing on lung tissue models. It is an object of the invention to overcome at least one of the above-referenced problems.

### SUMMARY OF THE INVENTION

Lungs of CF patients are often colonised or infected in infancy and early childhood with organisms that may damage the epithelial surface, resulting in altered airway function and impairment of mucocilliary clearance. Such functional impairment to the lungs leads to increased risk for P. aeruginosa infection in CF patients. Biofilm formation by P. aeruginosa is considered an important factor as to why it is becoming increasingly difficult to treat CF lung infections using antibiotics. The invention is based on the discovery of phages that have lytic activity against a broad range of Pseudomonas isolates, including mucoid Pseudomonas and CF clinical isolates. This invention is also based on the surprising discovery of the clearance of Pseudomonas by a cocktail of phages from lungs of infected mice and biofilms growing on the surfaces of a Cystic Fibrosis Bronchial Epithelial (CFBE41o-) monolayer. p16Slux-tagged P. aeruginosa was used to monitor lung infection in mice and biofilm growth. A cocktail of phages were used to kill Pseudomonas in both in vivo and in vitro systems. The data presented shows that a phage cocktail of the invention can clear P. aeruginosa effectively from lungs of infected mice and from biofilms growing on CF epithelial cell monolayer.

Accordingly, in a first aspect, the invention relates to an isolated phage MR299-2 or phage NH-4 deposited under NCIMB Deposit Accession Nos. 41729 and 41730, respectively, and variants or progeny thereof.

Phages of the invention have been found to have lytic activity against a broad spectrum of P. aeruginosa strains, including CF clinical isolates and mucoid strains. The phages have shown utility in clearing P. aeruginosa effectively from lungs of infected mice and from biofilms growing on CF epithelial cell monolayer.

Suitably, isolated phage MR299-2, and variants thereof, have lytic activity against at least P. aeruginosa strains MR299 and NH57388A. Ideally, the isolated phage MR299-2, and/or variants thereof, have lytic activity against at least P. aeruginosa isolates CH001, MR299, MR325, MR326, MR327, MR330, MR331, POA1 MR299 and NH57388A (the characteristics of which are described in Table 6).

Typically, isolated phage NH-4, and/or variants thereof, have lytic activity against at least P. aeruginosa strains MR299 and NH57388A. Ideally, the isolated phage NH-4, and/or variants thereof, have lytic activity against at least P. aeruginosa isolates CH001, MR299, MR300, MR326, MR327, MR330, MR331, POA1 MR299 and NH57388A (the characteristics of which are described in Table 6).

Generally, variants of phage MR299-2 have at least >95%, 96%, 97%, 98% or 99% sequence identity to MR299-2 (SEQUENCE ID NO: 5). Typically, the variants will have a MW from 40 kD to 50 kD, suitably from 42 kD to 47 kD, and ideally from 44 kD to 46 kD. Ideally, a genome of the variant has at least 1, 2, or 3 open reading frames (ORF's). Suitably, the variant exhibits lytic activity towards Pseudomonas isolates MR299 (NCIMB Deposit Accession No. 41731) and NH57388A (Hoffmann et al. Antimicrob Agents Chemother. 2007 October; 51(10): 3677-3687).

Generally, variants of phage NH-4 have at least >95%, 96%, 97%, 98% or 99% sequence identity with phage NH-4 (SEQUENCE ID NO: 6). Typically, the variants will have a MW from 60 kD to 70 kD, suitably from 64 kD to 68 kD, and ideally from 66 kD to 67 kD. Preferably, a genome of the variant has at least 3, 4, 5, 6, 7 or 8, ideally 8, unique open reading frames (ORF's). Suitably, the variant exhibits lytic activity towards Pseudomonas isolates MR299 (NCIMB Deposit Accession No. 41731) and NH57388A (Hoffmann et al. Antimicrob Agents Chemother. 2007 October; 51(10): 3677-3687).

The invention also provides isolated progeny of the phages MR299-2 or NH-4, wherein said progeny exhibits lytic activity towards Pseudomonas isolates MR299 (NCIMB Deposit Accession No. 41731) and NH57388A (Hoffmann et al. Antimicrob Agents Chemother. 2007 October; 51(10): 3677-3687).

Suitably, the isolated progeny have a RFLP profile that is substantially equivalent to a RFLP profile of the phage when prepared using the techniques described below.

In a second aspect, the invention provides a phage cocktail comprising (a) an isolated phage MR299-2 of the invention, or an isolated variant or progeny thereof, and (b) an isolated phage NH-4 of the invention, or an isolated variant or progeny thereof. Typically, the phage cocktail is capable of clearing P. aeruginosa infection from lungs of infected mice. Suitably, the phage cocktail is capable of clearing P. aeruginosa infection from biofilms growing on the surfaces of a Cystic Fibrosis Bronchial Epithelial (CFBE41o-) monolayer.

In a third aspect, the invention provides a pharmaceutical preparation comprising an isolated phage of the invention, or an isolated variant or progeny thereof, and a pharmaceutically acceptable carrier.

In a fourth aspect, the invention provides a pharmaceutical preparation comprising a phage cocktail of the invention and a pharmaceutically acceptable carrier.

In a fifth aspect, the invention provides an isolated phage of the invention, or an isolated variant or progeny thereof, or a phage cocktail of the invention, for use in the treatment or prevention of P. aeuriginosa-mediated infection, especially a lung infection, preferably a chronic lung infection, in a mammal, typically a mammal with an underlying disease or condition which renders them prone to contracting a P. aeuriginosa-mediated infection, for example a mammal with cystic fibrosis (CF). Thus, the phage of the invention, or variants or progeny thereof, or the phage cocktail of the invention, may be employed to prevent or treat CF in a mammal. In a preferred embodiment, the use of the invention involves administering the phage, or phage cocktail, of the invention to the mammal within 12, 10, 8, 6, 4, 2 or 1 hours of the establishment of the infection.

Thus, the invention relates to the use of the isolated phage, or phage cocktail, of the invention in the treatment or prevention of an acute or chronic P. aeuriginosa-mediated infection in a mammal. The invention relates to the use of the isolated phage, or phage cocktail, of the invention in the treatment of cystic fibrosis in a mammal.

The DNA sequence of phage MR299-2 is provided in SEQUENCE ID NO: 5. The invention also relates to an isolated DNA sequence having at least >95% sequence identity with SEQUENCE ID NO: 5. The invention also relates to an isolated DNA sequence having at least 98%, 98.5%, 98.6%, 98.7%, 98.8%, 98.9%, 99%, 99.1%, 99.2%, 99.3%, 99.4%, 99.5%, 99.6%, 99.7%, 99.8% or 99.9% sequence identity with SEQUENCE ID NO: 5. The invention also relates to a phage encoded by an isolated DNA sequence of the invention.

The DNA sequence of phage NH-4 is provided in SEQUENCE ID NO: 6. The invention also relates to an isolated DNA sequence having at least >95% sequence identity with SEQUENCE ID NO: 6. The invention also relates to an isolated DNA sequence having at least 98%, 98.5%, 98.6%, 98.7%, 98.8%, 98.9%, 99%, 99.1%, 99.2%, 99.3%, 99.4%, 99.5%, 99.6%, 99.7%, 99.8% or 99.9% sequence identity with SEQUENCE ID NO: 6. The invention also relates to a phage encoded by an isolated DNA sequence of the invention.

### Definitions

The phages of the invention should be taken to mean P. aeruginosa phage MR299-2 and NH-4 deposited with NCIMB, an international depositary authority recognized for the purposes of patent procedure under the Budapest Treaty, on 24 Jun. 2010, and receiving NCIMB Deposit Accession Nos. 41729 and 41730, respectively. Said phages have lytic activity against P. aeruginosa strains MR299-2 and NH-4 (NH57388A). The phages of the invention also include variants of MR299-2 and NH-4 (as defined below) which have lytic activity against the same P. aeruginosa strains.

In the specification, the term “lytic activity” should be considered to mean as having activity against a target bacteria as determined by a plaque assay. An example of a plaque assay is one where phages are purified by successive single plaque isolation and propagation using Luria Broth double layer agar plates. In general, a single plaque is picked from a plate using a sterile capillary tube and added into mid log-phase Pseudomonas culture (108 (colony forming units) cfu/ml) supplemented with 10 mM CaCl2. The culture phage mixture is incubated at 37 oC overnight. The lysate is filter-sterilised through a 0.45-μm pore size sterile filter, serial dilutions are made and plagued on a lawn of host culture. Single plaque isolation and plaquing process is repeated three additional times after which purified phages are obtained. Phage titer is determined as plaque forming units (pfu/ml) by plaque assay as previously described (O'Sullivan et al. 2001). Those phages having lytic activity against the bacteria of interest are indicated by a clear halo in the double layer agar plates (see, for example, FIG. 15).

Specifically, the term “variant” as applied to phage MR299-2 means a phage that exhibits lytic activity towards Pseudomonas isolates MR299 (NCIMB Deposit Accession No. 41731) and NH57388A (Hoffmann et al. Antimicrob Agents Chemother. 2007 October; 51(10): 3677-3687) and whose genome has at least 90% sequence identity to the genome of phage MR299-2 (SEQUENCE ID NO: 5). Typically, a variant of phage MR299-2 has at least 91%, 92%, 93%, 94%, 95%, 96%, 97%, 98% or 99% sequence identity to phage MR299-2 (SEQUENCE ID NO: 5). Preferably, the variant has at least 1, 2 or 3, ideally 3, unique open reading frames (ORF's). The term should be understood to include genetically modified versions of the deposited phage in which the genetic code is manipulated by means of, for example, genetic engineering or serial passage.

Specifically, the term “variant” as applied to phage NH-4 means a phage that exhibits lytic activity towards Pseudomonas isolates MR299 (NCIMB Deposit Accession No. 41731) and NH57388A (Hoffmann et al. Antimicrob Agents Chemother. 2007 October; 51(10): 3677-3687) and whose genome has at least 90% sequence identity to the genome of phage NH-4 (SEQUENCE ID NO: 6). Typically, a variant of phage NH-4 has at least 91%, 92%, 93%, 94%, 95%, 96%, 97%, 98% or 99% sequence identity to phage NH-4 (SEQUENCE ID NO: 6). Preferably, the variant has at least 3, 4, 5, 6, 7 or 8, ideally 8, unique open reading frames (ORF's). The term should be understood to include genetically modified versions of the deposited phage in which the genetic code is manipulated by means of, for example, genetic engineering or serial passage.

In the specification, the term “Unique Open Reading Frames (ORF's)” should be understood to mean a gene showing no homologous match in another organism's genome during a protein and/or nucleotide sequence alignment search in a database. In this specification, the term “sequence identity” should be understood to mean the % of aligned residues in a test sequence that are identical to the corresponding residues in the reference sequence. In this context, a phage genome that shares 99% nucleic acid sequence identity with the genome of phage MR299-2 (SEQ ID NO: 5) is one in which any 99% of aligned nucleic acid residues are identical to the corresponding residues in SEQ ID NO: 5. Typically, a protein and/or nucleotide sequence alignment search or comparison is carried out using the web-based Basic Local Alignment Search Tool (BLAST) hosted by the National Centre for Biotechnology Information (NCBI) (www.ncbi.nlm.nih.gov/BLAST.cgi).

Pseudomonas strain MR299 was deposited with the NCIMB, an international depositary authority recognized for the purposes of patent procedure under the Budapest Treaty, on 24 Jun. 2010, and receiving NCIMB Deposit Accession Nos. 41731. Pseudomonas strain NH4 refers to a stable mucoid Cystic Fibrosis sputum isolate NH57388A isolated from a chronically colonized CF patient attending the Danish CF Center, Rigshospitalet, Copenhagen, Denmark (N. Hoffmann, B. Lee, M. Hentzer, T. B. Rasmussen, Z. Song, H. K. Johansen, M. Givskov, and N. Høiby “Azithromycin Blocks Quorum Sensing and Alginate Polymer Formation and Increases the Sensitivity to Serum and Stationary-Growth-Phase Killing of P. aeruginosa and Attenuates Chronic P. aeruginosa Lung Infection in Cftr^{-/-} Mice” Antimicrob Agents Chemother (2007) 51(10): 3677-3687).

In the specification, the term “isolated” should be considered to mean material removed from its original environment in which it naturally occurs, for example, in this instance bacteriophage specific for a particular bacterium. The removed material is cultivated, purified and cultured separately from the environment in which it was located. Thus, the purified isolated phage in this instance does not contain any significant amounts of other phages. For the term “progeny” or “isolated progeny”, the term should be considered to mean replicates of the original phage, including descendents of the phage created by serial passage of the isolated phage or by other means known in the art, that are typically isolated in the same manner as described above, and/or phages having a substantially equivalent RFLP profile to the deposited phage. In the specification, the term “Restriction Fragment Length Polymorphism profile” or “RFLP profile” should be considered to mean the EcoR1 restriction digestion profile of FIG. 5. The term “RFLP profile that is substantially equivalent” should be considered to mean a RFLP profile describing acceptable variability between genomes of identical propagated organisms of isolated progeny as described by Tenover et al.

In the specification, the term “phage cocktail” should be considered to mean a combination comprising the two phages of the invention, or variants or progeny thereof, each of which have been isolated from the environment from which they were originally found or have been produced by means of a technical process such as genetic engineering or serial passage techniques.

“Treating” (or “treat”) as used herein includes its generally accepted meaning which encompasses prohibiting, preventing, restraining, and slowing, stopping or reversing progression, severity, of a cause or resultant symptom of a P. aeruginosa infection. The term includes causal or symptomatic treatment. As such, the methods of this invention encompass both therapeutic and prophylactic administration.

In this specification, the term “prevention” should be taken to mean inhibition or prevention of the growth of P. aeruginosa bacteria, typically P. aeruginosa biofilms, ideally in the lungs of an individual suffering from CF.

In the specification, the term “mammal” or “individual” as employed herein should be taken to mean a human; however it should also include higher mammals for which the prophylaxis, therapy or use of the invention is practicable.

In this specification, the term “administering” should be taken to include any form of delivery that is capable of delivering the phage to a site of infection, including local delivery, intravenous delivery, oral delivery, intranasal delivery, intramuscular delivery, intrathecal delivery, transdermal delivery, inhaled delivery and topical delivery. Methods for achieving these means of delivery will be well known to those skilled in the art of drug delivery. For treatment or prophylaxis of lung infections, especially chronic P. aeruginosa infections in patients with compromised lung function, such as CF patients, pulmonary delivery is ideal as it delivers the phages of the invention directly to the small airways of the lung where the target bacteria mucoid infections exist.

In this specification, the term “pharmaceutical composition” should be taken to mean compositions comprising a therapeutically effective amount of a phage, and a pharmaceutically acceptable carrier or diluent. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the phage is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like.

The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the therapeutic, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

“Effective amount” refers to the amount or dose of the phage, upon single or multiple dose administration to the patient, which provides the desired effect in the patient under treatment. An effective amount can be readily determined by the attending diagnostician, as one skilled in the art, by the use of known techniques and by observing results obtained under analogous circumstances. In determining the effective amount or dose of phage administered, a number of factors are considered by the attending diagnostician, including, but not limited to: the species of mammal; its size, age, and general health; the specific disease involved; the degree of or involvement or the severity of the disease; the response of the individual patient; the particular bacteriophage or combination of bacteriophages administered; the mode of administration; the bioavailability characteristics of the preparation administered; the dose regimen selected; the use of concomitant medication; and other relevant circumstances. For example, the phages of the invention may be administered at a concentration of about 10⁵-10¹¹ PFU/ml.

In the specification the terms “comprise, comprises, comprised and comprising” or any variation thereof and the terms “include, includes, included and including” or any variation thereof are considered to be totally interchangeable and they should all be afforded the widest possible interpretation and vice versa.

### BRIEF DESCRIPTION OF THE DRAWINGS

The invention will be more clearly understood from the following description of an embodiment thereof, given by way of example only, with reference to the accompanying drawings, in which:—

FIG. 1 Confluent CFBE41o-cell mono-layer (8-10 days old), A and 24 hr old Pseudomonas micro-colonies (biofilms) scattered on surface of CFBE41o-cell monolayer, B.

FIG. 2 Growth of lux-tagged Pseudomonas biofilms on surface of CFBE41o-cell monolayer. Light measured over the 24 hr incubation period.

FIG. 3 Light (photon unit) measurements of biofilm growth on CFBE41o-monolayer (data from FIG. 2). Light reading was taken at times 1, 5 and 24 hr. A and B show respectively light readings before and after washing biofilm culture with MEM medium. values are mean±S.D readings from 6 wells.

FIG. 4 Fluorescent image of 24 hr old CF P. aeruginosa biofilms formed on CFBE41o-cell monolayer following calcofluor (fluorescent enhancer) staining. A and B showing matured biofilms of strains NH57388A and MR299 respectively. C and D showing weakened and opened biofilms of strains NH57388A and MR299 respectively after exposed to phage mix for 22-24 hr.

FIG. 5 EcoR1 restriction digestion profile of phage DNA. Lane 1, Hyperladder I marker, Lanes 2 and 3 phages φMR299 and φNH-4 respectively

FIG. 6 Electron Microscopy Images of the podophage φMR229-2, (A) and myophage φNH-4, (B & C), stained with phosphotungstic acid 0.2%. Arrow in A, showing a short tail of 10-20 nm long of the podophage, and B and C showing contracted, B and relaxed tail sheath, C of the myophage.

FIG. 7 Pseudomonas biofilm clearing by phage mix. A, Biofilms of non-mucoid MR299 strain and B, mucoid NH57388A strain grown on CFBE41o-cell monolayer for 24 hr. Cultures washed with MEM medium to remove planktonic cells and undefined phage mix applied and incubated for 22-24 hr. Light was monitored and reading taken at times indicated. Wells in the top row (a) control biofilm cultures without phage added and bottom row (b), with phage added.

FIG. 8 Clearing of Pseudomonas biofilms on CFBE41o-monolayer by crude phage mix (data from FIG. 7). Phages were was applied to a 24 hr biofilm of NH57388A and MR299 cells and light monitored over 22-24 hr period. Values are mean±S.D readings from 3 wells.

FIG. 9 Pseudomonas biofilm clearing by phage mix. Pseudomonas non-mucoid MR299 strain biofilm culture, A and mucoid NH57388A strain culture, B. Undefined phage mix was added to the top row wells, a, and defined mix of phage φNH-4 and φMR299-2 added to bottom wells, b. Light was monitored at time 0 and 24 hr.

FIG. 10 Phage titer estimated at time 0 and 24 hr after addition of phage into Pseudomonas biofilm of MR299, A and NH57388A strains, B. values are mean±S.D readings from 3 wells.

FIG. 11 Clearing of Pseudomonas MR299 strain infection from lungs of infected mice by a cocktail of φNH-4 and φMR299-2 phages. Top row, control mice and bottom row, phage treated mice.

FIG. 12 Clearing of Pseudomonas NH57388A strain infection from lungs of infected mice by a cocktail of φNH-4 and φMR299-2 phages. Top row, control mice and bottom row, phage treated mice.

FIG. 13 PCR reactions (16S rDNA) of CF Pseudomonas isolates. A, genera specific primers product (618 bp) and B, species specific primer product (956 bp). Lanes 1-9, CF Pseudomonas isolates, Lane 10, P. aeruginosa POA1 (control). M, hyperLadder IV DNA marker.

FIG. 14 Colony morphology of P. aeruginosa overnight cultures on Luria Broth (LB) agar plate of the mucoid strain NH57388A, A and a non-mucoid strain MR299, B.

FIG. 15 Plaques formed by phage MR 299-2 on P. aeruginosa lawn growing on Luria Broth (LB) Double layer agar plate (lytic activity test).

### DETAILED DESCRIPTION OF THE DRAWINGS

### Bacterial Strains and Culture Conditions

CF Pseudomonas strains used in this study are shown (Table 1). LB medium was used throughout this study for the culturing of Pseudomonas strains. A standard system for identification was performed to determine metabolism and assimilation profile of Pseudomonas isolates using API kit (API 20 NE, Bio-Mereux, Etoile, France). API reactions were read according to the reading table and identification was obtained by referring to the analytical profile index provided. For phage isolation, a double-strength LB medium was made by doubling the weight of dry ingredients required to prepare single-strength LB broth. Cultures were grown at 37° C. under aerobic conditions and shaking at 180 rpm. Solid media and soft agar overlays contained 1.5% and 0.7% agar (BD Difco, Oxford, UK), respectively.

Transformation of Pseudomonas with p16lux Plasmid

P. aeruginosa strains NH57388A (mucoid) and MR299 (non-mucoid) were transformed with p16Slux plasmid (Reidel et al. 2007) by the method of Shen et al., (2006). In brief, Pseudomonas strains were grown in LB medium at 25° C. until O.D 0.8 (600nm) is reached. To facilitate electroporation, Pseudomonas exopolysaccharide was digested by adding Aliginate-lyase (Sigma Cat. No. A1603, Sigma, Japan) to a final concentration of 2 U ml⁻¹. The cell enzyme mixture was incubated at 37° C. for 30 min. Cells were centrifuged at 10,000×g (4° C.) for 10 min. The resultant pellet was washed twice with chilled electroporation buffer (containing 300 mM glucose, 5 mm CaCl₂ and 25 mM HEPES in distilled water, pH 7.0) and resuspended in 0.1 ml of buffer (1×10⁹⁻¹⁰ cfu/ml). These electro-competent cells were mixed with 10 μl of p16Slux tagged plasmid DNA (Mug) and incubated on ice for 10-15 min. The mixture was immediately transferred to a chilled electroporation cuvette (0.2 cm electrode gap Gene-pulser cuvette, BioRad, Hercules, Calif., USA) and subjected to a single Voltage shock by applying a pulse (settings: capacitor, 25 μF; resistor, 200Ω and voltage, 2.5 kV on ECM 630, BTX, Harvard precision pulse apparatus, Holliston, Mass., USA). Immediately after electric shock, 900 μl chilled SOC medium was added to the mixture and incubated on ice for 10 min, followed by incubation at 30° C. for 2-3 hr. Cells were concentrated by centrifugation at 10,000×g (room temperature) and transformants were obtained by plating cells on LB agar containing erythromycin (800 μg) and incubating at permissive temperature (30° C.) for 24-48 hr. Ery^{r} colonies were checked for light emission using a VivoVision WIS100 imaging system (Xenogen, Alameda, Calif.), luminescence was measured in relative light units (RLU, in photons s⁻¹) and the presence of p16Slux was confirmed by mini-prep and restriction analyses.

### Identification of Pseudomonas Using PCR Reactions

Colony PCR was performed using genus-specific primers PA-GS-F (5′-GACGGGTGAGTAATGCCTA-3′(SEQ ID No. 1)) and PA-GS-R (5′-CACTGGTGTTCCTTCCTATA-3′(SEQ ID No. 2)), designed to the 16S rDNA sequences of Pseudomonas. Positive isolates were further analyzed and verified by P. aeruginosa species-specific primers PA-SS-F (5′-GGGGGATCTTCGGACCTCA-3′ (SEQ ID No. 3)) and PA-SS-R (5′-TCCTTAGAGTGCCCACCCG-3′(SEQ ID No. 4)). All primers and PCR conditions were according to Spilker et al., (2004).

### Isolation of Phages From Sewage

Pseudomonas phages were isolated from fresh sewage obtained from a local treatment plant as described previously (Alemayehu et al., 2009) with some modifications. Sewage samples were centrifuged at 3,200×g value (Heraeus Labofuge 400 Centrifuge, Thermo Fisher Scientific, Inc. MA, USA) for 12 min and the supernatant filtered using a 0.45 μm pore size filter (Sarstedt, Actiengeselischaft & Co., Germany). The sewage filtrate was mixed with an equal volume of double strength LB broth, supplemented with 10 mM CaCl₂ and inoculated with a mixture of CF P. aeruginosa cultures. Samples were incubated overnight aerobically (37° C.) with slow shaking (20-30 rpm). Following overnight incubation, cultures were centrifuged at 3,200×g value for 12 minutes to remove bacterial cells and debris and the supernatant was filtered through sterile 0.45-μm pore size filter. A double layer LB agar plate containing a lawn of host cultures and 10 mM CaCl₂ was prepared and 10 μl of cell free filtrate containing phage applied. Plates were examined for presence of plaques after incubating aerobically for 18-24 hr at 37° C.

### Plaque Purification and Phage Titering

Phages were purified by successive single plaque isolation and propagation. In general, a single plaque was picked from a plate using a sterile capillary tube and added into mid log-phase Pseudomonas culture (10⁸ cfu/ml) supplemented with 10 mM CaCl₂. The culture phage mixture was incubated at 37° C. overnight. The lysate was filter sterilised through a 0.45-μm pore size sterile filter, serial dilutions were made and plagued on a lawn of host culture. Single plaque isolation and plaquing process was repeated three additional times after which purified phages were obtained. Phage titer was determined as plaque forming units (pfu/ml) by plaque assay as previously described (O'Sullivan et al. 2001).

### DNA Extraction and Restriction Digestion Analysis

High titer purified phage suspension was prepared by concentration of phage particles from 400 ml cell lysate in LB medium to a final volume of 1 ml in sterile ice-cold ammonium acetate (0.1 M, pH 7.2) according to Capra et al., (2006) and Alemayehu et al. (2009). DNA was extracted from high titer purified phage according to Moineau et al., (1994) and Alemayehu et al., (2009). Phage DNA was digested with restriction endonuclease EcoRI (New England Biolabs, MA, USA) according to the supplier's recommendation and digested samples were analysed by gel electrophoresis using agarose gel (0.7%) containing ethidium bromide.

### Electron Microscopy and Phage Characterization

For electron microscopy, a drop of high titer phage suspension (1-2×10⁹ pfu/ml) was deposited on carbon-coated copper grids, negatively stained with 2% (wt/vol) potassium phosphotungstate (pH 7.2) and examined with Zeiss Supra 40VP scanning electron microscope (Carl Zeiss SMT LTD Cambridge, UK) fitted with Scanning Transmission Electron Microscope Detector (STEM) operating in bright field mode at 25 kV accelerating voltage (National Food Imaging Centre, MFRC).

### Cell Culture and Pseudomonas Biofilm Formation on Surface of Human Bronchial Epithelial Cells (CFBE41o-)

Cystic Fibrosis Bronchial Epithelial (CFBE41o-) cell cultures (Bruscia et al., 2002, Cozens et al., 1994) were grown according to Anderson et al. (2008). In general, CFBE41o-cells were seeded in sterile 6-well, flat bottom tissue culture plates (Sarstedt, Newton, N.C., USA) at a concentration of 10⁶ cells/well and maintained in minimal essential medium (MEM) containing 10% fetal bovine serum, 2 mM L-glutamate, 100 ug/ml penicillin and 100 ug/ml streptomycin (all from Invitrogen GIBCO, Invitrgen, UK). The cells were grown at 37° C. and 5% CO₂ using Jouan cell life, Incubator (Jouan, IGO150, St. Herblain, France) for 8-10 days until cells formed a confluent monolayer and tight junctions. MEM medium was changed every 2-3 days until confluent growth was achieved.

For biofilm formation, lux-tagged P. aeruginosa cells were grown on the confluent CFBE41o-monolayer using a co-culture model system (Anderson et al., 2008). Once the monolayer growth was achieved (between 8-10 days), the medium was replaced with 1.5 ml fresh MEM (without fetal bovine serum, penicillin and streptomycin) and lux tagged Pseudomonas cells inoculated (1-2×10⁷ cfu/well). Plates were incubated at 37° C. and 5% CO₂ for 1 hr. Then, the medium containing the unattached (planktonic) Pseudomonas cells was removed using sterile serological pipette and replaced with fresh MEM supplemented with 0.4% arginine and incubated further for 24 hr. Then, planktonic Pseudomonas cells were removed and the biofilm culture washed twice using MEM supplemented with 0.4% arginine. Epithelial-monolayer integrity and growing Pseudomonas microcolonies were assessed by phase-contrast microscopy (Olympus IX50, inverted system microscope, Olympus Co. Tokyo, Japan). Luminescence from biofilms was monitored by VivoVision IVIS100 imaging system (Xenogen, Alameda, Calif., USA). Biofilm cfu estimation was according to Wirtanen et al., (2001) with some modifications. In general, washed epithelial monolayer with 24 hr biofilms was scraped using tissue culture scraper and transferred to 2 ml eppendorf tube containing 1 ml phosphate buffer. The tube was vortexed thoroughly for 1-2 min to release the cells. Samples were serially diluted and plate count was made on LB plates incubated at 37° C. overnight.

### Applying Phages to Biofilms and Mice Lung Infections

Fifty μl (1-2×10⁹ pfu/ml) of a phage cocktail containing phages φNH-4 and φMR299-2 were applied to wells containing 24 hr old biofilms on CFBE41o-cell monolayer and plates were incubated at 37° C. and 5% CO₂ for 24 h. Biofilm clearing was monitored by measuring light and images taken for times indicated, using the IVIS100 imaging system. Pseudomonas lung infection in 6-8 week-old conventional female BALB/c mice (N=8) was performed according to Riedel et al. (2007). Animals were infected intranasally with 50 μl lux tagged Pseudomonas NH57388A or MR299 in phosphate buffer (2-5×10⁸ cfu/ml). Two hours after infection, a 50 μl phage cocktail suspension (1-2×10⁹ pfu/ml=multiplicity of infection (moi) of 2-5×) was given intranasally. Fifty μl phosphate buffer was given to the control groups. Animals were anesthetized with isoflurane, light was monitored and images were taken using the IVIS 100 system at time points indicated. Animals were kept in an animal colony, and all experiments were approved by the animal ethics committee of University College Cork.

### Biofilm Staining

Pseudomonas biofilms were stained with the fluorescent enhancer calcofluor, (Fluorescent Brightener 28, Cat. No: F3543, Sigma Aldrich, China). In brief, CFBE41o-monolayer containing biofilms were removed from wells using sterile cell-scraper (Sarstedt, Actiengeselischaft & Co., Germany) and mixed with a drop of 0.1% calcocfluor on a microscope slide. Cover-slip was applied; edges sealed with paraffin oil and incubated for 1 hr (37° C.). Stained biofilm preparations were assessed by Olympus BX51 fluorescent-microscope fitted with U-RFL-T fluorescent power supply unit and images taken by DP50 integrated Camera (all from Olympus Optical Co., Japan).

### Results

### Identification of Pseudomonas by PCR and API Test

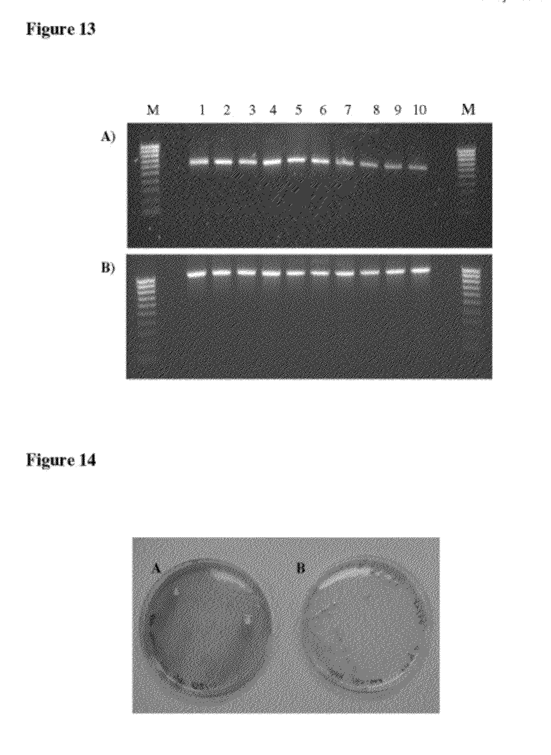

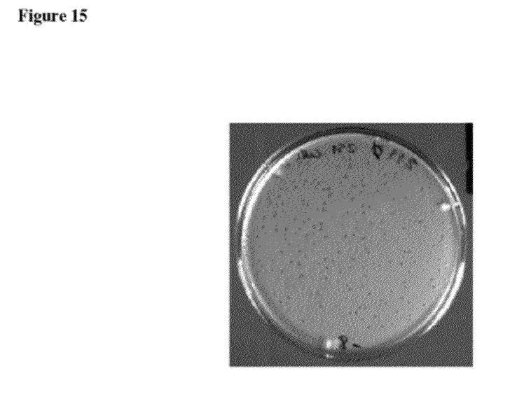

Plasmids and strains used in this study are shown (Table 1 and Table 2). The PCR assay produced DNA products of the predicted sizes. 16S rDNA PCR products of 618 and 956 by were obtained using P. aeroginosa genera and species specific primer pairs respectively (FIG. 13.). PCR results confirm that all isolates belong to P. aeruginosa species. Substrate metabolism and assimilation ability of isolates was examined by API 20 NE kit (Table 2). The API test profile obtained for CF isolates was consistent with the API20 NE identification criteria and confirmed that isolates belong to P. aeruginosa species.

### Isolation of Phage From Sewage

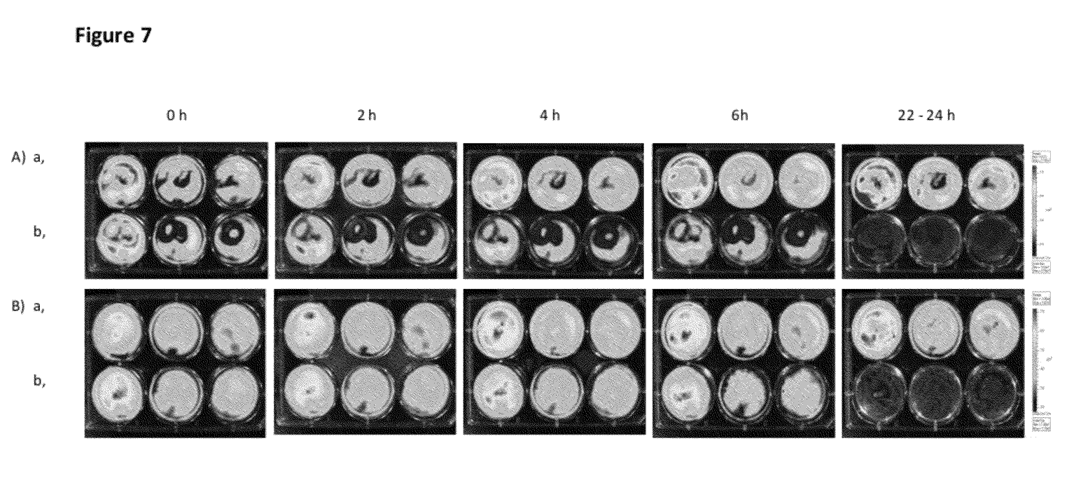

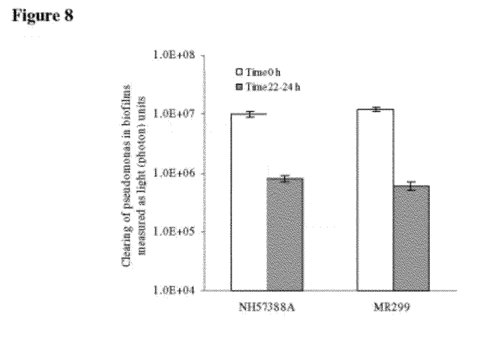

Myophage φNH-4 and podophage φMR299-2, both virulent for Pseudomonas NH57388A and MR299 strains, were isolated from fresh sewage. A plaque assay performed on an LB Double Agar plate for podophage φMR299-2 demonstrated the lytic activity of the M29902 phage against Pseudomonas strain MR299-2 which is illustrated in FIG. 15. The electron micrographs shown, obtained by scanning electron microscopy (FIG. 6), revealed that phage φMR299-2 virions have isometric heads of 40-60 nm in diameter and very short tails measuring 10-20 nm. Morphologically, phage φMR299-2 is very similar to Pseudomonas phage PaP3, a podovirus which has an isometric head and short tail. Therefore, like 4Pap3, the presently isolated φMR299-2 should be attributed to type species coliphage T7 and in the family Podoviridae, as referenced in the Report of the International Committee on Taxonomy of Viruses (Van Regenmortel et al., 2000, Murphy et al., 1995). Phage φNH-4 is different in morphology from that of φMR299-2 and displayed an isometric head of 50-60 nm in diameter and a contractile nonflexible tail (non-contracted tail length=150 nm and contracted tail length=85 nm; tail diameter 20 nm when contracted). The tail sheath shows cross striations and diameter increase 1.5 times when contracted. In addition, a putative DNA injecting structure of 70 nm in length (narrower than the contracted tail sheath) and tail hair fibres are observed (FIG. 6B & 6C). Morphologically, φNH-4 shows similarity to phages PB1, LBL3 and SN, which are classified in the A1 morphological group of the Myoviridae. Based on structural characteristics obtained from microscopic analysis, phage φNH-4 can be classified as a member of the Myoviridae family according to the International Committee on Taxonomy of Viruses (Van Regenmortel et al., 2000, Murphy et al., 1995).

The percent (%) identity of the φNH-4 phage and φMR299-2 phage with known Pseudomonas phages are summarized in Tables 3 and 4. The number of open reading frames (ORF's) for the φNH-4 phage and φMR299-2 phage are 114 and 73, respectively. There are 8 and 3 unique ORF's for the φNH-4 phage and φMR299-2 phage, respectively (Tables 3 and 4).

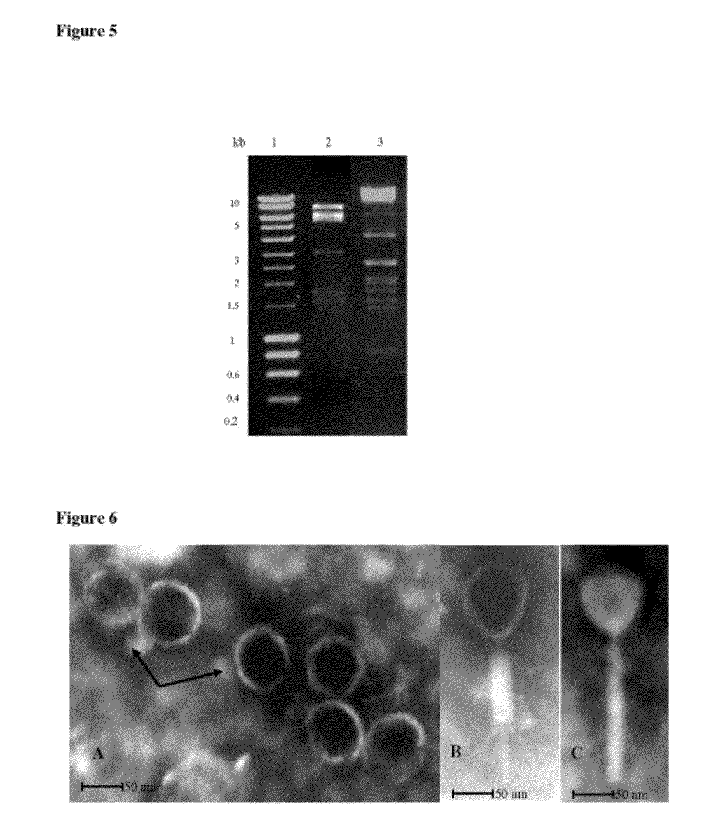

The infectivity of phages φNH-4 and φMR299-2 was tested against 8 other CF Pseudomonas isolates (Table 6) using the plaque assay described above. Six (75%) were sensitive to both phages. However, there was significant variation in the level of sensitivity of the isolates to the two phages. Two (25%) of the CF isolates were sensitive to only one or the other phage (Table 6). This data shows the cross infection capabilities of phages φNH-4, and 4MR299-2. It was observed that Pseudomonas cells exposed to a phage mix of φNH-4 and φMR299-2 produced very strong lytic activity compared to that exposed to individual phages. Based on this observation, a cocktail phage mix was used in all experiments to assess efficacy of phages on Pseudomonas cell lysis. DNA restriction pattern of phages φNH-4 and φMR299-2 are shown (FIG. 5).

Biofilm Growth by Lux Tagged Pseudomonas on CFBE41o-Cell Monolayer

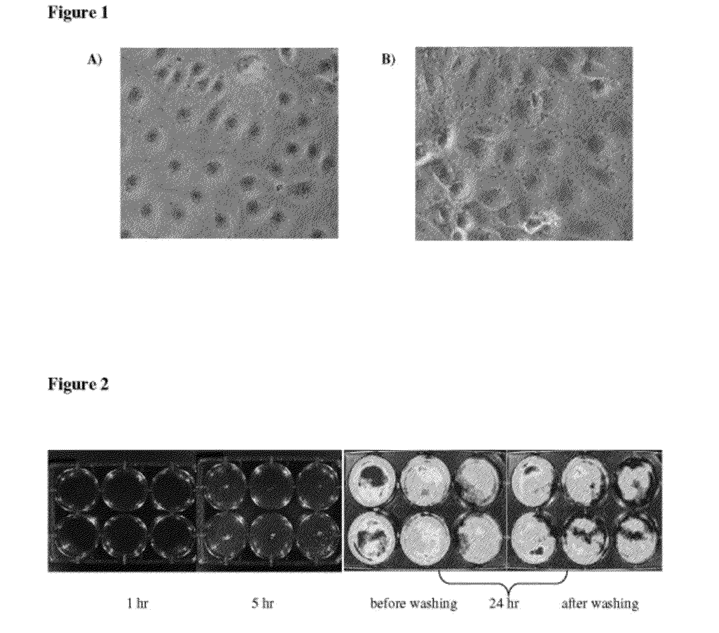

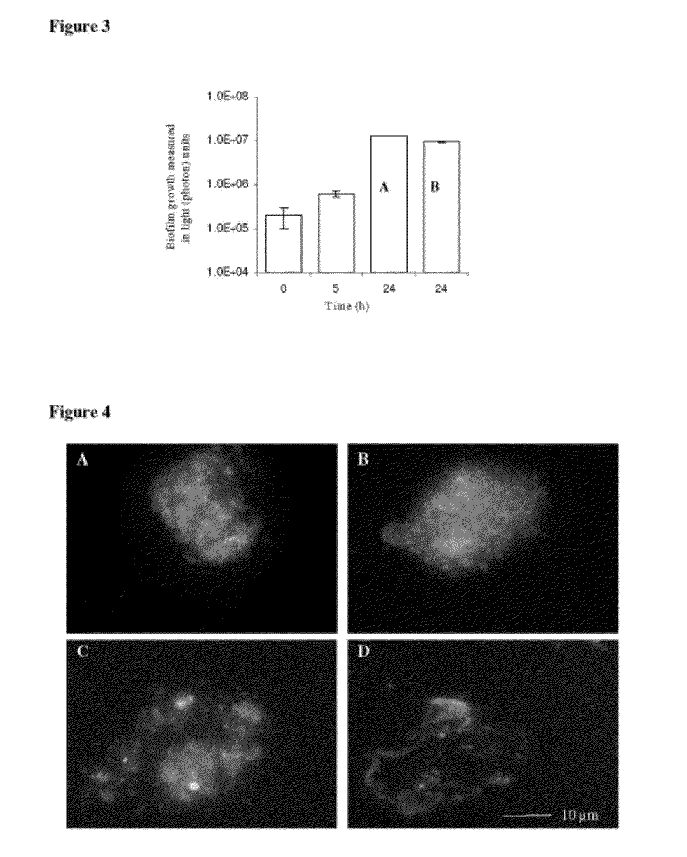

Cystic Fibrosis Bronchial Epithelial (CFBE410-) cells were grown in standard 6-well tissue culture plates. A monolayer culture with a tight junction between cells was achieved in 8-10 days of incubation (FIG. 1A). The monolayer culture was inoculated with lux tagged Pseudomonas strains NH57388A::p16Slux or MR299::p16Slux and biofilm growth was monitored in this static system for 24 hr. The addition of arginine in the minimal growth medium enhanced the formation of biofilms and helped preserve the monolayer integrity of CFBE41o-cells as suggested by Anderson et al., (2008). The amount of light recorded for the growing biofilms increased by 1.5-2.0 log units during the 24 hr incubation period (FIGS. 2 and 3). Washing of the biofilm-monolayer culture twice with MEM medium removed all planktonic Pseudomonas cells. The absence of motile cells and the presence of only adhered clusters of micro-colonies of various sizes scattered across the epithelial cell monolayer was confirmed by phase contrast microscopy (FIG. 1B). The amount of bioluminescence light recorded after washing the 24 hr-old biofilm culture with MEM and removing planktonic cells was only 70-80% of the light recorded before washing (FIG. 3). This confirmed that light was indeed emitted from Pseudomonas biofilm clusters adhered to the epithelial monolayer (FIG. 2). In order to be able to know the cell density in each well, the biofilm cfu count was estimated after washing the biofilm. The cfu estimated for cells attached to the CF epithelial monolayer as biofilms was 2.6-3.8×10⁷ cfu/well and 4.2-5.4×10⁷ cfu/well for NH57388A and MR299 strains respectively. The cfu estimated for biofilms of the mucoid NH57388A strain was less than that estimated for the non-mucoid strain MR299. This might indicate that complete release of cells from biofilms was not achieved for mucoid NH57388A strain during vortexing.

Calcofluor binds to β(1-3) and β(1-4) polysaccharide linkage found in the biofilm matrices produced by exopolysacchride producing organisms. Staining of the biofilm structures with calcofluor (fluorescent dye) revealed the presence of a pack of Pseudomonas cells contained within a polysaccharide matrix (FIGS. 4A and 4B). The biofilm formed by the two Pseudomonas strains NH57388A and MR299 measured on average 20-30×30-40 μm in diameter, and were not different from each other (FIGS. 4A and 4B). The presence of abundant numbers of intact and healthy looking Pseudomonas cells packed in the biofilm matrices confirmed that indeed the microcolony structures formed on the monolayer were Pseudomonas biofilms.

### Clearing of Biofilms by Phage

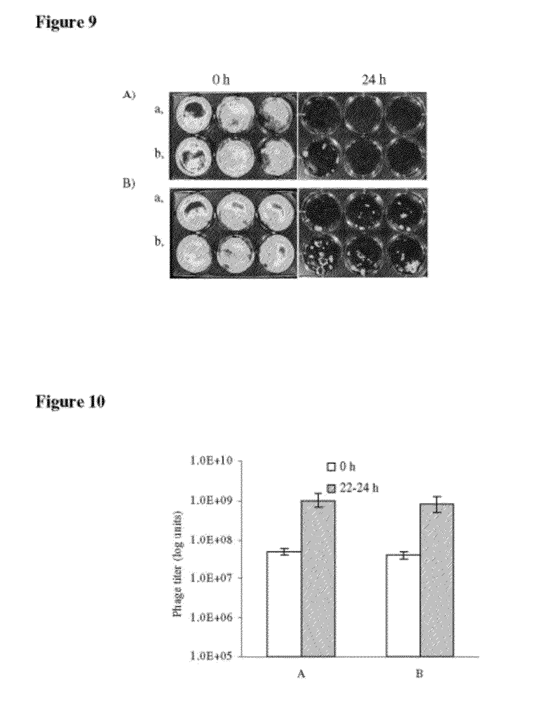

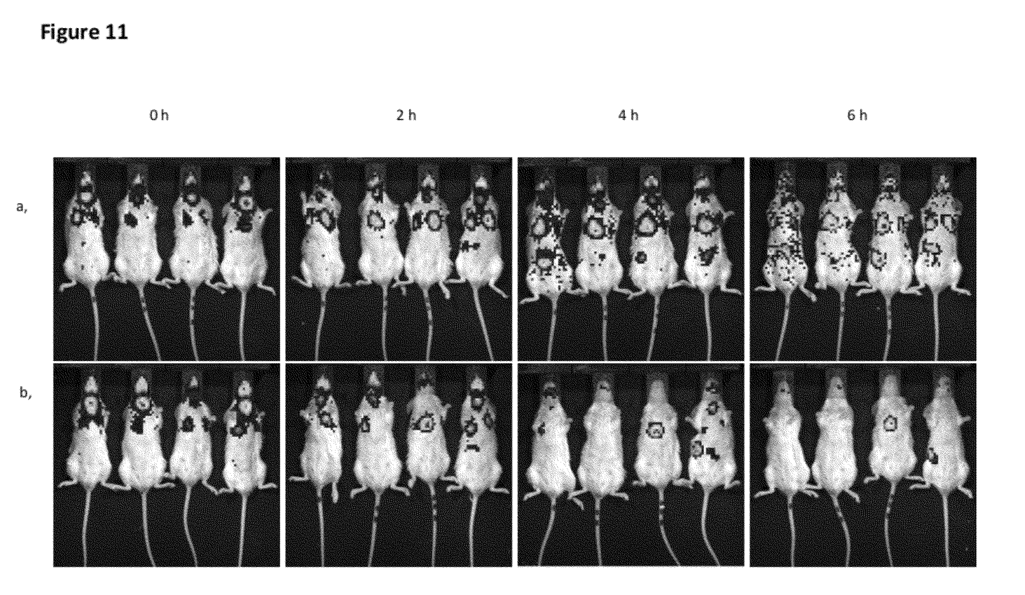

The ability of undefined cocktails of Pseudomonas phages in clearing biofilms of Pseudomonas NH57388A or MR299 was examined. In the presence of phage, the amount of luminescence light recorded for both Pseudomonas biofilms decreased dramatically by 10 to 20 fold over the 24 hr period. Whereas, in the control biofilms (with no phage added), the light level remained high and unchanged over the same period (FIGS. 7A & 7B; 8A & 8B). A similar result was obtained when a defined phage mix (purified from the undefined phage mix) containing φNH-4 and φMR299-2 was applied to biofilms of NH57388A or MR299 strains (FIG. 9). This shows that phages φNH-4 and φMR299-2 were the main virulent phages present in the undefined cocktail. The results confirm that the significant reduction in the amount of light was a direct result of lysis of the Pseudomonas cells by phages. Since the cfu estimated for biofilms was 2.6-3.8×10⁷ cfu/well and 4.2-5.4×10⁷ cfu/ well for NH57388A and MR299 strains respectively, and the amount of phage added was 0.5-1.0×10⁸/well (MOI=2-5) suggests that the extended time period required for clearing of the biofilms by the phage cocktail was directly related to the biofilm structure and not to the phage: cell ratio. Furthermore, the increase in phage titer by 10- to 20-fold during the 24 hr incubation period confirmed that phage multiplication took place in Pseudomonas biofilms (FIG. 10).

Clearing of Pseudomonas from Murine Lungs

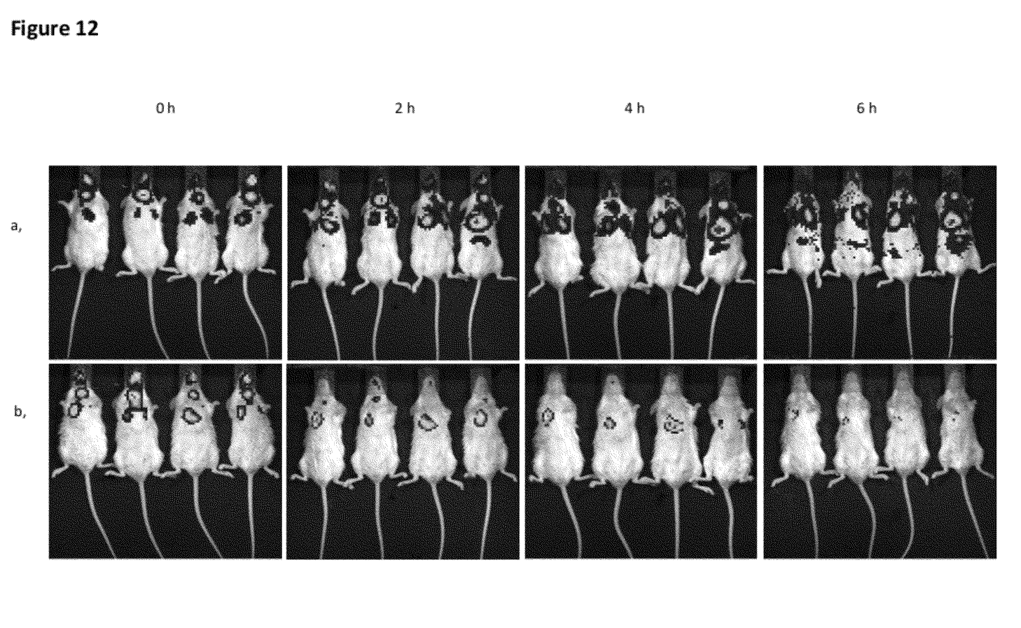

Clearing of Pseudomonas from lungs of infected 8-week-old female BALB/c mice with phage cocktail containing φNH-4 and φMR299-2 phages was examined and results are shown in (FIGS. 11 & 12). Presence of Pseudomonas was evident in lungs of both test and control mice 2 hr after infection. The amount of light recorded in the control mice (without phages) increased 2- to 3-fold and reached maximum level at time 6 hr. Whereas, the amount of light recorded in mice treated with the phage cocktail decreased and reached lowest, and undetectable levels during the same period (FIGS. 11 & 12). The data clearly shows that the reduction in the amount of light over the 6 hr period after administration of phages was a direct result of the disintegration of the Pseudomonas cells by phages. The results confirmed the effectiveness of phages in clearing Pseudomonas from lungs of experimentally infected mice.

The invention is not limited to the embodiments herein before described but may be varied in both construction and detail.

TABLE 1
Strains and plasmids used

| Plasmid/Strain | Description | Source |
|---|---|---|
| Plasmid p16Slux | lux-tagged plasmid vector | 20 |
| P. aeruginosa | Stable mucoid CF mouse | 19 |
| NH57388A | sputum isolate | |
| P. aeruginosa MR299 | Human CF sputum isolate | UCC hospital, |
| | | Cork |
| P. aeruginosa | lux-tagged Stable mucoid CF | This study |
| NH57388A::p16Slux | mouse sputum isolate | |
| P. aeruginosa | lux-tagged human CF | This study |
| MR299::p16Slux | sputum isolate | |

TABLE 2
API20 test results for CF Pseudomonas isolates

| | | | | | | Esculine | | | | | |
|---|---|---|---|---|---|---|---|---|---|---|---|
| Pseudomonas | | Trypto- | D-glucose | | | Ferric | | | D-glucose | | |
| Isolate | NO3 | phane | (Fermentation) | L-Arginine | Urea | citrate | Gelatin | PNPG | (Assimilation) | L-arabinose | D-mannose |
| CH-001 | + | − | − | − | − | − | ± | − | + | − | − |
| MR299 | + | − | − | − | − | − | − | − | + | − | − |
| MR300 | + | − | − | − | − | − | − | − | + | − | − |
| MR325 | + | − | − | − | − | − | − | − | + | − | − |
| MR326 | + | − | − | − | − | − | − | − | + | − | − |
| MR327 | + | − | − | − | − | − | − | − | + | − | − |
| MR330 | + | − | − | − | − | − | − | − | + | − | − |
| MR331 | + | − | − | − | − | − | − | − | + | − | − |
| NH57388A | + | − | − | − | − | − | − | − | + | − | − |
| * Ps. | +96% | −0% | −0% | +80% | −80% | −99% | +92% | −99% | +99% | −99% | −99% |
| aeruginosa | | | | | | | | | | | |

| Pseudomonas | | N-acetyl- | | | Capric | Adipic | Malic | Trisodium | Phenylacetic | Oxidase |
|---|---|---|---|---|---|---|---|---|---|---|
| Isolate | D-manitol | glucosamine | D-maltose | K-gluconate | acid | acid | acid | citrate | acid | test |
| CH-001 | + | + | − | + | + | ± | + | + | − | + |
| MR299 | − | + | − | + | + | + | + | + | − | + |
| MR300 | + | + | − | + | + | + | + | + | − | + |
| MR325 | + | + | − | + | + | + | ± | ± | − | + |
| MR326 | + | + | − | + | + | ± | ± | ± | − | + |
| MR327 | + | + | − | + | + | + | + | ± | − | + |
| MR330 | + | + | − | + | − | + | + | + | − | + |
| MR331 | + | + | − | + | + | + | + | + | − | + |
| NH57388A | + | + | − | + | + | + | + | + | − | + |
| * Ps. | +89% | +84% | −99% | +97% | +98% | +91% | +98% | +99% | −99% | +98% |
| aeruginosa | | | | | | | | | | |

+, Positive reaction; ±, weak positive and −, negative reaction.
*, Reactions of reference strain as shown in identification table in API kit.

TABLE 3
PB1-like Pseudomonas phage genome sequences producing
significant alignments against phage NH-4 complete genome

| | | | | | Genome | | | DNA |
|---|---|---|---|---|---|---|---|---|
| | | | | | size, bp | | Unique | identity |
| Accession | Description | Source | Location | Yr | (% GC) | ORF's | ORF's | to NH-4 |
| FM897211.1 | Pseudomonas phage 14-1, | Sewage | Regensburg, | 2004 | 66 238 | 90 | — | 95% |
| | complete genome | | Germany | | (55.6) | | | |
| FM887021.1 | Pseudomonas phage SN, | SN Lake | Russia | 2004 | 66,390 | 92 | 2 | 89% |
| | complete genome | | | | (55.6) | | | |
| FM201282.1 | Pseudomonas phage LMA2 | River | Maastricht, | 2007 | 66 530 | 95 | 2 | 97% |
| | complete genome | | Holland | | (55.6) | | | |
| FM201281.1 | Pseudomonas phage LBL3 | Pond | Blanes, | 2006 | 64 427 | 88 | 2 | 94% |
| | complete genome | | Spain | | (55.5) | | | |
| EU716414.1 | Pseudomonas phage PB1, | Sewage | Edinburgh, | 1960 | 65,764 | 93 | — | 92% |
| | complete sequence | | Scotland | | (55.5) | | | |
| DQ163917.1 | Bacteriophage F8, complete | unknown | Unknown | 1972 | 66 015 | 93 | 1 | 93% |
| | genome | | | | (55.6) | | | |
| N/A | Bacteriophage NH-4, | Sewage | Cork, | 2008 | 66 049 | 114 | 8* | 100% |
| | complete genome | | Ireland | | (55.5) | | | |

*Unique ORF's of phage NH-4 (ORF; 2, 8, 13, 17, 49, 84, 100, and 107).

TABLE 4
PaP3-like Pseudomonas phage genome sequences producing significant
alignments against phage MR299-2 complete genome

| | | | | | Genome | | | DNA |
|---|---|---|---|---|---|---|---|---|
| | | | | | size, bp | | Unique | identity |
| Accession | Description | Source | Location | Yr | (% GC) | ORF's | ORF's | to MR299-2 |
| AY078382.2 | P. aeruginosa phage PaP3, | Sewage | Chongqing, | 2003 | 45,503 | 71 | — | 97% |
| | complete genome | | China | | (52.2) | | | |
| AM910650.1 | Pseudomonas phage LUZ24, | Sewage | Leuven, | 2007 | 45,625 | 69 | — | 70% |
| | complete genome | | Belgium | | (52.3) | | | |
| N/A | P. aeruginosa phage MR299-2 | Sewage | Cork, | 2008 | 45,441 | 73 | 3* | 100% |
| | complete genome | | Ireland | | (51.9) | | | |

Unique ORF's of phage MR299-2 (ORF; 43, 50 and 61).

TABLE 5
Comparison of Pseudomonas phage PAK-P1 against
newly isolated phages NH-4 and MR299-2

| Phage | PAK-P1 | NH-4 | MR299-2 |
|---|---|---|---|
| Family | Myoviridae | Myoviridae | Podoviridae |
| Genome size (kb) | 93,398 | 66,049 | 45,441 |
| GC content | 49.5% | 55.5% | 51.9% |

TABLE 6
Sensitivity of CF isolate Pseudomonas strains to phages NH-4 and MR299-2. Sensitivity to phage was
determined using spot plaque assay by applying 10 ul phage suspension to a lawn of test organism.

| | Pseudomonas strain | | | | | | | | | |
|---|---|---|---|---|---|---|---|---|---|---|
| Phage | CH001 | MR299 | MR300 | MR325 | MR326 | MR327 | MR330 | MR331 | * NH | POA1 |
| φNH-4 | + | + | + | − | + | + | + | + | + | + |
| φMR299-2 | + | + | − | + | + | + | + | + | + | + |

+, lyses and −, no lyses.
* NH57388A

### REFERENCES

Alemayehu, D., Ross, R. P., O'Sullivan, O., Coffey, A., Stanton, C., Fitzgerald, G. F., McAuliffe, O. (2009). Genome of a virulent bacteriophage Lb338-1 that lyses the probiotic Lactobacillus paracasei cheese strain. Gene 448(1):29-39.
Anderson, G. G., Moreau-Marquis, S., Stanton B. A., and O'Toole, G. A. (2008). In vitro analysis of tobramycin-treated P. aeruginosa biofilms on cystic fibrosis-derived airway epithelial cells. Infec. Immun. 76(4):1423-33.
Bruscia E, Sangiuolo F, Sinibaldi P, Goncz K K, Novelli G, Gruenert D C. (2002). Isolation of CF cell lines corrected at DeltaF508-CFTR locus by SFHR-mediated targeting. Gene Ther. 9(11):683-5.
Capra, M. L., Del, L. Quiberoni A., Ackermann, H. W., Moineau, S. and Reinheimer, J. A. 2006. Characterization of a new virulent phage (MLC-A) of Lactobacillus paracasei. J Dairy Sci. 89(7): 2414-23.
Cozens, A. L., Yezzi, M. J., Kunzelmann, K., Ohrui, T, Chin, L., Eng, K., Finkbeiner, W. E., Widdicombe, J. H., Gruenert, D. C. (1994). CFTR expression and chloride secretion in polarized immortal human bronchial epithelial cells. Am J. Respir. Cell Mol Biol. 10(1):38-47.
Moineau S, Pandian S., and Klaenhammer T. R. 1994. Evolution of a Lytic Bacteriophage via DNA Acquisition from the Lactococcus lactis Chromosome. Appl Environ Microbiol. 60(6):1832-
Murphy, F. A., Fauquet, C. M., Bishop, D. H. L., Ghabrial, S. A., Jarvis, A. W., Martelli, G. P., Mayo, M. A. and Summers, M. D. Virus Taxonomy: Sixth Report of The International Committee on Taxonomy of Viruses. 1995. Springer-Verlag ed. Wies, N.Y.
O' Sullivan, D., Ross, R. P., Twomey, D. P., Fitzgerald, G. F., Hill, C., Coffey, A. (2001). Naturally occurring lactococcal plasmid pAH90 links bacteriophage resistance and mobility functions to a food-grade selectable marker. Appl Environ Microbiol. 2001:67(2):929-37.
Riedel, C. U., Casey, P. G., Mulcahy, H., O'Gara, F., Cormac G. M. Gahan, C. G. M and Hill, C. (2007). Construction of p16Slux, a novel vector for improved bioluminescent labeling of Gram-negative bacteria. Appl. Environ. Microbiol. 73(21): 7092-7095.
Shen, H., Han,F., Yuzi Lin, Y. and Yu. W. (2006). A high efficient electroporation of Pseudomonas sp. QDA pretreated with alginate lyase. Enzy. Microbial. Technol. 39(4):677-682.
Spilker, T., Coenye, T., Vandamme, P., LiPuma, J. J. (2004). PCR-based assay for differentiation of P. aeruginosa from other Pseudomonas species recovered from cystic fibrosis patients. J. Clin. Microbiol. 42(5):2074-2079.
Tenover, F. C., Arbeit, R. D., Goering, R. V., Mickelsen, P. A., Murray, B. E., Persing, D. H., Swaminathan, B. (1995) Interpreting Chromosomal DNA Restriction Patterns Produced By Pulsed-Field Gel Electrophoresis: Criteria for Bacterial Strain Typing J. Clin. Microbiol. 33: 2233-2230.
Van Regenmortel, M. H. V., Fuaquet, C. M., Bishop, D. H. L., Carsten, E., Estes, M. K., Lemon, S., et al. (2000). Virus Taxonomy. Classification and Nomenclature of Viruses. Seventh Report of the International Committee on Taxonomy of Viruses. San Diego, Calif.: Academic press.
Wirtanen, G., Salo, S. Helander, I. M. Mattila-Sandholm T. (2001). Microbiological methods for testing disinfectant efficiency on Pseudomonas biofilm. Coll. Surfaces Biointerfaces 20: 37-50.

1. An isolated phage MR299-2 or NH-4 deposited under NCIMB Deposit Accession Nos. 41729 and 41730, respectively, and variants thereof.

2. An isolated phage MR299-2 as claimed in claim 1 and variants thereof, in which the variants have greater than 97% sequence identity with SEQUENCE ID NO: 5 and have lytic activity towards Pseudomonas isolates MR299 (NCIMB Deposit Accession No. 41731) and NH57388A.

3. An isolated phage MR299-2 of claim 1 and variants thereof, in which the variants have at least 99% sequence identity with SEQUENCE ID NO: 5.

4. An isolated phage MR299-2 of claim 1, or variants thereof, having a genome with at least three unique open reading frames.

5. An isolated phage NH-4 as claimed in claim 1 and variants thereof, in which the variants have greater than 97% sequence identity with SEQUENCE ID NO: 6 and have lytic activity towards Pseudomonas isolates MR299 (NCIMB Deposit Accession No. 41731) and NH57388A.

6. An isolated phage NH-4 as claimed in claim 1 and variants thereof, in which the variants have at least 99% sequence identity with SEQUENCE ID NO: 6.

7. An isolated phage NH-4 as claimed in claim 1, or variants thereof, having a genome with at least eight unique open reading frames.

8. Isolated progeny of the phage of claim 1.

9. A phage cocktail comprising (a) an isolated phage MR299-2 of claim 1, or an isolated variant or progeny thereof, and (b) an isolated phage NH-4 of claim 1, or an isolated variant or progeny thereof.

10. A phage cocktail of claim 9 comprising (a) isolated phage MR299-2 of claim 1, and (b) isolated phage NH-4 of claim 1.

11. A pharmaceutical preparation comprising an isolated phage of claim 1, or an isolated variant or progeny thereof, and a pharmaceutically acceptable carrier.

12. A pharmaceutical preparation comprising a phage cocktail of claim 9 and a pharmaceutically acceptable carrier.

13. A method for the treatment or prevention of P. aeuriginosa-mediated lung infection in a mammal with cystic fibrosis comprising administering an isolated phage of claim 1 or an isolated variant or progeny thereof to the mammal.

14. A method for the treatment or prevention of P. aeuriginosa-mediated lung infection in a mammal with cystic fibrosis comprising administering a phage cocktail of claim 9 to the mammal.


SEQUENCE LISTING (6 sequences)

SEQ ID NO: 1 - DNA, 19 bp, Artificial
  Primer PA-GS-F
gacgggtgagtaatgccta

SEQ ID NO: 2 - DNA, 20 bp, Artificial
  Primer PA-GS-R
cactggtgttccttcctata

SEQ ID NO: 3 - DNA, 19 bp, Artificial
  Primer PA-SS-F
gggggatcttcggacctca

SEQ ID NO: 4 - DNA, 19 bp, Artificial
  Primer PA-SS-R
tccttagagtgcccacccg

SEQ ID NO: 5 - DNA, 44789 bp, Pseudomonas aeruginosa phage
gccggcccctttccgcgttagtatcgctgttatggtctgtcctctggctatcctcgggagtcttcgtgtctactcgtccctaccttcgccagtatctaccaagcttcgccctctgcgctaagccattgatactaacccaacatactccccgtcgcgcttgaaccacggggtctgtcaattgattgcgctgggacaggctaccagtcgggccttctatggtacaaggccaggacctacgctctttcggagtgtgcgttattccctcagctttcgggtttctcccttgttcagtccaaggaccgtcgaaggtacactttccccaggtggccgtcacctgatgggtgcccatgttagaggcttgggcttgccttgtcaaccctctgacgggggttagttcgtccagttctctggcataggctcttttgctgagcggccatcagtcttgcttgtcaaggctacgctaatcccgagggagtcatgcacgttatgccgttgactacggggcgaactataccgcttcaccgatcccttgtcaacccccgatgcctgccggtcttatacttgggtgacttgggcgctcttccctggttggcagcccccggatagcactgcggcaagggggatggtggcctaggtagaacgtgggggccattagacccggttccgcggggtttgtcaagcccctggggtttcccccggtgcctatcggtttccctcaaggcggcgccattacgccatattctttacggggttgtctactggtttttcatacagtctatggactatcagtcccccagcaaacccccggatatagacagtctttgattaacatggggggatcaatggaagtcaataggggatcaagggatattccccccgatccaccccctgaataccctaagatttttcttatgaatacccttgggtaaatccgatgaacggtatctcaaggggttgacccccggactagtcttatggtatcctggggagccctaggggctagtacccctgggagagggccaccccaacgttagcctgggggactctggggggatattccacagatacaccggaggataaaatagacttttggggtactctaggtgctataccccggatcaaaggcaccccgtagactataccttgtggtatattgctgggataagaagaactagacccagggtatagcgacaagaggggtagtcttagggtatacctatagaacccggatcaatagaccttataaccccattaccagtaacataagaagggtagtcttagggtatacctatagaaccctatccataccgttcgtcgggcaatcagggggaattcttaagaaatatcaaaaagtacttgacagggtattcaatttgtggtataatagatctataggcacttaagatttatcttaatttacttattacttattaaatacttaatctatagattatcttaagatagtcttaagaaccttaagaaattccactttccagcaaaaaataataaaaaagatgaaattaggggttgacaaaaacccaaaagtgtgatataatagtattataaggttgagaaattaaccttaagggtgcttaagatttccttgggatgtagctcagttggtagagcagatagctgttaactatccggtcgtaggttcgagccctaccatcccagccattatggcccattaactcagcggtaagagtgctcccctgtctagggagaagtctggggttcgaatccccaatgggtcgccaacaactcctcgtagctcagtctggttagagtgcctgccttggaagcaggaggtcgtaggttcgaatcctaccggggtgacattagagagataggagcgtgcgtatgtcagacgaaaaggttgtctcgattggtgccgctcctctttctgccaaagaaaaactagacctttattgcgaaggactagccgatggattaaacaaaacccaagcctatgtagccgctgggttctcccccaatcatgctcaacgcaacgttgcagcttaccatcggaaacatgccgagtacatcaatgcctttatctctgagcgcatcgggagtcatgtccccatggcgctccgtgtgatcgtctctattgcagaagatccaaatgagaaaggtggtatccgcctcaaggcagcccaggatatcctggaccgaggtggcttcggtgctaagcagaaagtggagcttaccactaagaatgtggaagatatgtccgcagaggatctgatgaatgaagttcgacgaatactcgacgaagagccagacttggctaaagtcctttctttcccgaccgcttgacttcctgagtcttattaagaagcgagaggggttagtactccagtggtacaaagactccttgggatattggaccggaggctacggccacctacagcgtcccggagaggatggacccatcaccctagcccgagcagagacttggctagagaatgacagtcaggcagcctacgatgcagcccaacggcaagtgtctgagctgcctttttgcactccagaactattcgatgccctagtgagcgttaacttccagcttggtacagcatggactaagaagttccccaagacgtggagccttctgaaagccggggagttcgacagagcagcttgggaagccgaggatagtgcctgggctaaacaaactccagttcgcgtaagggacctccaacgagccctttggcgagcatctacgataggtagcacggtgacttaatggatacccaagagcggttgcgtaacctagtcagagaattggctgagcggcagaagtacttccgcatgaaccagtacaccccctatgggtggcaagagaagttcattgcagcctcttctaattgtgctcagctactggctatgactggcaaccgctgtggtaagacatacaccggggccttcatcatggcctgtcatcttaccggtcgttaccctgagtggtggactggaagaaagtttgacaagccagtgaactgctgggccgcagggatctccacagatactactcgggacattctccagtctgaactattaggtgattggaagaaccccgaggcatttggcactgggatgatccccaaagaggatatcgtaaagactgaacgtagggaaggcaaacctggatgcgtccaagctgtaatggtaaggcacgcatatgggggtttgtcctcgctaatcttcaagtcctacgaaatgtcccaagacaaattcatgggtactgctattgacgtcatctggcttgacgaggagtgccccaaggatatttatacccagtgtgtaacccgaacggctactactgggggtattgtctatctaacctttaccccagagcatggtctcacggaaatcgtgaaggacttcctccaggatcttaaacctggtcagttcctgatccatgcaagctgggaagatgccccacacctcagtccagaagttaaagagcagctactctcggtatactctccagcagaacgcaggatgagggctgagggtattcctatgctcggctctggtgtagtcttccctattctggaagagaagttcgtatgtgagccttttgatatcccggatcacttccataggatcattggtatcgacctagggtttgaccaccctaacgctatcgcctgtgtagcctgggatgcagagaaggacaagtattacctgtatgatgaacgaagtgaatctggcgaaaccctcggcatgcacgctgatgctatctacctcaagggtggtcaccagatccctgttgtagtcccccacgatgcgtttaagcatgatggagctacctcaggtcgtcgcttcgtagacctcctgaaggatgaccacaacctcaatgttgtttatgaacccttcagcaaccccccaggtcccgatggtaaacacgggggtaactctgtagagtttggtgtcaactggatgcttactcgtatggagaatggggatctcaaggtatttaatacttgtactaacttcctcaaagaaatgaagatgtaccaccgtaaagatggtaagattgtggatcgtaatgatgacatgatctcagcaactcggtatgcccttctgatggcttcccgtcacgctcgtcccggtgctgtacgaaacagtggatactacaggagtgataccgcaaggttgatccctgattggtttgggagtattgtctaaatggctaagcgtcgtaagatcaagcctatggatgatgaacaggtacttcgtcatctagaccaacttgtaaacgacgcccttgatttcaactcgtcggaactatctaagcaacgctctgaggccctgaagtattacttcggtgagcccttcggtaatgagcgtccgggtaagtccgcgattgtatctagggacgtccaagagaccgtagactggattatgccttctcttatgaaggtgttcacgtctggtggtcaagtagttaaatatgagcctgatactgccgaggatgttgagcaggcagagcaagagactgagtatgtgaactacctcttcatgcgtaagaatgaggggttcaaggtaatgttcgactggttccaagacaccctcatgatgaagactggggttgttaaggtctacgtggaagaggttctaaagcccaccttcgaacgattctcaggcctctctgaggatatggtagcggacattctgtctgacccagatacctctatactggcccaaagtgtagatgatgatggcacctacactattaagattcgtaaggataagaagaaaagggaaatcaaagtcctgtgtgttaagccagagaacttcctggtggatagactagcgacctgtatcgatgatgcccgattcctctgtcaccgtgagaagtacaccgtaagtgatctccgactcttgggggttcccgaggatgttatcgaagaacttccatatgatgagtatgagttctctgatagccaacctgagcgcctagtccgggataattttgatatgacgggtcaactccagtataactctggggatgacgctgaggctaaccgtgaggtatgggcctctgagtgctacactctacttgacgtagatggtgatggtatctctgaactccgtcgtatcctctatgtaggtgattacattatcagtaacgagccttgggattgccgtcccttcgctgacctgaatgcctaccgtattgcccataagttccacgggatgagtgtctacgataagatccgtgacattcaagagatccgttcggtactcatgcggaacatcatggataacatctaccgtaccaaccaaggtcgttctgtagtcttggatggtcaggttaacttggaagacttgctgaccaatgaggcggctggtatcgtccgagttaaatccatgaatagcatcacccctctggagactcctcagttgtccggggaagtctatgggatgcttgaccgccttgaggcagaccggggtaagcgtactggtattactgatcgtactcgcggactagatcagaacaccctgcactctaaccaagcggctatgagtgttaaccagttgatgactgcggctgagcaacagattgacctgattgcccggatgtttgcagagactggtgttaagcgtttgttccaacttctgcatgaccatgccattaagtatcagaatcaggaagaggtcttccagctacgaggcaagtgggtttccgttaaccctgccaactggcgagagagatccgatctgacggttaccgttggtattggcaacatgaacaaagaccagcagatgctccacctgatgcgtatctgggaaatggcccaggctgtagtcgggggtggtggtctcggggtccttgtctctgagcagaacctttacaacatccttaaggaagtcacagagaatgctgggtacaaagacccggatcgcttctggactaaccctaactctcccgaggccctacaagccaaggcaattcgagagcagaaggaagcccaacctaaacctgaagatatcaaagcccaagctgatgcccaacgtgcccaatcggacgccttggctaagcaagcggaggcccagatgaagcaggtagaggctcagatccgactggctgagatcgaactgaagaagcaagaagctgtccttcagcagagagaaatggcccttaaggaagcggagctacaacttgagcgtgatcgtttcacttgggaacgggctcgtaacgaagcggagtatcatcttgaggctactcaggctcgggctgcttatatcggagacggtaaagtacctgagactaagaagcctgctaaggcagtgaggaaataaccttgggagaacgttttggctatagtctagtgctgactgatgcagcacagcagatggtacgtgagaacgttcttcccgagcttttccgtattgtccaagaggagattgagggcgaatggaaggctacggcccccaaagatagtgagactcgggaggcgatataccacgagctccacgccctcaaccgcgtacaacttcgtatccaggcaatcttggattcgatcaccctaaaggagtataagtaatggaccttgagaatcaaggcatggatgaattcgaggcagcggaagcctttggtgagttgctcggtgatgacctcctggcccccggtgatgaaaccgtagatggtaaagtcgtaggtgaggaagaagaggaagaggttgaaaaggaaacccccgagggtgaatctgaggatgagcctgaggaagagtctgaggaagatcctgaaggtgaatccgaagatgaacctgaggttgatgctgaggatgacctctcagagaaactctttgagattccgatcggggatgatatctatgaggtcaacttcgaagaactcaagagtggttaccttcgtaatgaagagtttgtaacccgtcaggctgagttagaggaagagtatgcagctaagcttgagtcagttgatgctgaacgtgctaatctactggccgagcttgagcagtacactgttaccgctattgctggtgctaatcagtatgatagcataaactgggagcaacttaaggctcaagacccggagaagtatcagaagcttcgcctagaggctcttgaggctcgggatcaagcccaggcacttattaaacgtcgtaacgatatcaaggctatgcaggagaagcgagccgagataatccacagctcctacatcaaacgtcagacagaattggctaagaagctgatccctgaaatgactactgatgagacctggggagataagatcgtagcttacggcaaatcaattggctactccgaggatgaaatccggggtattgctgatgcccgccaactggctgtacttgatgcagcacgtaagtgggctgagtctcaggtccgtcgcaaggctgccctagagaagaaggaggagactgaactacctgctgctgttaagcctgtagctcgtagggcggaagcctcagagggttctaagcgggttaaagctgcccgtgctaatctgaggaaagaccagtcggttgaagccgctgctgctctcttctcctctctcgacattttctaatagaaggaagtacattatatggcaactccaactaatgccgtctccactgtagagatcaacggtaagcgcgaagaccttatcgatatcatctacaacatcgctccctacgacaccccgtttatgtcggctatcggcaaaggtgtagctactgctatcacccacgaatggcagactgacgagctccgtcagccgggtaagaacacccgagtcgaaggtgaggatgcaaccatcaaggctggtagcttcaccaccatgctgaacaactactgccaaatctctgacgagaccctgcaagtcaccggtactgcggatcgcgttaagaaggctggccgtaagaacgaactggcttaccaactggctaagaagtccaaagaactgaagctggacatggaatacgccctggtcggtgcccctcaggctaaggtccagcgtaacaccactactccgggtcagatggctaacatcttcgcctactacaagaccaacggctccctgggtgccaacggtgttgctcctgtaggtgacggttcgaacactggtaccgctggggatctgcgactgctgaccgaagatatgctcctgaacgcctcggagtcgatctggcgtaacggtggtcaggctaactcgatccagacttcgagctcgatcaagaaagccatcagcaagaacatgaagggtcgtgctactgagatcaccctggacgcttcggataaccgcatcgcgcagaccgtagacgtctacgagagtgacttcggtaagtacaccatccgtgctaaccgttggttccacgagaacaccctgttcgtcttcgatccgaagatgcacagcctctgctacctgcgtccgttcttccagcatgagctggctaagaccggtgacagcgagaagcgccaactcctggttgaatacaccttccgtgtaaacaacgagaagtccggtgctctgatccgcgacgtcgtagcccagttgtaatctagaggggcccttcggggccccaagttttcctataggagatcatcatgagtatccaatcgacttatgcaattgacgaagaccttgggctctttatccgaaaggatactcaagatgttgagtcaatcctgtccgctaacgccgaagagcgtcattcgggtgtaaacactaaccgtaaggataaccttcgaaaggttgctagtatcccgctggttgttgtagaggcactacggaatcgtccaatgtctgagggtggccccatcgatcttaacctgattggttgcgaccctgaccatgccgctcgatttacccgttggcttaacgaccgagacaactatcgtatgcggacctctgaggcacgggtataagaatggctacgattaataacgtaactgatctcgctattgccgcaatccagtggtctgaccgtcaggaccttacccaagaacttctgatgttgttcattggcaacactactgaccgtctcaaccgtctcctgcgggtacgtgagaatgaacactttgaaaccctgatggcctttggtggaggcatcgagatccctgagcactttgttgcccttcgatctatcacaggggatgccttgatcggtggacgtaccctccagtacatcacccaggatatcttctcccactatgttaactacaactaccagccccagggtgtaacttactacactcgattggggaatttctggcgagtataccctgttgttcctgacggtgccccgttcattgtgaactactggactgttctcccagagctgagcttggctaacccaacaacttgggccctgactaagtacccgcagatttacctctatggtgtccttgagcagatatatctgtacaccatggatgaagagcggtcgatgttctggggacagaaactagaacgggcagtcatggaactccagaatgaagagaatgccgctgacttcgctagctctcgactggcgatcaaggatatcgaacgttaaggagcatccaatgcctatctttctcaaaaccctcgcaatcaatctcctggcaaccctcttcccaaccaaggtaatcgctaagtccatcgtgactggtgctaaagccctggctgagaagactaactccaaagttgatgacgagtttgttaaagtcctggaggacaatctaaaggagaagtcggaggatgcaacttctaactgataacctgggcagcatcgtcagtgccctggtcgtatcctcggtagtgacggtaggttcctcggttgtcggtcagaatgttcatcaggctctgcttgaaagaaacatccaggccacggagaagctcacccaagcggtgagcgatctccagatttctatggcaatctttggggagcggtatgtcacccgaaaagaaatgaaagaagagataaaggaggccaagaatgggtcttgaggtcgcaacttatattaaccagttggtgcctacgaaccctaccggctctgatctgaagtcctttggggatgaccatctgcgactcattaagagtgctattaagaataccttccccaatatttctcaggctgtcactgtaacggcagcccagctaaacgcagtcgctgatactacccagtatgttaaacctgggatggttatcatgtgggctgggtctctagctcagatcccagcgggttggaagctctgtaatggcgtgggtaccacctctaatggtatccctgttcctaacctcattggggcattcccctggggtatcgatggtacttcccaggctgtagggactagaggtggtagcgctaacatcgtatgggatgggttcactgaagggactgcccttaccctagcccaaatcccatcgcacacccacacttggagatcccgaggggctactaccctcaccggttctgcgggtgactctggtgctctgacgggtggctctggtaacgcagccaataccaaccttgagactggccctgctggtcaaggtcagactcacaaccatgcagtaaagatcaacatgccgttgggtaatatcccaccgttctgttctgtgttcttcatcattaagaactgaggtatacaatggccctagagagacaagaggtcaagaaccctacggggattgtgaccgatatcgcccccgctgacctgccactggagaaatggtccttcgggaacaacgtccggtttaagaacggcaaggctcagaagactctgggccatacgcctatctttgatacagcccaggccccaatcttggatatgtttcctttcatccggaataacatcccctattggctcctgtgttctgaacaacggttgtatctagccgacgggactacaatcatcgatgtatccccaggaccttactctgctagtataactaacaggtggtctgtaggatcgttcaacggggtaatctttgctaacgatggggttaaccctcctcatcatcttccaccgtcagaatcaacctttagggtactccctaacttcccagcgaatactacttttaagaggcttaagtcttttaagaacttcctggttggcttaaacgctacaagtaactctgtagaaatgccacagatggtctggtggagtacctcggctgatgctggtggtgtacccgcttcttgggacccaacggaccctactaaggatgctggtcagaacaccttggctgataccaatggtgctatcgtggatggtgtgaagcttcgtgactccttcatcatctacaaggaagactctgtatactccatgcggtatatcggtggattgttcatcttccagttccagcagttgtttaacgacgtaggtatcctcgggcccaactgtgcagtagagttcgatggtaaccactttgtagtaggtcacggtgatgtatatgtccacaacggtgtgcagaagcagtctgttattgatgcccaggtccgtaagttcttcttctcggatattaaccctgacaactatcaacggacattcgtacttgcagaccacgtgaatactgaaatgtgggtatgctactcttccactaggtctgagccaggtaagcactgcgacagggctatcatctggaactggaaggaaaatacttggagtatccgagaccttcccaacgttctcagtggagcctacgggatcatcgatcctaaggtgtctaacctctgggatgacgaccccaacccctgggataccgatacttcggtctggggtgaaggttcttataacccaacaaagtccagtatgatcttctcgtctttccaggataagaagttgttcctcttcggtaataactctacattctctgggcagaacttcgtcagtacccttgagagatccgatatctatctcggggatgatcgaatgatgaagacagtgagtgccatcattccacacattactggtaacggtacttgtaacatctgggtgggtaacgctcaggtacaaggttctggtattcgatggaaaggcccttacccataccggattggtcaggactataagattgacactaaacatgtgggccggtatatagccttgaagttcgacttctcttcggagggtgattggtacttcaatggatacactattgaaatggccccgaaggcaggtatgcgatgagtcaaaagtacagcccttcgattccaccacaggaagaggaggaactgctgcccttccttaatgaagaattcgttagggttggacagactcttaatgacctagctgacggctactggggggtctctatggagccccctaagaagctcaagccagggactgtaaagtattttgcccccggagtggtaggtccagtatccggcatctatcattacgacttggataaccaatggagactcgcaggtactaaacccaaggacctgccgggggattttatcctatttaccccacagaacaaccatcagccgatgggtacctgtgcctataggatgaacactgctaaggatgaagtgtggataaccatgctcatgtctggtggtaactacactaatggagctactgtcctggatcttccccaggcgtattggcctcctgctgagttgtttatcccggcttactccagcatcatcccagcgcagagcactattacttatccacctccgtctgaccctaacgcacctccgttggatcaagtctttgatgttctcaacagggctactattcagactggtgtggttaaccaagcaatgtttaagatcacagctaatggccgtgttctaatccaaggaattccccaaggtgctatcttcgggggtacgtttacttatcccctagtggtgactccatgattcatttgattactcgtgagaatattgactccctcccgaccgtagttccagccctagcccgtgcttttaacaggactgacttgggtaagttctgggactttgagcacttggtgcattccctggttaattatgaggcctatgtcttctaccaggaagagagtgggtatgctggggtgttgcaagtatcccaagcccctttgggcaacatcctacacttcttctggagtggtaagatgcctgggaatgaaactccgatagactaccccgaggttgatgagtacctgggacagtttgcccagaagatgggctgtcggtttatccaatgtgaaggtcgtcggggctggaagcctacccttgaaaagcttgggtacactgaggactccgtaaccttctatagagaggtaactccagatgaacttcctccaatttaacgttaagcggctgatggggttcaacgtagaggatgcccttgatagtcgggcttataaggggggtagtaagaagcaaaccactacctccacctctaccccatatcagcagggcaattacaatgaactactctcgggtgccagttcttggctacataatgggggctttgacccgaactacggtggtgatccgaactttgatccagtggctgaccaaaatgctctccaattgggtggcatcgaggggttgggtaatctcgggggagccctccagtctctcctgggtagctcaggtgttagctctctggctgattaccttggtccttacgatcccaataaaactggcttggcgaacgccattggtgcagccaacgagcagatgcaatgggacttcgacactacggtacgcccagacctgcgtgcaggagcgaccaatgctggccagtatggcagttcccgagcgggtgtagccgaaggtattgctaccgctaggctgctccagaatcaacagaataacgcctctcaactggccttccaagatcagcaggcttacaaccagaaccgtctgaataccttgggtaacctttcggctatcgctaaaggtctgggctctggtaatgccatgcaggttgacgctggttctatgctacagaatcaggagcaacaggagatcaatggtgctctccagaagtgggcctatgagaacaacgttagccttaacgaccttctcgcctataaggaactgatttcgggtgacatgggtggtactaacgtatctgtcaccaagggctctgggggcggtggtggtggcctcggttctgccctgggtgctctgggtggtgcctccctgggagcactcttcggaggccccgcaggagcttctgtgggtatgaacgcaggtggtcgagttgggggtctgttgttctaatggcctctttaactcagaagctgttcaccattggacagaacatcggtggtgggcaggagcgggtacagttgagtcgtcaaggctcctaccgccctgtccacctggggtcgggactaagtgaggaatctagtagtggacaatcaaacccttttggcgcaatgggcggagcagcactcgcggctctcctcgggaaaggaagtgactctactccagaaacagtaccaagcctttctgtcgaaggggctaggggagcaagcgagcaaggcgcagcaaatgttgcagcaggtatgggagcgggaatgggcattcttcctagcgcagaagacctcggctttggacaactcccaaagtcaggaatcctaagtaaactctttggaggttaatcctatggccagcatggcttatgaagggtccccaattcgccctagcatccttcgggctgctcagaatgaactggatatggcgcgaatcgcaaggaacaaactgccgctggctgtagagggctcaggtattcctgatcgtgtccgtcgtgctgctcaggcagccctggagaacccagagcgttggtcccgtgcagcccaagaggttgcacctgcggctgaagctactggtcgtggtgccttgggtcgcatcgcaggtatccttggtggtcctgttggtgtaggtgttcaggctgctgtaactcctgctgatatcggtgaccaagaacgtactcgtgctgaggaaatggctcaggctgcccaggcagtagagaatatgggtcctgaggttgcccagagtgctaaccaatgggcccagggtgtaggccagagggcagctcagaaggccacaggaggcccccagggagccggtctgttgtcttatggtgttacccctaaccaaccctctatcgaacctgagatagcccctgaggttgcctctgaggccggtgcagcggtagcagaggaagaagaggctaaccgtcaggtgatccaacagggtgctatcgaaggtctccgtagtggagccgtaagccgccctgaaatggcccaggctgtagtcgaggctgatgcccaacgggaaggtgtagagcttaaacccgaagagcttaaaactcgggttaatgaagagctcacccagatgaggactatggataatgatgacctgtctcgttatgtatcttacgctctcattggtactggtcttctggcttcggccctagacaagactggtaaggctggggacatgttcgctgcctcctatgaacgtcagcttgatcgtaacctccaggctggcatcaaccaacagaagatggctgctgctgctgctgatcgccagatcaaggagaaggatctggaacgcaaggttgctaaggataagtctgatgagcgtctggggattggtaacctagaggttaagcggggcactctggaagaaacctctcggaagaatgagggtcttctgtctaaatgggaaaaacaggctgctgtagaacgggccaagcttgccctaacccagcgtggtcaagacctagctacccaacgggctcagctccaggctgaaaccgctagacgtggacaggatatgtctcaagagaatgctcagcttagttctgcggtacgtctcaagactgccaagatcagtgctgctgcacgtaaggctgctgcttcggcttcccgtggtgaggctatcactaccaaggatgccaatgagttggtcaagggtgtctctggttcggatctggtaggtggtgctaagattggtgaacaagaccagtcggctatctcccagaagcttcggaatcacctgaggcagaatcctggggctgaccccgttaccttcgtacaacaggaagcggctaagctacagaagcaaccgtcgggtctttttggctgggggtcaggtattttgagtcgtcctgcccctaagtgataaaaggtacttgacaaggtattaaaactgtggtataatagatctataggcacttaagattatcttaataacttattgtattattaattaaatctttaggttatcttaagatagtcttaaggtgcttatcttctattccagcaaaaaggtgacttatggctctttcccctgaaatgaaagccgctattgctgcggaactggctgaggtaaaagacctcgatgtagccgctgagttggcttcgttgaatgagccagaagaagtagaggtagctccccaggaagtaaccccggaggttgtacctgaagctgccccagacctgagcgctttggctgctccggctgatcctaattctattggttctgcgatcaatcgtgggatcgataccatgcagtctaacatcggtggcactattgctactgttggtgagctgactggtaatgactacctcaaagattacggcactcagatggctgaggagaatgcccaggaggcgtcacagtacggaaccccagatgttcggtcttttgccgatatccaagatattccctccattggttctttcctaaagaataatatctcagaagccctcccctcaatgggagccgtaatggctggtggtgccgcaggtgctaaggctggatccatgtttggtgcccctggtcgtatgggtggtgccctcataggttctttcctgagctccatggggatcaacgtgggtgctctgaacaaccagatgaaggagcttgaccccgagggtagtaacccctggactgctgtcctaggtggtgctggcctctcggtactagatactgcgggtgctggtgttatcgctgccccactcctcaaacacatcggtaaagatggcgcttaccaaatgttggtccaatcgggcctacctaagcagactgccattgaggctgttactcaggctggtaagcatgcggctgtctctggtgtggctgagggtgttacctctggtgcccaacaggccctacaggacaccattgcttatgatgcagtaggacaagtacaacgtccagaagagttcatggataacctactgacggctgcattcgctggatctgccatgggtgtaggtgggggtgctgtatcctctggtcttaacactatggccagcaacaaggattcagcaggctcagcccataccgatcctaacgccccttcggctgctcctacggcttccgaaatggagcctaggggtactatcaggaaggcctgggatgccatgggtaatgaagctacggccctcctggagccacttgctaaggcctccccgatagcccgtgagttctctgagacctttagggctgacatgagtggtgagagggcttctggtaagaccatcttcgaggatcaggaactccaggctggtaagtggaacactgccctggataacatcatcgaaggcaagtccaataaggagatccgccagatcatcaatgatgcctctatgggcgtcaatactcctgaggctaccgccctccgagatatcttgaatgatgttagggctgaggctagtggtcgaggtggtatgtccgttgggaatatcgtcaactatatgccctttggactgtcccgtaagaaggtagaatcccctgagttcctagatgatattactccttatttccaagaccgtcaggctgctgaggacgctgtagctaattggctggcagagacttcggatgatacccgaggtaatactgccccagaagttacccgcctagtcactcagaatcagactacaggtgcttgggaggtagatccacggtatcgtatccagggagatcctgatacccttcggggacggtttgcccagagtgatgcagtacctaagtatggtcaactggaggaaagtcgttcctttggatctgtacctcaggagatccttaataaatacaccctcaatgaatcccctaaagacctcatccaagagatccgggattactttgagggatcttcccaccggattgcctttaccgagcggtttggtatcaacggggagaaggccaatgctatgattgcttcggcagtagcagaggctcaacaaaatggtaagcgagtaactaaggaagaagtagaccggatgtatgacctagtggatgcctataacggtatgcacggtcgtataaaagatccgaatatcaagaagctggctgctgtagtgtctggtggattggtcttgtctcgtcttcctttggctggtctctctaccctgaccgagttcagtatcccatttgctaaggctggtcccatgactgccctgggtgctgttcttccaacagtgggtgaagttgcccgtcaggctactcgtagtgtattcagcagcattcctaaatctgagactgggagactcatgagtgatatgaaccacacactggcctctgctacctctttgatggcagatcgggttggtgctgaggtattcaaccagtacactcagaaggctgtacgtgggatgttcttggtcaacggtctgtccatcctgacccatgttgaccgagtattcgccacagagacagccaaaagggtctatcagaacaacctcatggacctcgccgctgggcttccctttagctcagctaatggtgcccttaaggttgctcaacttagggaaatgggtgtgaacgtgagtagccaagcggatgccctcagattggtatccccagcaaccccttcggaggttctgatggccaacaacgttaagaccctagctatccgtaggttcgtagaccagacggtccttgacccaactttcgctgacaagcctatgtggatgagtaatggtaacgtccagatgttcggacttctgaagggttatccagcagcttatggtaacattatcctcccgatgatgcgccgtaggttgtcccctcacttcactggttcttggaccaatgctgggatgggggctgcgggtgtagccttcacgcttggtctgatgatgagcctgggttacatccaggatgaactccgtcagatggctaagtttggtggctctagtcgtgaggatacccgatcagaagagcaaaggattatggatgtattgatgcaacagatgccactccaggcctctatgatctacgatatgctaacagcttaccggagaggtaccaccccggcagaggtactcctgggccctgtcgcaggtgcagctaccgaaggagctctggctgctggtaagactattgcatccttcaatgatgatccttccgctggtgagatttggaagttcttgtataagcaaactccggcccgtcctttcgttgcaggtatggaagcaatggaagacgctctggatctataaggagaagtatatggcaacttacgttgaaactgaagcagccggcccaggtggccgagcctacgtcgtggacgtaccccaggtattccgtcgtaacaaggatggggatctcattgagattactgctgacgctggacgagtgaataccaacattgtaactgctacagagttcctcccggactattacgcggttaaagagaacgcagccctggttgtagagaacgtaactgcctcttaccccgaccatccctcggaagcggtagtccccagcgtcctcgtgaacaccctcatcggtgcccctggtgagacttctggtggtaacgtagacccaggtcacgtcatcggtttgttctctgagactatggttgaggccccttgtggtactgccttcggtagtgaattccgagtagaccctcgacgggaacacctgggtgtttacgtggctattaagcacgtgattggcccggatgaccaaaatggtggtaccatcggggattacattatcgagcagtttgatgacatgcgtggtcctgtgaagaacattggttctctccaacagaactacctggatccccgccttgtgactactcacctgggtggtaatgtggtcaacactgcacaactgaacggtaacatcacacttacccgacagaactctggtcagcacttcatgagtatgtcagctacggatatcaccgtgactttgggccccgatgtaaccccaggttgtgtattccactttgtgcaaggggctgctggtaagatcaagttcgctgtctccccagacaaggcttggtatgccaagggcaaccagactgaaaccgatgctcagttcggtgaatgcactgtccgggtataccccttcggtgggactgttggttctttcaagagtggtctctaatggacccaatgtctattcttattcgggaagtgaggtaatctaatggcaacttatattgaagatgcaaatggtgaaagcgtattcgtgattcagtccaaggctattgccgttcggaatgaagacggtactcttcgtcatgtgatcggtcccaaagaccttaaacctgttcaatgggacgaggtggaggggaagcctgagcttctggaggtgggatccaaggctaacgaggctaagcctgggaattggaaacctaaggcttccgaagttgtaggtctggatgcagtagttaaagctgctatcgaagaccgtatcaaggatatccccgtagcatcctatcaggatagccacgaggcccttgtggataagtttaacacccttctgaacgcccttcggggctaacctaaaggccccttgggatttacccttggggcctttttttggttaggttcagacttctacggggtgatacttgactacactctcattcttggtatcagcgaagtcccgaatgaagcacatggcgtcctgtagggtttcgaagattaccgggagggttatgtcactgagggaggggcggaaggggtccttatgttcactctcaaagtttctccaaaggaacgcctttttatactgagggaagaagtactttccaacttgctttacacgatatttcataaacttcccacccatgacaatatctcgaagaatagacttaagagttctcccatcgtcaggagtaacatcagtcaactgtatcccacatagaccagaatagcaaacaggatcgatacgaatatgtatgtccacgggaaaccgcctttaaccttttgatcgttcttcatagcccctccaatctccatagaatacagaggatcgttggtccaccagatccagcatagcctccagcatttccaaccgctctttagcctccagagctgccttcttgaaggtcgctagctttgcctcagacttagccagttgaccctccatttctgtcagaagatcctctagttcagctttcgtcatagtacaccaccttgtcactgcttcttagaacatactcagcgtcaatagtccaactcatggtcgggcatttgatccgaatcatcttgggggtaaacccatcgacggtgccaatggctagatgggaggaggtgttcctgtggaccaggaagactacactgtcccctacctctagcttacgtcccactcgatctttcatagattaccccaagatatccggaaggtgaacgctggggatgtagaggatctccaggacttcgatggaatacatccctcgtctaagcccctcctcagcaccacctttcccaaacttcatatccagccagagttctgcggagatcggcgtcttaaacgccaacccccgttgaccgaagtcatgctcagacttaatgtagtacaggtctggcaagaacttactcacaggaactcctccacactcatcagcttgaggtcatactctccgtccttgtggttccggaggagggctacacctcgccagtggtcgttaccctggggccctttgtaggactcatcatgggtgtagaagctaccgactacaagcccgtggattcgcttcttgcctactgcctcgatgtggtacttgaatccctgttcgtggccttggacgaagctgcgcttgatcttattgaggcgatgctcggcactcccgccgtatggccgaccactgttgggattgtagaagtagtgggcgaaggcgacaccttcgataacacccacgtcgaggaaatcatggacaatccaatcggacagatcaaaatgatcgtacccaataaaaccttggagagcagggttagaattttcataccgcttgatacgctcctcgtggttaccgatgaagaagtgcatttctggtcggtagactctcttcttacaactcgcttggtgacgctgtaggcggcgaagaggattgagcagcagccgcatcgcatcattacccgcttggatatcgctgaggactcgacgtccttcgatcttggcggtaccacggtcgtagcttgacagactgggcatgtcccaatggtccccaatatgcacaattcgcttggggcgattacgtgcgatccattccccgagattcccgaggtggtcaacattgacccctgggcggacttgggtatccgcaatgacgagggtatcttcattatagcgcttactcattatggaaggactccttgaaagtctctagagtgaaccagcggaagccatttcgttcggcccactccgccatagtttgctttgtaccatctttgcgcttcttaagccaatggcacgctgtatcgggcttctcgaagatgaacacaagttctgtatctggggggagactctccctcacccagacatacttggccgcctcagacgcctcttggaagtaccccttgacctcgaccagcaaagtcttcgttgatgagccagtctctaaagaggccttgaagtcggggtaatacttgtgcttcaccacgtaagtgagtggttgcggcttgaactccatcgacggcatcgtctcgtggacgatcttctcggtgttggagtcgtagggcgtcccaatcaatgacttcagcttgggccggaagaatctcccctggacgctcaatgatattctcctcttgaagtctcccgataaccttcaggacactatggctggtattgctaccaaccaaattgacccaggaaataaggttatccgagagtttgactttatggtcagggtatttctccatgaccctacggatgtgattcacaagacgatctactgctccatctgagataatgttctcatgaacagtgtggccaatcaacgggtcagcaatgttaatgagggtaatccacttattgcccttatccctggtgtagtcaggagagtagagcaactgacgatccccattccagaagctgatactgcacaggccaatgtcaaatacgcaggggtgctcctgcttacacacaatcaggttaaccgggaggtgtccctcggggaactcacaagattcccaaaccttaaacgaatcgggatagtcgttattaacctctttatcccctagccgtactttcttatggccaatggcgttgaagaatcggatgacgttaacctcaagggtatcctcatccacttcctgaggaactacacggacccgattacggatggtgacctcttggagggtacggggctctacaacgtagatatcaatatccttcggcacaaccccgttgtcacaatcccggaggaacccgcctccaacccgaaggccagtgaattttgtggtttcctctagggtcttgaagagttcccgacatactctaagggcctcttcaatagaatagttctccatcttcataaggtatctcccaatgtcttattactcggcaattaagctcgttaccggactgttcaatatcaaaccaattacggtctctgatgaggtagaggagacgaaagttctcagtaaggtactccttccaattatctccatagaccttttggtactcgtaggcgcaagcctggaagagatccttctcagtagtaaggtgcccaagcaaatctgccgctgacttaggcccgatttcaggacaacctggtatgttatccacagagtcaccaacaaggagttgtccatagaggaatgccgtaccgtagccctcaagcttccaggacttttggggcttcttcttcccatagagtctccaagaagcggacactcgtccaaggccctcgttacgaaagggtcccaattctggctgcttatcgccacaggcccaactgtaatggtggcactcggtgatttggcggatatccttgtcgcgagaacatgccgtaaagttatccccctcctcagttccccgaaccccgagccaatcatcagcctcgataccgttgacagtgactgctccccacttgtccttaagtcggtctgacactgtttgccagtgatgtggctttgtatttccgacgcgagtccccttatattggcggatcgtcgcaagctccaatcggaaattatcttttccagtgaggtacacttcatagtcatcagcgcctactctctctatgatttgttctatctttttatctacaagctcgttaacttcttcctcgggccagggcctaactacctcaatgtcgaagatgtgttccttcttcatagcgatgtggcctagctcgtatcgaagtacgtcaccatcaatccccgcgatcatccttagagccctccggacctttcttaagaatctcttcccaatgcttggtgagggcaaatgcttcacccttggaccgatactcgggatgttctaccacggggttggctagagcttcgtagcccttccagtcataatactcactctcttgacgcttggctttctccatagcagcatccgtcaggctgtagcctggggtttccccaaggtctacctcaaggcctttgacagagtaattgctgggaatggtcttagccctcggatccagtagaagttccattagaaccctcctcttgttggatttccagctctagggctgctagggcgttccaaacttcgtgataccggtggggtagattactttgggggtccaaaaacttacccttgtgtcgctcaaggaggtgtcgtccgaaggcagcatcatatcgctggattccgttctcgacgaacctccaacccccgcgactatatttgcttgctccgaagttagcaacttgtgccaccgcataaagagctctcgggaacgcttcgaagatgattccaacatccaccttgcccttatcgagcttagcccccggatctcctgccttgattccattgggatccacctctaccttatcattagatgcagctatagaacttaccactcgtacaccccaaattctctcatagtcagctaagcagtaggggcagcccaaagggcatacccccgcctcatgatcctctaggctcttagataggagcatcttcgtcatcgaagtcctcgccaaatacgtcctcaggcttgtcctcttcctcggtgaaaccaccaccgaacagtggatcatcctcaccatcaccaccagatacaatctcgtggagacgtacaccacggagacgaagggcagtgatagtgcctttaccccgagcacccttctgggtttcgatcaggataccgacttcgccagtagagccaccccacggattggggccatcatacggcttgttgtcgctggtcaggacaatcggtttcataacagcggacttacgctcaccatcctgcatccagtcaacgtgcttcttctgcttgaccttgaacaggaattcaccgtcacgctcggtgtacttgatggtgcctttgaggcgtgaatccttggactccatcttggctaccagctcatcggcttgctccttggtcggaatgcagatagtaacttggtaaacctcaccgtactcggtatccggagttacgatgtaggggaagatcagcttcgctaccgggaaggtgaatcgttgggatactttcttagacattaaatatacctcttgtagggaagaagatattatagcacaaaaggggtcttaagtcaacccctagatgtatcagtgtgtgtcataccaggacctacctgcgatagaatcactggcaagaggacagttacaacccaagatttccccagcttccttgacacaagtctccatgaatccacggagagtcgcaaggtcctcagggtggcactcccactggccttcatcgtggatatccaaaaccttccacgccttgagtccagcccgtcgtaccttctcatctaggatcaccattgcccacttcatgacaatagcaccagcagcttggaggagggtgttcaatgccttgtggaccatcacctctccagactcactacggcgcatcgttagcttacgtccgtcgagtcctacaaggtagcccctttcggcttcctgcttaaccctctcaatcagcttagccagctctgggttggccttaaggaattgctccctcagcctagccccatcggccctcgtgccaccaatgatctgcccaatttttgcatccccagcaccgtagatgaaggcgtagatgaacgtcttagcatcgtctcgggtaggaagccctgcggctacttggttggcagtatggacgtcaccatctacaacctctcgggtatagtctgggtcgccaatgtaggaggcaagcatcctaagctctagaccagcaccgtcatagcccacgaagatcagttgattcgctgggacgtatactcggtacttgcctaccgtcttccacttaccacctttgaggacctccacgatgttagtgtggggtcttaccctttcgttctcacctatgtcgcggcggaggacagtccatttccattcaccagcattaggagtcccctggaacaaccctcggatttcaggcccgaagggagaacgagcagcggggatattaactactacacggtgtctcatacggcccgtgggtgttgcacaggggttagcactcccctctaggcgtccattaggcttcacctgctcgactaatccctgaaggagggagcgtcggtgagcccaaaccattcgctgcatcacaagcttaccaagatccccctggacggtatccatggaggtctcagtcatcttcggcgtagtgagaaccttacgttgtttctcaagcttcctcttgacctcaccgactgtagtcttcccagggatgattccctggagcttcattcggagtagtccagagggggagcttcggaggtcttcgatgtagcggttcaaggcaccattcagatcggatccacgtagcttcttgccatcagtgccgactgtaatgtccttgatattccactggtcaggtatccagccttgagacagcatccagtctttgaaccttgcggttttacccaggtcaaagtccttgtactcgattgctgtaaagggtccagatacctcgaagtgacccaatcgttgccatagagcgaccaagcgtttgttaggcagtccactctttaggaatggcttactgaatgctcccgcttggacgatcatgggaggcatctgggggactgcctctaggtcaatcttgaggattcgttcgttgagtacgtggataagccacttagcccgttgagtctggaagtagaccccccgtttactctggcggctaatgatatcagctaccttgtgctcaacgaacagtgactttctccaatccacttaatcccccttaagacgctccgccagattctctactactgtctccacagcttcgtgggatacaccctccaggtaggagaggtagtccacggtaccactacggtccagcttccattgctgctccaggaaggctacggcaacctgctttagaaggtagtcgtcgatacgctccatcagcacacttttcactttactcatgtcatcaccaatcatggacgaaggtggatacgcccctgcaaatagcaaggtccacaaggaagttggccaggtcaaccctacgttgctctttacggatacgctcctcactaccgttgttcttcaggtagtagtggaggttgaaaagggagtcccgtaggaggttccgaggatcgaacgtttcgtgacggtcaatgtagaagtcgtcatagaggctgagatccttgaggacaacccgttgttgcttcttgtcggaataccgctctaccttaccatggccactaaaataagtagagccaaggtagcctttgggcagcttgaagaagatgctacgtgcagcctggaggggtactttgggattcatcagataggctcggatgaatttcttacgattctcctctgatttagcttgggctatggcatcagttaccatcaggttacatgcgtagtgagattccatgtgtctctcctttagatataagagcaacccccggcaccacaaggttattataccacataaagtaacttttgtcaagcctttttgtggaaaaccttgtcgtaccagcgatagaagtattgggcgaaggtaatcccaagggcaccaccccaaccgctcacaaagatgaaccatcctacatccagatggctcgatcctactgccttgatataggcgaactgagcgagggtgatgcaccaggagactacaaaaccagccttgattttgtcatccctgagaagcttagagttcaaccccaagaggaagacctgaaagaatgcactccagaagatgacaaggacttgtagggttactgacacgactcacatacctcgggatctaacccgcaggcattacccagctcgaagtcatcagcatcgacctcgggaccaccccagccaccactgaaggctgcctctagggctgcttccttgcgggcttcatcctggggcgtaggcttgtacttattgtccatcttctttctcctctacggaagtccatccacggactgcgcctacttctaggctgcggttgagcaaggggttagaatcagcctcgccttggaagaatcgaatgatctctccttcgatccgatagaagtcagcatagaacaccttggtgtcctgaaactgaacattgaccacgtacttcttcatcctagatcctcaggggcccgaaggccccacctcatttactttttctttcgaatgcctttggccttacgattcccaacacgaccaccatcagcagcattagaagagtggctacggacacggaggttagaatccccgttaccaccaccatctttaacacgtttgatgtgatcgaccacctcgtcagacctgagcttccgtccaagacgcttttccacctttcggcgggcgcggtgccgttgagcatctcctgacttagatccaacgcccgtttcgccgcgtcgtatagccagttctctttcacgcttgtagttcctcacacgtttagccaagaacctgtcctccactctactagctcctcccaggtcatcccgagaaccttaagcaggtagttgaagagtgccatactaacaactccatcctgtaggcaatactcaaacatttcctcggagaacttggagaagtcgttgaaatcacccttgtggcacttcaggaatcgaccccaagctcccagcgagtgtcccataggacggtcaggccaagtgagtcgggaggcgaccaaggtatcgtaagaccgttcttctggaatcacgataccatgaagcttctcaaggaccctgttgtcgtagtccaggatgttgtgcccgactacgaactttgcatgggcgatgatcctgtagaatgtctccatttcctcaggccggaatccccggactacctgagttggtatatctacaagaacagcgcaccaaagccttgtgacgttatcgtaaagggcatcaccctctaggtcgtagataacgtaatcaaattggggactcgtcgtcctcgggtacttcatcatagctaccttcacgctccgttaggcaacccgtgtcagggttatatttggtgaacactagcccggtgttaccgtagttccgatccttgatgacccggatacggctctcgtgcttctcttctccagaggcttgcttgttcctctcgaagccaatcatcagttggcaccaacgctgcatcgcacggctaccagtgaattgactttccttcacctcagccccttcctcgtgggtacggttaccctttggcgggttaaggtgggagaagatgaatactcggatacccaactcgtcagccatcccggccaactctgtgcagatacgagcaatctcagtgttcatttcggatggagacaagtggttagtcatggcagtcatgttgtcaaggatgacggtcttaacatccatagcgaccgcccaatagcgaatacactccttgatatgatcccaatcattctgacccttgttcttccacatgaagagcttgccccgaagcttgccagcagcatccttgaacgcctctacgtcccactcaagatcgggccggtggaatgccctttgagctaccttacctgcgatgttcttgagtgtcatagcaacttgctcttccagaaggaaggttcctacgttttccccatgttcgatgatattccatgctgcaatgtggtgagccagcaaggtcttaccaatgccagtaccaccacctacaccaacgatttcaccgtcgcgctgacccatagttagattcgtcaggccttcccaggggtaactcttgccccacttaggtggctcaagggcttctgcaaagacctcatctacggtaacagcacagtctggggatttcaccttactgccgaatcgaatgagctgatagacttcatctgcccggttgcgcataagcatgtcgttagcatccttgaggggcatttcgacaaacttacagtcagggaggagggcacggaccctctcgacagacttgcgaccttcctcgtcagcatcgtgacagtagacaacctcttggaacctatcgagaatcccagattccttgaggaaaccaatgtctcgaccagcagacccaactccagaagaccagctaatggatgcctgccgtaggtgtttccacttctctggtaacttactttccatgatctgagcaatcgacatagccgaaagctcgtcctcagagataataagcttaaggtgtcctacgtccttgtgaaagagcgtctcaaggccgaacggatcgaccccacccctggggctaccccgatagtaaaacgccttaggatccaagatccggacgttatacgcacggtatttgccatcctttgtccgtgggtagtagtgttcgatcacatctacaccattctccatggacaatcccacacgaaccccatagcgttctgcgatggttttactgatcttgcgggagtcgagggccttgaaggggtattcttggacctcagcgacttcccggaggatatcctcttcggatttctccgttcgcttaggtactacaaagccctcgtccagaacctcatagtggccacaacggttgcacttaccgaagcggctacctttttcttcgtcgatgaacaagatgaggtgattacctgtgtgatccccacctttagctcggcaacccggacaagccgtatcaccgatgattttcttcatttaaccctccagttgactcttgaggaccagggaaggaaccgggttaacattacccggataggtcagataggggtaggagaagtcacccaattgtttctccagcaggcgcaactcatcttctacggccttgagacgctcccacatgaagcgaataccagcagactcgtcatagtacccgctgtaatccgagtcatccgtagcaggctctacagacgcactgatggagtcctgtagttgtctctgggatgccatcaggccgatgatccgacggaggagctgacgtttcccctcatcatcggttttcatcaggttggggatatcacggtaaatcttgtgggccttaactacatcagtatgggtcatgcagcattccttttattaacaaatttacgccagtcagcaccctcgacgatatgcaggtcttgatcttcgtcttggtggtagacaagggcggtaatcatttccccgttgatacgaaagttcttggggctacgatcgtaccaacccggataaccctcaaggcgatccaggttgtttaacatttccttaccttcgtccccaccggggagttcccacacttcacccgtgacaagatcctcacggtgtttgaaggacaggatggggaatcccccgcaggagtacatacggcctcgctcttgggaggtggcatgcccgacgaatcgggcgccctctagaagacagtggttgccgaagccgtgcatcagtgttccataaacaaaaacaaacatcagattacgccctcttcgtagattttggtgaatgcctgggtgtatgcctccagggcgctacccccttcatccagacccggagcagtgttgatctccagaacgtaggcgcggtcagtctcgtgcttgtagacgatatctacagcaccgaagtccaggccacagaagttgaccgcttggattgctgcgccaactgcctgagcatggccctggcgttcttcggcatcattgacaccgtagacccagccgccggctacgttgcggacgatgctctgggaatcatctgccacattctcctcgacgttacggcggagcttaacctgggacaggatagcccggccacgcaccacgtggattcggaactcggtacgacggcctacgatgccctgggtgaacagacgagcattgattacttcatcggggatattcgtaacacccatgacatataccgggtactgcctgctgcggatcaactgctggatagcctggatttctgcatcccgagtagacaccagcagggagatcccctggccactatggccattgagttgggtacgaacgaagatacggccaccaccattattaaccaaggcaacggcgtcatcgaagttgttagtccagcgagggatcaggtccgggcctacagtttgcatatggcggaagaactcatccttcttggtcaccatcgatacaacctgggggttgttaaggaagatgggggccaggcgacggagacgttcagcctcttggttctgagtaccccagttgataaccatggagccctgacggccacggtaggtagaaccctcggaacgaagaattcgtccacccagggcattgcgaagggctacagcaccattggatgcacggttgcctttgtagatatagatcatagtgtatctcctgtttcgttgaggttagtgaagccagcaatagcccctgaggtcctaagttgaaccccatgtacacatcttaggaaccggcatgggtcggccaggggctattactaacgcactaccttactcgaagtagtactcgtggagcatatccaccgcttcggccaggacgatcggggcaggcaccaggaattcttcccccggattagcctcattcagctcctgctccttcagccgacggatatccgccatcttatccagagtctctttggagaacggatacttgcgggttgcgtcggtttccatgtggattactacttgacgtttgaattgctgggtcatgtgacctccttatactgcttcggaaagtccttcacggacacatttgccacagacgaactcacactcgttaagctcgccggtgaacttgctaccttcgtacaccgtaagatcacggttccgttggtcataggctgtagggatatccccgcagacagcgcaaccacgacatacaaagtcttggaagacgtagctgttcacgaaggtatgcccttggcacttcagtggatacgagatattcgccggtggagtattgccagcctcggacttcatctgaagaactttagaaggggatgtccgcgttccaatgatctgcaacggggtcccatccaccggactggccttgtgctttgggtcaaggcaacttacggcaacaccttcgaccacaactttaaaatcgcgtccttcaccgatgaagtaggcgttggagatacttccacggagggtggtgtatccttcgagggcttcgacgttgagtccgtaggcttcgacgaccattccttcatcgaggtcgatcccgacgactgtaccaaagccagggtcggccttgtagggttggacttcgacgattcggaaggtgaggaggctgtgatgcttacggtagcttcctgcggatcggagattgttgttgattcgcagggtgttccttgcatacgcctcatccgaagccgttcttgacgacgaagctttttggccagcttgcgttcccgcttggctgcctttgcttccttgatcgtcatagtactccccgaggtattgcttccaggcgtctgccccgacatagttcccccagaacttttctgcctgacgataactttctggaatcggtaggtcgagaaaggtgaccggagagctgatgggctctacacccttccgcacgccggcctccgtgaacgggatggataccagagtttcttcagccagctccacgatgttaccgcgaagcttgattttgctggatgcacgctccaggaaggacttgagcatccaatcctcggatgcccatacgatggtccccgtggtagtaacagcgaagaacagaggtcgttcagagttacgaacgaagttcaggctgcgctccttggcatcccaccagaccagggcagcagcacccgagaagcgttcctccagggtgctacgaatcccatgcttggcgatggtggctgcaaccatgtgggaatcaaccttgagggtatcgttaccctccagcagatcgataccccaggcatccacggtgccattgtgaaccatcgtgatgtggtccacttggaaggggtgagcattctcatggtcccctttgtcacccatggttgcgtaacggttgtgtccgatgtagatgcgggcacggttttccgtgagaaacttcttggtatcctcgtgggccaggaagtcgtaggcatctacagccatcttgtggatactgacctcgttcttgatcggatcgaccttggccaaacccgtggcatgggcaccacggacgatatcagccgccaggaggttcttgagaagagcgatttcattatcactcgcgttagttgtagaacagaagcctactaatccgcacataggtgttactccttagtaaagttcttggtgaatgtttacgccgtaggtacgggtgacgaagttggtatggtcggtcatcagcataacgaaagcttgtacaacctgagccgctaccccctcgtcagtgagatcgaagacgttacggacggttgcatcctgataacccataccacgcagggcggtcacggtcatcaaggcattacccaggttagatacctcccctggagtccttgcgatgttgaaccgattgacaccctcctcagagaagctcaaaggagttgtcgctgccgcagtgagttctgcacgacggcggttctgttcagcacgctcttgttcacggatgcgccgctgacgatcctgctcttcctccatcgggtcaacaccattggcgatctgaatacccgtgctgatagccaaggcagcagccgccagacccatctcacggccacccggagacatgttgtagtccggggagagccccaggtggaacacatcccgcagatccacattggtcagtacctccagaaactcagaggtgtcaaccccttcgtgaccctgggcgaaggccttgaggcagagcatacgctgcatcagcccgaccatttcatcccgagaggtcatcgcatgggagccccggaactcgatggacccgaaggtcgtaacggtctggaggttcagggcggagtacttgggccagttcatcaggcctcgaatccgaagaccgtcttcattgacagccaggagacgacgaccgaaggtttgagccagccaatcgttggagaagaaggggatacagaagttgctatcccggcggtagggttcacagtggtcgaagaacacatcctcgaacaccatgtaggtcaggacggtcttgatgagaacatccatcgatgagtcccgcatgtccatgtgaaggtgggtggagcagcggaaggtgggtgccggcttgacagcttccatggtggcagcgaaacggtctacccgttggattgcctctgcaccgctcgaagggccatcgaagatgtactccataccactgcggagggaaccatcttccttgcggttccacccatcgatacggggccattcatcggaaccctccagttcaacctcgaacccgaaggacgcgattgctgggagttcctcatgggcttggatctcaggaagatccctcttgcggtggccaatcagagagtatagagccgacatagcagtccctcctgtttcttgaattcatcattgacaaccagttggccttccaccagattacctaccaacatacccttccagaagactcgcttgccttcctcggtgtagaagtcccgatgggatgggctggagatcatgtctcgggattgcaggaagatccggaggatatgcaccggatacaggctgccattgctgccaatcccgttcaggatgaagttccgggggtccagacctttcttctgagagcggttgttgacagcgaaggtgacgtaaacattacggaagcccgtcccgtgaatgtcatagtacccgctggccggtctgtcggtcactacatcactgaagcgaacccgagaatatcggccctcgttgtcatccagggtgtaggtacgtcccaggagaacgaacccatcgaaccgggtatcgtcgccatcttcctcggtgccctcgacgtacatcacatggggaacaccatccttctcgaagaagacataggtgtttccgtagtacatggtgaagtcgccggccagcggatgatcgttgttcaggtcattcatgagccacccccagcttctccagggccgccagagctgccactgcgtcgttctcgttgatgatacgttgtacctcggctgcaccgcctacggctgcgatggcgtcatccaggccgccttcgactaccacggagtaagccttgagggccgagtcgtatgcccacttaatcaactcatcggagaagatccagaagttggacaaggtacgatactcgacaccatagggcttgtagcggcaggcaccggccttaccgtagagcagcttacggtcggtgtcgccgtcgatcagcaccgaggccaggcccaggtagatatcgcaggcttggatcaccgcacggcagttggccttggtaacaggcaggtcgtcagcgaagccgatgtggatgtgaccgccagcggtacgaagggtcgggggtgcatcggtcggcatacggttacggctgcgggtccaggcgttgtagtccggctcacaaccgaagacgaatgctgcatcaccgaagccacggagaacatcttcctcgtagaggtgggaggacaggttgtcgatgacttccaggccgaaggggcgaattgtgtcgcgtccgatgttcaggacgctggtgatgccgtggtggaattgctcgaaggagctggccggatgaatgttgtactccagcagaacgttatcctcctgcaacgccccaccttccaccgggatgggctcgtctttagtaccaccgacgaagccgatggcagatgcgggttcacgaccgaagcctacgaagacttccgggtcagcaccaatcagagggatatttttcatggcagttctcctatttaatcagttggttttcggtgaagaatttaatgagggtgtcctcgtaactatcgagaggcattacctcatattgctcacggttcttgtcgttgaggtcgtaagtgatacctgcggacacgctgatgatatcatcatcctcgtacccatccagaccgagttcctcgatagctcggcagtctttggtatacccctgccagagagggtccttgtggccgctgtagagacgcttgaaggtttcccgagtacgacccatgatatcttggatatcgtacagggtgggcccgtggagaagctctggggcttgtacataatcaccccagctatcaatccgcccaaggcccaaagccttggccatcgtctccgtcaggtaggagtcatctacctctatgccctccttctcgcattgctcacgatactggccagcagtcacggggatcatgctgacgtagctgtcgcaatagaagttgtggccaatccttgcgcaggccacgaggacagcaaccacggggtccagtttctgtagcaggttcggctgatggctgctgtggaccagcatgtgggccatccgcagggcattgatgatggggcctccggttttgtccatatctacccggacatagaaacggctcatcgcccgtgggagttcgaagtccccgatgggcttgagatcaccattgccatcccaccactccttggggagatcctcggacgccgccaggatagcaccatcggtcatccggttgacgaaatcactgttgaggaagtccacagcgatccttcgggtcacttcgttggcagtatgggaaatgaagtggacaacgcagcccgcttcaccattcctgtcctcgaagtccccgtagcaacgggtatgaccattgaggttcttaacttgggagaagggtcttccctcccggtcgattacttctgccgcatacattcccatgattaatctcccatgagtttcttgaggtgttgacagattgcatccagactggggaggaaggactcttttcgtgccccattcatcggacgagacaggtgagggggtatcaactcgatacgctcccccgcgttcttcacggagggtacgtgctgaggaagcagcttgttgctctggaagaagggccagtggctatcctcccatttccccaggtagcagggctgagggtagatggtgatctggaactccttgtccatcgtcttccgcataccctcgatcttctccttattgaaaccatagtaagtaggagtaacccccaggatgaagtcccaggtgccatcctcctgcttcaaccaccggccataggttccttggtcttcgaccttgggcatttcccagtcgcccttgaggagtctttcgacggcctcgggcttccacaccttgccgtggacgtttctcagggggtgctcggtgtacccattggccatcaggttgtagcaggacattgccacgtaggataggaggaatccgtgcttatcggccaactccagatgggcctcttcctcccaatcgaacgcaccagaggatacccacttgaagaggagtttgaccacgggataccagacctccggggtagtggtccgggggttcatgtagataccgcacacgtaatcgttaccccacatgcggtcatcggtgaataccttctggttggggaatcgattccagggtgcgctcttggccttggaccccatgcgccagtggtggatcttggggacttttaccttggtgttggccaccgtcagggtaatgtcagcgaacatcttgttcatctggcggatgtacttgggggtacggcgagcttggatgtagaaccaaccgttccagttcgagaagtacttagcctccagagcgccaaagcaggagctactggtcatcatgacgagggagttcccgtggttcattgcgtacttgagtcgagccatcttgtttctccttggttgagttagccagcaatatccccagggagttcctagggatattactaacgccctcttagaaggggccgcgacgctgctgcttacccttgtggggcgcaggaaccttggtggccttgtagatgccgccattcagggtcagctcgccggaggttttcagtcgctcgtagaagactttggctttcataggctgtatccttgctggtgatgaggggagtaaggaaggccatgcacaggctgatgaagaggactaccatcaggaggtagtaaacccaatcatcatccggtggcctcttgggggatccagagattgctgggatattgtagatagcgtcatatatgtccgaagttacctcactggtacggtaacaccggcggtgcccttgggtgcggtccccattgctgttcgtcatagtacatcagacctccaccgtgaaggtatggcccttgggaagtaattcgaagatatactctacccgccctggttgaatgtcccaactgttgccagtctccaggcatacgaagggaagcgtctcggtgtcaaggttcagtctgcctgacacgaagattcgaccaccccctcggtgacgccagaagtgggggactacttcttgggttgtacgaacctttacttcatttttcagtcctgccaattcatcacgaagttcctggatctccatctccattccgccgcactgatggcgagcatcatcgcccttcgctgctgcgtcgagcgccatcgccaatgacttctccaattcctggatcttggccgtggctacttccagacgcttagcgtactcgtcgcggtcatacataacctgcatgaacttctcgaaactgacgatgggtttctccgactcactcatcatccacccccaaagtcttgacctttttctccagtcgcttactgtagttgtacaggtacttcaagaagaagagcccatagaagatggatgcaaccatcagaccggcacccagggcgctagccgaggttgaatccgaggtcatcaggtaggggaggatcagaaggtagagaatccacagagggatagccaccaacatagtccgggtatagagcttgagcatttgatacctcacataataagtaagatataaaggtagaccttaaggtatcctataggtataagagcaacccccggcaccttaaggttattataccacacatccagcagaaagtcaataggtcaaatgttacaaacctttaaccaacttgctgatgaagtcctcgctcgggatggatccgtggtcccagtcgtagcggaccaggttctcccggcacagcttgccccaggtgtcacggacccggatgatgtggactcgcacgagggtgtcgtggttgtcgctgctggcccaagggttgctggtgctccccaggcgactgaggatacggtcggcgacccgctgctcattgttcagggagaagaccatgagggcctcaccttcgtggtactgatcggccagggcctggataacatccattgcaaacttcttggggctgaacccctcggagtccttgatcgattggtagtcagcggtactcgtaggacccatgccggtgtagattttggcggtgcagcacgaggggaagttatgctctctcatttgatggctcctttgataattttgtagaaggatatgatgaagagtttcatcgggaagatgaggtaataggtcatcaacccgaagaggatcagtgggagtgataggggccacaacccgccaatgacgatggcgtgctcttccttgatccttccgctatcctcgaatagggccaaggtcacgcacactcctacggcggccacgaggaagtagatgaagaggcacatcatgaggaagaaggccgccgctgcatagacgattacgctttccattagaacctcctgtagtggccgctgaagagccgcttggctgatatgaagacttccttgaggggcctgtagcaggcgaagtacaggatcatccccggcagggacaagggccagaagaaggacaggagaactgcccaatcacgatctgacccccacctcatcccgcactcttccaataccgcatagatcaggcaggatgtgaacccgtagaccgccaacccgaagaagatagtggcggagatcatgaggatttcttggatggtcatcaccaatccctcgcgcagttgcagcaacgaccctctttgggaccgaagactggcatacagtgggggcaatcctccccggattggatcatagcctccctggcgtgccgcaggtaagccgatcggctcgactccacacctacggctgccaaggtgaagccaatctcaccacggggctcctgggggccttcctgacgggtcatgttgaaccctcgtttctctactcgacgcttcattcgccgctcctgggccatgatttggccgatcattggtttactcttcatacggcccccacatcctggctgacacgtggacttccttcgctgggtagagttcaccatggttgtataccacattggctgcgaagatggagaaaccaaccttggtgataatcgtcacggtcccgccttgtttgaggtagaccgaatcgcccgtgaggcttacatacacaccatctttacgcttactgatgtgcaagtcgctacgtggctgggatactgctacggtgaaagtagtcatgggtcacctcagtggacccgatgaaccttgtggtccaccgggatgccgagctcgtaaagggtttcggtgatgaactggccgaagttgatgaccttcaggtggaattcaccatcggggctagccagccagtaaacctggggttcagtggatttcatagacgatacctgcacgtttgccgaagatcgacacacggatgtgagtaacagaccagttggggtacagcttggggttaggctggtatcgccggatgacaaccttggataccaaccagtggtagatgtattgggcgggcgtcatagtacacaccctaggccggaggtccgaaccgatggacttcgatggacgcagccttgatggtcacggggtagaacccgcagtcaacatcgccgctgaagtatacggcacccacgggtccaccgaagatatccacgatgaagaattcatctgcaacttccatgtacataaagacacgcgggtttgcagtcccgtcctcgtcctggcggatgtacagacgaccagactccagatcatcccaagaggcaacccggatcttactggtgagcatattgtagttgagtttcatgttacacctcgtagcaattcagctcggcagttactgggtataccagactcaccggaggcatgtaaatgacgttgccagtcttgagattgagaacgggaacctcgtcggattcgcccttcatcttaatgctgcggagttcgcagaccatgtacacatccccaccgatgaggaatgtttgggacttaaccagagaactgagggttactccgttgcggggccacaccttgatatccatggggctacctcacgatgcaaaagggatgctgtccaaggtcgaggacaggacgaagagaaccacccacactaccagcaacaacccattggaacgtgagcgtatggcggcacatgcaatatacttccatgccatcaccgcgccctgttcttaaccaaagatgcccgcttggcacggagcttgttcaactccagggttttaccaacgtggtttcctccggagttagtcaactcacggacagccttggcaatcttgcggtcgatagcctcgatggcttgcatgttgcggctaaccttacgatagtcgttgcgtttcacgggatagtttcctttgttgatttcccaagactccccagcctgcgaccataggtccggtgggggtttcggccaggaatcaccatggcctcatcagttgggattagatacccatttcctctttccaatcacgaaagaagaagtaggcgataccgatgctggatacaatgatagcgatggtcatttgctaagtttccttatgagttcctggcgctcaacaatccaccggggatcgggatgcacccggatcatcctagtgagtttcttgagacggttaatcatttgggcacgttcatgggccgcttcgtaaggtgacttcatttgatccacccgcagatgttcagacggataccttccagagtgtaaacaccgcaggcaaatttaccgtccttgaatgtccagcggtacacgataccagcgtcgaaacctacccttgcatcgcagaaaccagaaacatagtcctggccttggatggaatgaacctgcccaaagatacagtaacccacggaaacctcctgatggtctatcgctacaatgcacggacctaccatgcactctagggatagacctggggcgcgtcatagtacacaccccagtcgcacccgccgttacgcagccttcgcttcgtccttgacttccagtcgctggatgatggcgatacgctcgtccgctgcctggagctggcccttgtcgatggcgcgttgaaccttggccttgaatgcggcgatggcttggtccagggagaactcgtcggccacgtcacgctctttcttgtacttgaaccagggtttttcggctgcacgctccagtcgagtggccttgtccttggcgaagacgaacgggaattccttggccttttccttgtcggtattggcctcgatcttgccgttgtctacgaaccagtgggccagggccagccggcgggagccttgcggcatcgcgttgaacagcttgcatgccaaggacacctcgccatgctcgtcgatatgcttgaggatatccatgccggtggtttgaaccagcttgtccagggcagcaccccggttggcaatggtcttgatgatcccattgatacgcttgtagtcgctcattgtatttacctcagttgtttacttcgacagtgatgacggcagagttaccacacttgccaggcttataccagttccgaaggttagtaacctcacggttacgccaatcggtactcataagagccttgcgtttacgcgcacctgccttttcttgcaacgaagtggtcttgtcgtcgaggatcttgttacaacttgcaataatcccaaacttctgctcactacgctgttgggccttccccagattcccaaggttgtaacccaaagccttggccttgcggttggccttgcgacgcagctttttgtcaagagcctgagtcatggtcgtttcatcctttaggaatcatcagccgggacacacatcccgatacgacaaaggggtttatcggcaaaccttggcattggcgta

SEQ ID NO: 6 - DNA, 66116 bp, Pseudomonas aeruginosa phage
gtgattactcttcgccttcgtccgcgctcatccactcttcgaaggcgaaattcactttcgactcgacccaatcctgcagctcatcggcgaactcgtcactgtcgatatccatcggaacgcccagagtttcttcattccacaattcagcattcagctcgaactggaccatttgctcgccatcgacttccatcaggatgcggtctgcgacttcatagtcgtctacatcgaacagatggccgccagtcaggaggtgctggacgaaagcttcatcgagattggtgacttcgatctggacttgtttggtcattttccgtctccttacttagcggctttgaattgggctttgaggttggccagttcagctttcagatcgatcatcgcctgaccgcgcaggccttccatttcggcctcgctgatcgcgatcttggctgcgcggattttgtcgttgatttgatctttagtcattttcggcttccttttgtttgaagggttttgcgtttcaatgaggagattatgccgaaagtcagaatggaagtaaagcatcttgatgagaaaatttctgcccaccgacgaacggtcgtgctcgaccgtcgattgagggtgtgcataatcgaagtcgcacgacgcgaccggtgatgccccggcgaaccggggcggggtggttaggcggtgcgctcggcggcgcgttctgcgttacggtcgtgcgccatgcgcgcccatactgcggcccagcgcgctttatcggcagagttcggcttgtacacgtcgtcagggggcggttccgggccgtaggcgttgcagctttcgtcgtattcgtaggtcatcatcttgccctcaagggggtcatggcccggcgaaccgggcgggtgattacttgcgatatttcttaatcaggtcgtgtgccatggcgatggcatcagaataggtttctccgccagcctccatttccttcacagccttattgcgcagtgcgttcagcttgttgccgttgatggtgcctttttcatccttggcagcttcgcaggagtcgcgcagagttttcagggcttggtaggtggcgttcatcgtcttgttctccgggtttcgcgtttcgatgaggagattatgccgatagtcagaatggaagtaaagtgtctgaatgaagaaatttctgaacaccgacgaacggttgtgctcgaccgtctgctgcggtgtcgatatactcggcctattgcgaacaatggactgattaaatgttcaagctcaatcctgcactgcgagcggtctggcgaactcgtgcccgttacaaagtcatctatggcggccgggcgtcttcgaagtcgcacgacgctggcggtatagccgttttcctcgcggccaactacaagctcaaattcctctgtgctcgccagttccagaaccgcatcagcgaatctgtctacacattgatcaaagacaagatcgaaaactcagagtataatggcgaattcatcttcaccaagaactccatcaagcacaagattaccggctcagagttcctgttttatgggatcgcccgtaacctgtcggaaatcaagtccaccgaaggcatcgacattctctggcttgaggaagctcactacctgacccaggagcaatgggaagttatcgagccgaccatccggaaagaaaactctgaaatctggatcatcttcaaccctaacgaagtcaccgatttcgtgtatcagaacttcgtggtcaaacccccgaaagactcctgcgtcaagatgatcaactggaacgaaaatccgttcctcagtgagacgatgctcaaagtgattcacgaagcttatgagcgcgaccgggagcaggccgagcacatttatggcgggattccgaagactggaggcgacaaatccgtcatcaatctcaagttcattcttgcggccatcgacgcccacaagaaactcggctgggagccggccggatcgaagcgcatcggcttcgacgttgcggacgacggcgaggatgcgaacgcaactacgctcatgcacggcaacatcatcatggaagtggacgaatgggacggcctggaagatgaactgctcaagtcgtccagccgcgtttacaacctggcgaagatgaaaggcgcctcggtcacttatgactccatcggcgtcggcgctcatgtcggttcgaagttcgccgagttgaacgatgccagccccgacttcaaacttatctatgatccattcaacgcgggcggcgctgtcgataagcctgatgatgtctacatgaagctgccgcacacgacgatcaagaacaaagaccacttcagcaacatcaaggcgcagaagtgggaagaagtcgcgacccgattcaggaagacttatgaagcggttgagcatggaaaggtttatccattcgacgaattgatttcgatcaactctgaaacgattcacccggacaaactaaatcaattgtgtattgaactttcgtcgccgcgcaaagacctggacatgaacggccggttcaaagtcgagtccaagaaggatatgcgcgagaaacgcaagatcaagtcaccgaacatcgctgactcggtgattatgtcggccattctgccgatccggaagcccaaaggcttcttcgacttctaaacatagaaaagcccggatcgctccgggcttcgggtcttactcggtgtggttcctggcgctgagtgtcgacgcaacggcctcgccgactcccagggcttcctggccggccgcgagcgcttcggcttccgactcgacgatgaaatcatcaccttggccatcgcctggcggaacctcgaccagcacggcttcttcgccttcgaaccgcaggtcataggtcttttcgacggacagaccgtaacgagcgttgagcgcgtcccagagctgggcctcataggtccgcaggtcttgcagagctttctggtggctgagcatggccatgtcaacagcccgttgcagggtttcgtccaggacgttgaatcgcatgcgaagagaacgaatccgctcgaccacttccgcatccactacatgcctttcgatcatcgcttttcacctttgctgaatgttacgttgtagccgttgtcggccagataggtcaaggcgccttcgaaagaagttccgacaaggtgcttgagctgcatttcgcgttgcgcggcgatccagaatgccgttccggagaactctgcgcggccttccgacaaaacctttccatcaggcccgtcgatccgtacatggacggaagatagcttcagcgccatcagtgaaccgtccctgtgaaggcgcgcgccgccgcgcattgatcgcaagggcactccctctggatgcgcgccaggtcgtttgcatccagggcgtagagttttccatctacggtatcgtgagccattgcgatgtccggatattcgttttcgtccaggccggatgccgcacggatgttgttccagatgccggcatgttccgcgcgatggcgcgcggtcaactcatcttgctctttgttcgccctttcaacgaatttctccaggagttcggtgaatttctcatcgatgactcgtgcagacttgacagagctgagtcgaatcgggtctttcttcatggtgattctcttttgctggtggttgttcgctgcccagacctattcaaagtctgggcagcatgatgaaaatgaacacgaaggctgcaaacgccaatagcgttcctgtaagcattttggcttgactctgcacctcagcgtactgcttagtggacattccgtgctgcgccgcctcggccgcgaagcgagctttcgcctcgataacttccgggcgcagcgacaggacatattctaaggcctcttcccgcgctttttcggcctcgaccaacctagggtcgcgggccgagacttcgctgtgccctggcctcgcgggatgggcctgcagcgatggcggaagttcggcggccacgactccatagtcggcgcaggcccaagcgatcccgatgaggatcgcgaggatggactggacgattcgcaacatcactttgtcgctaggaaagctcatgatcaatcctccaccgaccgaacgatttccatgttgcgaccggcattggcccctgcattgaaggcgcgccgaccgtcgctatcgtccagacgcatgagcttggtgacatccgacttcttgtagccaggatcgccgaaatgttcgtgaaccgcagcttccttcaccacaaccagagacgttccggcagaagataccagctccatgcgcttcctggttatggactgaaggcgatagctgatttcctgggtcgcggccagcttgaattgcgcggcaacctttacgttgaaacgttcgtacccttgcgccttctgatactcccgacacagacgatcaatggcctcgaccagggagttgaacatgttcaccgccagctcaacgtccgacttgtagcctttgaagcgtacggcatgaccccagcgcttggtagtgcttccgtcgcgggcgctcctggatgccttagccgatgctctgtggttgttgatgccaccggcgaaatccatgatgcagtcattgtacgtcgccacggccactgagaaaaacttcatccagttcgggattgcggaatagtaacgagtcgcaatctgctcgtcgaactcttcacgaatctcgccggtcgcttcgaagccgtggaggtcatacttgtccttcagcttcttcacgcgctcggctgcgatggccgcttcgtgcgggctggaggagtcggctgccatggcggtcagtttgcggatgcggtctttcgccttctcgatggcttccggggtgaattcgttctggtcggtcatggtcggttccttttgtctgaagggtttcgcatttcaatggagctattctgccttcattcagaatggaagtaaagcactttcttccactatttcggcatcgactggaagaaattccagatccaatcacctgctaccagcgagaggatgagggcggcaaagaacaagacagcggccgcgagctgtgcgcccggcttcagtttgggatggctgagcttgggctctacgggaacggccggagcgctagcgctgcaaccggcgtctcctgggccgtagccgatgccgcgttcacgcgcctggagcgacaccagggatttaagatactcggtctgcttttgcgactcttcgtagatgccggcgacggcgaaccagagggcgaacactacggacgtacacacaacccaggcgccggtcagcaggagggccagcgggccgatgacgaagacggtagcagccaggacgattgcgccgccccagatgatgaagccggccagaccattggtgatgtcgatacagaactttttcattttcagattccttcggttacgggatggaggggatttgaaactctgcgccgccgagaacatcaatgacgacttcccagagcgtcggaacagaccactggtaacggtcgaagtcggtttcaggatcgacgcccagcgtcacataggaagtggcaggccatttatgaacactgcctttccgcatgagagtcgccacgcggccgtcaccgagcggattcgtcctgtgcggaacgtacgcagacatcgaatattgctgcccggcgtcgacaacaaatctttgcctctggcgcagacatacagagcccacaggattccagccgcgaccgccgcaagactggcgagccgaagaatgctcataaccttcgtgagtcatttcgccgaggcaagccgagcgatcctcgtaatagttgatgttctccatcgatccaaccagcaccagagactcgaaatcgaatcgatgatcgtggatggccgagtggttgaagcaaagccgacgcggcagctccgggtgccaaacatggaggcgaccggccggaagttggacctgaatgaaccccaggccgtgcagagtgatcttgtccttcatcgggtcatggacggtgctcatggataatcctcagtagcagaaatgtattgtgagagttacgattgataagccggtcgcccatagaagggcgaaccaggccatggctttgatggtcgtgtagatcatccgaagaattttccggcacagatcgggccaatgcccatttcgatggatgcgtggttggtcaactcgcgaccgcagcaggagcactgaccagtcttccgaccgtaggcgactgccgattccatcggcttttcgaacatcttcagaacgtcgtcgtgctcagtatcggtgcagtcgcgactcttgatgaatttgccattggtgatccggccgaggtagatgtcgcccaggacgtacaggctaccggcgttccggctgtgagcgctagcctctttcacaacaacgatgagcggctcctcgccttcgccagctaggcggattttcgggcgcttgataccagactctttcgccttctcaaacgctttctcgatgccggaaatgtccagagtcggcgcagcagcttcctgcgcggccactttctcgcgatacttggcgaggttttcgatggcgcgcttcgcagcagcgatctgattttctgtcaaggagccgtatttatacaacgactcctgaaggctctgagcgaagctgaaggaatttccagtccaccactcgatgatgtccgggtgcgcggcttcgaaggccttaattttgaggtcgcgctcttgcgcagcgctacagattttctcgatgcgcttttctgccgccttagcacggctcttggcgcgctgctccgggctggttttgtactctttgtatccgacgccgccgcaggcaaagcaggcgcgaccataagacgaagggccacggtacaggccggtgcctgcgcatttggtgcacttgtcgcgatacagcttcacttccttccgggagttcgggcgggcacccatggacacgtcttccagggtcttcggcgcttcgttgttgatctctacggtagcgaagtcatcacccaggtcttcgaagccggtgaacagattctctgctgcgttcatgtcgattctcctgtttggaaagttcgtttcgatgagttgactatacgccagaagtggaaaaacggtagcgatttctcactaccgttcgtcgggtcgaagacaatcaataaatgtagctattgatcttgaacccatgctcagcgccgtcgttgtagtcgtactcaacataactgtcgcagtagtcgttcaaatgctgaatgatgcctagaacgtcgttcgcggccttgtgccttttcgcggcgatggtcttggcgatgctgtcgccgacttccagggtttcggccgtccggcgatggatcagcaggcgactccaaagatagagccggacgcgacgaatgatgtcgcgatggcgctccagtcgtccgtgcaactcttcgatttcgtgctcgcgaaattttaatactcgtcgaagctgttgaacttccaactccaaatcggccttagtagccatgttcacctcagaaaggataatcgtctgagactccaggaagctcgacgattattgttgagcctgatcggtctagaatgcatccgaccgagccagccctgtgtggatagggtctacacgaactataacaggtgacttccctgactattcgccaggtcgatcctccacaccatctgcaaattggcgggcgcttcgacttcagttttcgttcgtccaggacggcgcgtctgtcgcaggaaaggcagcgtgccttaaccgacgtaaccatgggtatagtcgatcattcggatgagctcatcatcctctgattctctcgacttcagaccgcccaaagagtccgactggccgtcgcatttcgcatcgatatccatcgaagacaggaatcctgccgatactgcggtgatatcaactttgtcgccggccttcttgaagccttcgcactcagcatcgagggccacaattgcactcgcggccatgttcacatgactgatgaggtcggggaagatcacaggaacttcacgcgacatgccacggaccgtcagcttcaggactacatacttcatactcactatcccttttgtatgtgaggaaagaacttgctgttttccggatggtgaaaacgctcggtcgcaggcggtctttcttccggacattgaatcgtcgaaggcgggaaaaccgcgccggcgataatcgccgcgaggagtgcagcggatgtcgacatccagagggcggtttccaggctcacccgaacttccggacggcgcggcttcattcgctccaccctcttcctggctcataggatggcagaccgccgccgcgaatccaattgtcccacatacggttcagaccagtcggtggctggggcggcggactgtagaatccgggagtctcagtgctgctcaggaaataaggcgtgcaaacggcctggacgaccaattggtgccgctcccacatcttatcaatcgctcttagcatgacgtcttcgcactgagccttactgtcgaaccgtctgctagtatggtccggcatctggacgcagccgtctccagtgcaaaggaaagcagtagcaatccatacggtgatactcgccatttcgtcaccctctttagttgatgagcagagtctattccatctgctcgccaggagtaaagcgcttttcgtcgggataaatgccgatgatgtctgcgtcgagcatccaaatctccacggacggatcgtcgctgctgatttgatataggttgtacagctcccgtccgccgacagcacgctcgcctcgtggctcaaccgccagaaccttgccgtgtccttctccgtgctcgtccaggtatatgacgtgatcaccgacttcatagctttctttcgtaacgaggcgtgaacgcgagctgttctgcgattccaccatccaggaatccagcacgccgcctttcatgtcgcgaaacaatcttgccgatttgtcgcaatcgaatacgcccagaacttcgccgtctttcaacacgatatgtacgattggcaaaagagtattcatgtttaatctccattggttgataattagagtctaatctgccgaaaagttcccgtaaagaattattttctcataactgattagttgcaactgttaatctgatgtatatgtttgaatctcttttgaacgtttgatgtttcccctataataagcgcacacagccaacaaccacgtggaactacaatgtttaaactttcctggatattcgggcgcaaaaaggataatgctgcctgttctgaatcggcgccggagaaagtcgcacaaatccctcaacacgatccgctcgaccccatgataaagctgggaaggattcgcggctggaacgtcgagccggagaaagccccggtcattcgtagtgtgaaagatttcctggagccgggcctatctgtcgcaatggatagtgcgtatggtgatggaccaaccccggccgcgaaggctgctgcgggcggccagaatccctatgtcgtcccgactatgttgcaggactggtacaattcccaagggttcatcggataccaagcttgcgcaatcatttcccaacactggttggtggacaaagcttgttccatgtctggggaagacgcagcacggaacggatgggaactcaaatcggacggcaggaagctatccgatgaacaaagcgcgctgatcgcccggcgcgacatggagtttcgcgtcaaagacaacctcgtcgaactcaacagattcaagaacgttttcggcgttcgcatcgcgctgttcgttgtggagtctgacgatccggactactacgaaaaaccgttcaatccggatggaatcacgcccggctcctacaagggaatctcccagatcgatccatactgggcgatgccgcagctcactgccggctcgacggccgatccgtcttccgaacatttctatgagccggatttctggattatcagcgggaagaaatatcatcgcagccatctagtagtcgttcgcggaccgcagccgccagacatcctgaagcctacatacatcttcggcggtatcccgctcacccagagaatctatgagcgcgtgtatgcggcggaacggacggcgaacgaagccccgctgcttgccatgtcgaagcgaaccagcaccattcacgttgacgtggaaaaggccatcgcgaatgaagacgctttcaacgctcgcctggcgttctggattgccaatcgcgataaccacggcgtgaaagtcttgggtactgatgaaagcatggagcagttcgacacgaaccttgccgacttcgacagcatcatcatgaaccaatatcagctggtcgctgccatcgccaagactccagctacgaagctcctcggcacttctccaaaaggattcaatgccactggtgagcacgaaacgatttcttatcacgaagaactggagtccattcaagagcacatattcgacccacttcttgaacgccactatttgcttctggcgaagtcggaagaaatcgatgtgcagctggaaatcgtctggaaccctgtggactccacgtccagccagcaacaagccgaattgaacaataagaaagccgcgaccgatgaaatctacatcaactctggcgttgtgtcgccggatgaagttcgcgagcgtctgcgcgacgatccgcgttccggctacaaccgactcaccgacgatcaggcagaaaccgaaccgggcatgtctccggaaaacctggccgaattcgagaaggccggtgcacagtcggccaaggcgaaaggcgaagccgagcgagccgaagcccaggcgggcgccgtagagggcgcaggcgccccggttcccgccgctccacgcgggactaagcctctcgcgaaagcggccgaggaaggggccagcgaggccgctgaaccgccgtcgcggccggaccccaaggccgagctgcggaacttgttggtcgatcttttatcgaagctccaagacctggacgacattaaggcgccggacggcgtagacatagagtacaatgatgcgcctggcttaaagcgcacgtccaaacctggcgtgtctggcatggagccttcggtgttttcgtccaaccgcatcgtcgggcctcgtgatcattcggaactccagagaatcaaggtgaatggaataaccaccttgatcgaaaatccgcgcggaagcattcggcaaggaaaggatgggagttggcgagttcagatgaaacaccactacgggttcatcaagggaacgaaaggcgctgatggggatgaagtcgattgcttcgtcggtccgaatctgggatccaaacgggtcttcgtcgtcaaccaggtgaacaaggaaggtcagtttgacgagcacaagtgcatgctcggcttcaacaacatcaacgatgcgaagtctggatatctgtcctgcttccgcccaggttgggatggtctcggctccatacatgaagttgacctacccgcgttccgtcgctggctggcgaacggcgacacaaccaaaccgttcgggggcgagtgatggcattcaaggcctccaaaaagcgcgaacgccgggcacctcttccagtcggaagagggaagcccataattccatcggcaggaatcgaggcctggtatcgaaagcagatgaaggatatgtccaagctcatgatcgccgactatcgaagcgagattgagaatgcactgtcccagcctgcggccgaacggttcttcgccagcgacgaatccgttaacgttctgttcaagatgacccttcgaagcctacagcagcgatggagccgcatttttgaaggcttcgcggccaagatcgccccggagtttgttaaccggaccgaagaagcggccaccgccgcgaccctgcacagcttgtctgtggccggcgtcgatcagccaagagctgcgtacaatgagagcgtcaggaataccctggaggcagcaactacttacaaccatactctcatcaccaggattcaagaggaagtccacgagaagatttacacatcagtaatgttgtctctgacttccccaaatccggaagagcaaggaacttccggcataacgaacgcacttcgcaaagtcgggaagttctctgaagatcgaatcgaactcatcgcaagagatcaaaccagcaagctttacagttctctgagcgatgagagaatggcggaaaatggagtcgaggaattcgagtggatgcactcttctgccggcaagactcctcgccatacccacctggagaaagatgggaaaaggttcaagctgaatgaccccagactttgggagggtccgaaggccgaccaggggccgccaggatgggcgattaactgtaggtgcagaaagataccaatcatttgatatcgataggagcgctatatgccgttagttcatggcacttccaatgaggctcgttctgaaaacatcaagcgggagatcgaagccggtaaagacccaaagcaggctgcggccatcgcttactccatccagcgcagtgagaaagggaagacggcgaaagattgttcgcctgagctcgttgccgatcttcgcgccctggtggacgctctgtcgaggctcgtgaaatgaaccgcaagacatgccggcgccgactcgtggtcgatgtaatcagggccaatattcacggcggattcttcagcctgaagtttgccgccatcgatttggcaatcatcggcgtcgccatcttgatgacttttggccgataatgctgagaaaatctggattctgactaaaaattctagtccggatagccgcaagttaccgtttacggaaaatagcagtaatttggaaagcctactgccgcgaggctttaacagagctagttcctaatttccgatttagccgcgtgcttcaaaagtatatagcctgggaaattagaagtaacgttccaatagaattcatctataagtaacgttataatataacgtcaatctatatgctctagacgtattgaaattcaatttttaattggtaaattggtaatttggattagtttaggagttgaaagtctcgcggcagtaggcttagacaaatcccgtaaagtttccgagaccaaattaccggattttcgcggctgaggaaactggtaattagatcataatataaattataatgtaagttaacagtcgcggctacatctaattattgttccgcttatttacccttagatgtactgcgtatataatacagccatagtccacgactcttcgaattaacgatgacaaagtcgaaaagaaaaattgacgaaaatggatatatgaccatcgagggctgcccgatcagctcttatggcgttttccagtattctgctggtcaactcggtcttccgggcgatccgacgcggattgtcaacgtatatcgcccggagtctgccgtcagcgatcctgagtacatcgaatctctgaagaatctcccgttgatcgacgagcacgaaatgctgtcgggattcgacgacgatgacgacagcgtggcccccgaagacaaaggggtggagggcatcatcacctccaacgcttactacgaagccccatgggctcgcggcgatatccgcatctattcccgcaacatgcagaatcagctggaaaggggcaaagaagacctgtccctaggctatagttgccgctacactgagcaacccggcatctggaacgggacgccttatgaagtcgtccaggacaagatgcgcggcaaccacatcgccctggtaaaagagggtcgtgtgccgggggccagagtattggatggtctgtgttttgaccatctcagttttgatttcagaccatccgatgagggtaatgaaatgagtctcaagaaagccaagcggaagccccctgtccagcgcgtagggcaagctgctgactcggcggtcgaagagttgcgcaccctgtggccgaaactctctgcgtctgtccagaagttcctgggcgaagaagagcaggagccggagcatcaggaaggcgccgctccggccgaaccgaccgacagcgagcacctgaccgagcatccgactctggaaggcgcccagaaggacgacgaagagcaggaagaggagccttccgttgtcgatccggccgtggccgccgtcgagccggagcatcaagaaagcgccgcatccgaaatgtccggtgaaggcgaagtcgccgaactgatctctcaggtcaaagccattctggctcgactggagggaacggtagccgaaggggcagacgaagagcatggcgaaggtcaagatgtcgtcgagggtttggaggagcagagcagcctcagcggctcgcaaaccgccagcgacgatggtggtgagagcaaggataacagcgaggaacttcctgaaatggcacagaagaacgcgcaagatgctgcaattcgcggtctctatcgcgacattgctgctaaagatcgcctctacaagcgtcttagctccgtggttggtgcgttcgatcaccgagctatggactcggctgaagtcgctgtttacggcgtgaaaaagctgaacatcagctgcgcgaagggccaggaagctctggcgctcgacatgtacctgaaaggcgtcgaagcctcgcgcggcgcggccagccgtcaatcgaaagcccaggattcggccggttctgctccgcagtgcgccgagctggacagctacctgaagggggagtaactcatgttccagaaacaagtttaccgccagtacactcctggttttccgggcgatctgatcgaggacggcccgaagcgggcgcggccgggtcgaatcatgtctctgtctgccgtaaatccggctgccaccgccaccggccccaaccgcatcagtcgcgctttcggttacgccggtgacgtcagcgccctcggtgaaggtcagccgaagaccatcgcggctcgtgcttctgaagtcgtgatcggcggcgccaacttcttcggcgtcctcggccatccgaagcattatgccctgttcggttcggccggcgactccctggctcccagctatgatctgcccgatggcgccgaaggcgagttcttcgacatggccaccggcctggtcgtcgaaattttcaacggcgccgcagccgccctggacctggactatggtgacctggtcgcctatgtccccaacaacctggctaccgccgacaacgcgcttggcctgccggccggcgccctggttggcttcaagactggctccatgccgaccggcttggtccagattcccaacgcacgcatcgttaacgccatcagtctgccggcccagtcggcggggaatctggttgctggcgttaccatcgtccagctcacgcagtaaggaggcgtcatgagccagatcagcaagacccattcgcgcctcgcaggccgcaatgcgaaacctttcgacctgaagaacatcaccaatgatgccgtggcatctctgcgccgcatcggactggtattcgatcacgctgtcgtccaggaccagatcaaggccttggcgaaggccggcgcgttccgctccggctcggccatggacagcaacttcaccgccccggtgaccacgccgtccatcccgactcccatccagttcctgcagacctggttgcctggcttcgtgaaagtcatgaccgcagcgcggaagatcgacgaaatcatcggcatcgataccgttggctcctgggacgatcaagagatcgttcaaggcatcgtggagccggccggcactgcggtggaatacggcgaccataccaacatcccgctgaccagctggaatgccaacttcgaacgccgcaccatcgttcgtggcgagctgggcatgatggtgggtaccctggaagagggtcgcgcctcggccatccgtctgaacagcgccgaaaccaagcgccaacaggcggccatcggtctggaaatcttccgcaacgccatcggtttctacggctggcagagcggcctgggcaaccgcacctatggtttcctgaacgatcccaacctgccggcattccagaccccgccgagccagggctggtccactgccgactgggcaggcatcatcggcgacatccgcgaggccgttcgccagctgcgtattcagagccaagatcagatcgatccgaaggcggaaaagatcactctggccctggccaccagcaaggtggactacctgtcggtaaccacgccttacggcatttcggtttcggactggatcgaacagacctatccgaaaatgcggatcgtgtcggctccggaattgtccggcgtccagatgaaaaaccaagagccggaagatgctctggtgctcttcgtcgaagacgtgaacgcggccgtcgatggcagcaccgatggcggcagcgtgttcagccagctggtacagagcaagttcattaccctgggtgtcgaaaagcgggcgaagtcgtacgtggaagacttctccaacggcaccgccggtgcgctgtgcaagcgtccgtgggctgtggtgcgctacctcggcatctaacagatgcctattcaccaaaggccgggcttccggcctttgttcactctgactctaactcggttgtaggggccggttagggcataattcctaggactacgccaatgactgtttatatcgtttcctccatgactcaatcggtgtcttacaatgcgtatgacacctctgatccgtccaatcctcgcctccagcgaaagattctgattcgcggccgcgcaggcatcgcatccgaaacttccggcttcggcgacatgatttccgacgaggctgggcgtccgatctggaccccacagggcgattgcactgccgtgagcgattcggatttcgagctgcttcaggccaataagattttcatgcgtcacatggataagggctatttgcgagtcgtgaagaccgacatcaccagcgaccaccagcggatttccaaagagactcgtaccatggagcgcgatggattccagcctctggacgctactcgcctgcagcagaaaatcaaggtgaccacagccagcgcttcccaggaacaagagttccggatttaaccgagggtttcggcatggtgattttcgacgaaaataagtttcgcacgctgtttccggagtttgctgatccatccgcttatccggacgtgcgcctgcagatgtatttcgacattgcgtgcgaattcatttctgatcgcgattcaccataccggattctcaacggcaaagccttggaagcgtgcctgtatcttctgaccgcccacctcctgtcgctgtccacgatgcaagttcaaggcgcggcgggaggtggcgtcacagcaggtgggacccaaggcggtttcatcaccagcgctactgtcggtgaagttagcgttgctaagctcgcgcccccggccaagaatggttggcagtggtggctgtccgggacgccttacggccaagagctttgggcgctcctcagcgtcaagtctgtgggcggattctatatcggcggccttccagagcgccgaggcttccggaaggttggagggacgttctggtgatccctggcgcgaatcttctgcgaatggccttcggggtcatcggcactcaaattgtgagatatcgcaagtttgagcagcgagtgaagaatgatcaagcccagtatgtctccatgttcggggagcctttcgacctggcagcgtctgtccagcgagtccgacgcgatcagtatgcccagtttaatctggagttccaacggaattatgttatgatcttcgccaactttgatatggttgacttggatcgcaatatggccggtgaccagttcctctggactggaagagtttttcagctggagtctcaaggctcctggttttatcaggacggctggggagtttgcctggccgtggatatcggtgcggccaagttcaccgatgacgggaaaccgactttctaggtggtgtatgtttgacggcgaactgatagcgaaaatggttgtcgagctgaatgcggcgatggcatctgctcaagaggctttgcagttcccggattttgaagtcgtccagaaagctcagccgacccaacagggggcgtcaaccaggccgaccatctttttccagaaactgtttgacgttcctcgcggctggcctgccaccgattggtacctggacaacacggcccgcaaatatgtagaaataactcgacagcatgtagagacgactttccagatcagttcccttcattggcagaatcctgaaataactcacgtggttacggcttctgatatcgccaactatgtgagggcttatttccaagctcgatccacgattgagcgcgtaaaggaactggacttcctcattcttcgcgtgtctcaaatttccaacgaagcattcgagaacgacaatcaccagttcgaattccacccaagttttgacatgattgtaacttacaaccaatatattcgcctgtacgaaaacgcagcatattcggccgatggggtattaataggcatatgagtctgaggcgcgattcagagctaatcgccgcgcacctccagatgttaagagccatgcgcggcaggtccgtttcagccggatggtattccacggctcgatatcctgacaaggcaggcggatcggtcggaatacaagtcgcgagaatcgcacgtctcaatgagtacggcggaactatcgaccatccgggcgggaccaggtatattagggacgccattgttcggggccggtttgttggcgttcggttcgtcagaaacgactttccgggagaaaccgaggtaactaagcctcacagaattaccatcccggctagaccgtttatgcgatatgcttggaacttgttttccgcagatcgcgccgcaatccagaaccggatagccatgaggctggccagaggacagatcactccagatcaagctcttgcccagatcggcctggcattggaaggatacatagccagaagcatcaggaccgggccatgggtggctaactcagcatctacggtcaggagaaagggattcaacagaccgctggtcgatacggcgcacatgcttcaatcgattagcagcagagtaacataaccaggagatcatccagtgatcagtcagagccgttatatccggatcatttcgggcgtaggcgcaggcgctccggtcgcaggccgaaagctgattctgcgcgtcatgactaccaacaacgtcatcccgcccggaatcgtcatcgagttcgacaacgccaacgcagtcctgtcatacttcggcgcgcagtcggaagagtatcagcgggctgcggcttatttcaaattcatcagtaaaagcgtgaattcgccgtccagcatcagcttcgctcgctgggtaaacaccgccatcgcgccgatggttgttggtgacaatctgccgaagaccatcgccgatttcgccggcttctcagcaggggttctgaccatcatggtcggcgcggccgaacagaacatcaccgccatcgatacgtccgccgcgacttctatggacaacgtggcgtcgatcatccagaccgaaatccgcaagaacgccgacccgcagctggcccaggctaccgttacctggaatcagaacaccaaccagttcaccttggtcggcgccaccatcggcaccggcgtcctggctgtggcgaaatctgccgatccccaggacatgtccactgccctcggctggtccacctccaacgtcgtcaacgtcgccggccaggctgccgatcttcccgacgcggccgttgccaagagcaccaatgtcagcaacaacttcggttcgttcctgttcgccggtgcgccgctcgacaatgaccagatcaaggccgtgtcggcctggaacgcggctcagaacaaccagttcatctacacggtcgcaacttccctggcgaacctcggcactcttttcaccttggtgaatggcaacgccgggaccgccctgaacgtgctgtcggcgactgctgccaacgacttcgtggagcagtgccccagcgagattctggccgccaccaactacgatgagccgggtgcttcgcaaaactacatgtactaccaattccctggccgcaacatcaccgtttccgacgataccgttgcgaacaccgtcgacaagagccggggcaactacatcggcgtcacccaggccaatggccagcaactcgcgttctaccagcgcggcattctgtgcggcggtccgaccgatgctgtggacatgaacgtctatgccaacgaaatctggctgaagtcggctatcgctcaagcgctcctggacttgttcctgaacgtcaatgcggttccggcgagcagcactggcgaggcgatgaccctggcggtgctgcagccggttctggacaaggcgaccgccaacggcacgttcacctacggcaaggaaatcagcgccgtccagcagcagtacatcacccaagtcaccggtgatcgccgcgcctggcgtcaagtccaaaccctgggttactggatcaacatcaccttctccagctataccaacagcaacactggcttgaccgagtggaaggccaactacacgctgatctattcgaagggcgatgccatccgcttcgtcgaagggtcggatgtgatgatctaatggtttgcggcggactcgatccgccgcgaccttccataaatggagtgaggaataaacaatgatcaacatttctgcgttcggctcgatctgccagttcacggcaagcagaactttcccgaacggattcaccgtcaccgagtttgccgacgatgcagaccccatcgacagcccgccgttcactgcggccgataccggcgtcggcctcaatggtgacatggtagtctggaaccgggcgaacatcctggaagtcgtcgtcaacgtcatcccgaacaccgagggcgagcgcaacctggcagtcctgctggatgccaaccgcaccgggaaagacaagtcgggcgctcgtgatgtcgtcggtttggtcgtggcgatgccggacggcagcaaaatcacctgcaccaacggcacccccatcgacggcgttctgatcaacgcggtggcaagcgtcggccgtctgaagaccaagccgtatcggttccgattcgaaaaagtgatcaaagccggtactagctgatgaagaaaattccgctgacagcagtccccaaccaggcgatctcattcaacgccggtagcagctattggaagattcgcctgtaccagaacatggacatgatgaatgccgatatcagccgcgacggcgtgatcgtttgccatggggtccgctgcttcggcgggattcctcttctccaatacagccatcagtaccgacctgactatggaaatttcgtcttcgaccgcgacgccgattggacattgttcggcgacggcatcaacctgttctatctggacggcgccgaattcgccgagtatcaggcgcttgccacgaggaaagaatgagcacatcaacgatcagaaccgggacgaacaacgatatccttttggacgacaatggaaacatggttatcctcagggatgtcgaagcgtgcgcccaggacgttcgggcggcgatgctcatgcgcaccggcgaaaacattttcgatgtgaactccggtgtgggatatttcgaatacatcttctcgccgcagaaaagctatgatgacgctcgcaaatccatcgcggatgcaattttatcctcaccggatgtgaccggcatcgagcagcttgacatcgacataaccggggaagtcttcggcgtcgatgcgaaagtcatcaccatccacgggcctgttaccacaggagtttgaaatgagcacaatccgcatccaatacgccaacggcacccaactgttcctggacggcaaaaatccgccgcccctggacccgctgccctcgttcaacccgtctgtcgaagatctggaaggcctggaccgcgaaaagaacaccgacaagggcgactcctcgccggccggtcttcccgttcccccggtaaacgtcgattccaatgtcgacaacggcggaaccatcccggctccggtatcgaccgacgctcctgcggccgaatcggccccggaaggcgccaaggaagctcctgcagcgggccaaggcgacgagaaaggcgccgaggaagccccgactaccgccccagtagaaaaggccgaggaaacgtcctcgccggccgctgaagaggcagccccggctcccgccaaggccacctctcgcaaaaccaccagcaagtaaggactcgacatgatcaacgtcagcggcttcggcacaggaattgtaatagtttccgcctcatcgttcccgatggggttttccttgtcgaagttcgctgatgatgagagtccgatatcctccaaagagctggagccgttcggatatgagatgctttatgacggcggcctattcgcctttgacaaggctgctcctctggaagtatctgtatccgtcatcgcagggagcgaggatgatattaaccttcgcatccttctcaattccaaaaagggatcattcagatttcttccgggaatcatcccggacatgacgactctcgtggccactcttcctgatggcggccgcaccgttctgtccaatgggaccatcctcaagggtccggccatcgacaccatacagaacaccggacggcgcaaaggcaacacgtacactttcgttttcggtagctatcttggcgcccagactgcgcgtcaagccatttctaacgttatccaatcggtactggaggtggtctgatgttagggattttcaccagcctcctaagttcgcgatctttttcgattgtggaccaaaacacaaaccagctagttgctgcggatttgaggataagccgggtcaacacccggttttcttctgtagggcaacgccacatgctggaagatggtacgaccaagatggattccagaacgatccaccctatggaaatcatcgtcgaagtattttgcccttcaattgatgtcgtcgatcagatcaatcaattgctcctggatcgcgacacactgtacaaagtcatcactcgcggcatggtattcgaacggatggtgtgtaccagcgaagcgctcaatcagactccggatatgatatcggcgactcctgcgcggctgacattctcccaagttctcgtccagaatcccaagcctataatgttcagaaatgcaggggactcttctatgatcgaccgagggctggccctagctgaagacgtggttggctcggccggcgatctgttcgactacgcagtaaacggcgtccagaacgccgcagacttgttctgaggtgccaattgaactctttcctcaagtctattctcaacacgcctactctcaccatacgcgatgatgtcaccaaacttcctgtctggaagagtcttcaagtcaagaaagtggaaatttactcgccggcttccgtagtgtcgaaacctctggcgacgaaagaccagacggaagctcaggtgtataccgaagcgctggacgttgatgtaaagaacgggaagatcattcagccagtgcgtcttcgcatcaatgccatctgtccagacttgtccacagttgaaagtatcatgaatgctttcaatgacaatacctcgactttcgccatcacttccaagtcgatattggctgataaaatggccatcatgacgctcgatgtagatcaatctcctgacatgctaaatgcggctgagatcaacatggaattcgagcaggttgagcctccagtattgaatgaatttgatccggctttccctcaagatcgcccaacttatggcgtgcagattcagtccctttccgatgcaaatttgctagacttgggagccaccggcgattcgatatcttcggccgcaaaatcgctatataatcgcgtgaccagttatttctgaggatgtatcatgcttgaaatcaaccttcccgatggccgccaaactcgcgtacaaatcgaggcgtggtcggcattggacggctgggaactccagcgccgtttcgtcgaattcgccgtcagccaagattccgatctccgccgctctttcaccatggaaatccttggctatgctaaagtgatgcttggcgacgacgacaccggtattccgctgaccactgcggcggtcatcaacaaccatctcggccactggaagaacgtggaactggttttcaattctgttctcaagcacaacggcatcgacccggccacgcacgccgaccggccggactattgggaacaagtcggatcgcagatggccatcgcatttctggccgaggcgtccaagctcattggtccagcaatgaagatcgccgaaggactcgccaacaagccggagtgattcatgtctagtgatttggatgaattcatacttcggtatgaggccgatactgccagagccgagcgcaatctggagcgcctccagaaccagatcaggcgcgtgaacagcgcatcaacgagtggccttcaggatttgcgccacttcgcagacggcgcggccactgaactcggccgagtcgttccgcagatcgattctgtgacgagcgcgattcgcgggatgaacgcgcaactggcgataggcgccactggcgtggccctggtcgcggccggcgtcaaggcgttcatgaacaccagggaccagtacaaccagcaacgcatccaggcgatggatatcggcatcgccccggcgcgactggaagagtaccagcggaagtttgctcgccagtctggaggcacgatcagccgcgagcagggcgcggagatgaccaaaaatctggcagacactttccggcgtgcttatcgcgacatcgggcgagtcggcccggaagcgcgaattctgcgtatggccggcgttgatgtcgggagcttccaaaagggcatgcggccgctcaacgacatcattactgatctggccacgaaaatggccaagctgaagccggacgaaatttctgcctacgctgatgcccttggcgtctcgcgagactacctgagcaccctggctaagatcggcccagccatgggaaaagtcaccgagatgacgactgcggaactccagtccagggtccagggcgagtccaacattcagaaattcaacgatgctctggcgaatctcaaccagacgttcaccaccctggaaaaccgagtcggcgaaaagctcgcgcctgcgttcaccaagctgatcgaaatcatcgacaagatagtccaggctattcccaatgaagtggaagaattcgccaaggacaccaaagcccgctgggatgacggcatcaccggaaaggctacggttggtggtgatatcctttcccttctcagccctggcgccctgctaggccgcctggcctcctggggaactcggcgtgggatggaagaggcaggcctgatcgacaagtcgaaggttcccggcgctcaaaccagcgaagacctggccaagaaacaagaagaccaggacaaggctactaagtccatgaaagagctagagaagctggctgaccagaccacgaagtctaccaatgattttgcggtggcgatcaacatgttcagtggcgcggtatcgtcgttcgcgaatgccgttgacgagcgccaagcttgggcagcctgggcgggggaaattggtcgggcggtcggtatgggaagcaccgcgccgacttcgcgggccaccggcgtctatccgcacgcgatctatgatcagtcgaagagtggcgcggccggacaggtcttcggcgagccaatcggcgcccagtctcttcgcaatcgcatgttctcgccgcagcgcaaggccgaaccggtcaccgttccatcgtacatcaacgatatcatcaaagatgcttcgaagatgtacaacattcctgagctggacatcaagaaactcatatacaccgaaagccgattcaacgccagggccaccagcgaagccggagctaaaggcctcatgcagctgatgccggaaatcgccaaggcgtatggtatcactgacgtatatgaccctcgccagaacatcctcggcggaacgcgcctactgcgggaaaacctagatcgagcaaatggcgacatgcggttggccttgacctactatcatggcggactcgatccgaagaactggggcccaaggactcgcgcatatccgggtttggtaatgagcgctccaatcgagctgatggaagaggctcagcgcaagcagaaggctgcggccatgaccgtcgccaacgagacgttcgccccggaaggcggcgacatggacattcgtccctatgatggcgggcgactggaaaccccagaccagggcaagaaggaagatgagcgccgcgaagcccgtcgatatgacgacagggttgtgcgaccggaaattcgcatcatcgaccgcatgccagaccgcagcgacggcgaaatcctcaagatgtctagacgtcaagaggccgaccgggcagattctggattccggaagttcccgaatcaagttcgcggcgagaccaagcagaacatccaggctcaactcactgcaggagccatcgcgcaagtgatcggcgttaaccccaaccagatcatgcgccgcgaaatcagccgttctgacttgctgttcggatacaaccaagccatcctgggcaagcaacaggagatcaaggccgccgcgaccgaagccaacaacgtattcctgtctccagccaagcttgccgaagccacggccaaggtgaacgccgcatcgcgagaaatggatattctccggacgtacggcgagcaacttctgaagagcgctccagagcgcggccaggagctgaccatcggtcgaatcgacatgttggtcaacgtcaccggcgcgaattctccggaagaggctcgcgagatattcagcaagcagactgcagaccagcttactacggcaatccaggacgctcaaaacgattccgcaactaagatactctactgatgaaaaagagaattctgcgagtaacattcaacatgccttatggacccgaagtcatccgcgaagatctggatgttcgggtccggattatgaaggctgcgttgcggattcagaaccgggcgacgatggaaattttcggcctcaccactcagctgcgcgagtctcttctgtcgcagttcaccgcatggaagcaccggcaacgtcaagtgggcagggaagatgaattgatgatcaaggtgtcggtggaagccggctactctgaccaggggcgcgagcaagtttccagagtctttgtcggcgaagttgccattgtcgatatcatttctccgccacctgacattggaatccgcatccagtgctataccaggcaaatcgacaggacgaagaccattcggaatatgccgcccgccaacacgacgtttgtcaagttcgtcgaatggggcgcgaacgaaatgggattgaacttcatctgcgataccagttacaacgatcaagttctgaagaatccgggccggtctatcactgtcgcgtcggcaatcctggcgtcgattcaggacatgtacatgccggacgtggccgcgttcgtcgatgatgacatattgatcgtgaaggaccgcgataaagtcattcgtccggatgaggtgaccaacgtcaactcgtttgttggaattccatcttggtctgaatggggcgtggaattccagtgtctgtttgagccatcgattcgtgtggctggaggtgtagcggtcgaatctctcatgaatccaagcgtcaatggtaactatgtaataaccgctctggaatacgatttggcaagtcgggatcggccgttctatatcaaagtcatggggagtccagcagcataatggccagggaaatcaaatcgttcaacatgttcggagttcactatacttcgcggcaattctctgctgtcgatgggctcagcatgatgtcggaaattcagagcgtgccgccagaagaattgctcaagggcaccgatgtattggcgcatccggaagaccatccggaaggcatctggcttccattgaccgctgcgaacatcaatctttatgtcgttgatcgagcgaaagtaatagctcccgtgcaagttcttgcacttctgtcagaagtggtaatcgactggaactttggttttctcaaagactggaccggagtcaagattccatctagatttgtcgaagatatcaaaagtgtgaagacggcacattcaccttctgtagttgcgagtttggtagcgaacggttcggcctctatgcgcgagttggaagagtattattcgactcaagatgcctttaagatgatagacatcatgacggcgaagagcgtgaacgaggccttggcgtccgaagcatcgcacaacagaatcaaaaagggataattcctaaccgggcctgggaaggctatactagacctgccaaatcagaggctttcccatgtccaatatttctctaacatccgcaaaagctcccgacaggacgcgactgatcgccgctcttgacgctcggtcgcggcgggatgctctagatttcgaagtaatgataccggctcaggttgttcaatatgacagggccgagaatatcgcgacgattcaacctctcatcacctgggttgacacggaacacaacgccgtccaaaggcatcagctggttgatattcctgtaatttccatgggggctggcggcttccacataagtttcccgattcagcaaggtgacataggctggatttatgcggccgaccgcgacacgtctcagttcctggagtcgttgtcaatgtcgaagccgaacaccggccgcatccataagttcgagcatggcctgttcatcccggacgtattccgtcgatacaccatcaattccgaagattcggccgctatggtcatacaatcgacttccggagcgaccagaatttcgattcgcggcgataacatcaagataactgcgccgtcgaacgttactgtggatactccgcaggcgaacttcactggcaatgtgactatcgctaacaccctggtcgtaaacggcatcaacgtgaacaaccacggccacctcgaaaacaacccgcctgatgcccggacgaaaggcggcatgattgcttaaggagattttcatggcaagttttgatttttctgatttaacagcggggggggttgtaatggctaactacaactacatcgtcgatactggtgtgatagtcgcagataccgccgacgttctgagcgacgttgaggccgagttccgcgccgccctcggtgccaatatcaacttggccgcgagcactccgcagggatcgcttgtcgcggccgaggccattgcccgttccagcgtcatgcggaatgaagcgcgaattgccaataccataaacccgaacgtttcattcggaacgttcctggacgcgatctgtgccttgatggggatcgagcgtggttctgacctgtcaaccttcggctatggagttcaagtcaccggccgcagccaaacccgcatttccacagggtctagggtccagactccggccggcgcgatcttcacagtgctgagcgatgtcacgatccctgctggtggtgtcgcgaccatcgatatcaaatcgcaggagtatggcaacattcctctgccggtcgggaatctcatcatcatcgacggaacaattggctggtccggagcgaaagtcatcgcctccactcgcgtcgatccgggcagccgccaaatgagcgatgcagagttgaagaatgcccgcgttaatcgattggccatccaaggccgcaactcgactatggccatcaaggcgtatgtgagcgccgttccaaacgtcacgtcggtgaacgtaatcgagaacaacaccggcgccgttcaagtggtgaatggagtctcgtttacccttccgtatgctgtttgggtctgcgtcgccggaaacccggataaacaagcagtcgccgatgcgttgtgggccgcccataacggcggaactccatgggactatggtgcgacgaacaacggcgtcccggtggacggtccgaatggcgttcctgtgcgcgatccggcgtccggtcgaaagtatgtcgtgaagtggaccactccaatcatgtacgatggatatgttaacgtaacagtccagcaaggttcctcctctgtagctccggaagccattcagaacgcagtggtcaattacacccaggggaaagtggaaggggaagaggggctggtggtgggcgcgagcctgtcggcattcgaagtcgccggagccatcgctcgcgaaattcccggcatctacatcaaactctgccaggtagcgtgcgtcgcggctggctcgccggctccggctccgggcgatttcacttcggaatacgtcatgagcgcattcggccaggctaccatttccgttggtaacgttcgggtgactttcgtatgactctgcctgcgtacaattcggacatccaacaagctctgaaatggctccataaccaggcgcctggaatcaccggcctgatccagagaaaggcgcaatggtatgacagattcagccgccaattctgggccaactgggagcgcgacgttttcaacttgaagaccgccaacccgttcggccttatggtttggtgcatcatcctcggcacgccttcgaaagggttcggcctttatccaaagaacagttcttgggcattcggtcggctacgccagaacttcatctatagcggtacacaagtcccgccaccggcagacgcatcgcctggcggcaacttctacggtggcggcaatgccgaaattctcaacttggacgaaatcaggaaagtgcttcagctaagatatgtagcgctgatttcgaacggctcgattgcatatatcaatcgcatgcttcgttacatattcaatgatgatgagccatgggacgaggcgaccggtctgtacttctatctcatggactcaaccggcgagaatggccctgtggagaacttggcagtatatcggaaagattgggaaggtatggtgttgttgtccagttcgcccagaacgaaccacgtgctgacatcgacccctgccagcgacgccgattggccaggagtcgatccggccgcgagcggtcttccggtaacggtcgaaacggcgtccgctacggccccggacggctccgctacggtgtgcaagcttactaagccggccgggagtaccgcttacgtctccgcgccgatagatgggccgctggggtccggtagcactgtaacgttctcgttcttcgcgaaagccggctccacccgtttcattgcaattcagtcggctgccgatttccccagtcgagccgatgccgttttcgacctggattccgggaacgtgatcagcgatcagatgttggacagcagcgtggtaagcgcccgaatgattcgtctggagaatggctggtggcgttgcgttctcacgaccaagaccgtcagctcttcgttccgcgcggcttacatcgctccggcagaaaccaacttcagctggattgattcgaattccagcgcggcgattgatgtgcttatctggggcgctcagatcgaacttggtgatactccaaccggatacttggagactaccggaacgcccgtaaccatcaccgattacgttctgcagagcgcccagaccggaacggtcaagttcacacagcctcttccgaccggagtagaagcgtattggactggagactggaaaggtgggtctgcgaccgagccggccagattcgcagtaggggatgggactcaagatacattcaatctgtccagccctgcatacatcggcctacccactagtggggcgttcaagctagaatacagagttggtccggcgcttaatttgtcgccgcaattgatcaacctcataaatgaccgggcggtcggtatcatgccgacttgcgccggttgcgatgtaaaagtcattcaggagtaatgacgtgatcacacccgaactgatacccagtccgtttgctgcgcagggcgacaaagacccgatcccgcagacctcttccactggctttgccaaccttcgcgacggctacacgccggactacgaaatcagcctggcgtcgaacaacccgcaggccaaagcggtcgagcgaaagattcaaaaccaactcttcttcatcgcgacccagaatgcgcaagcctggcagcgtcaaatggcgccgccgtggtttcagggtatgcccggtgggtatgaacggaacgccgaagtcgtgcgcgtcggcaatgacggaatcatgcggcgctatcgctccatggtgaacgccaacgccagcgatcctctcagcagtaccacctgggaagagcaacccgcctggtcagttatgcgcaccaacataccgatgccggctggcggccccggtctgtcttcgggcggcgaagtcatcaccaccggccgcaacttcaatgaattgctgaacggaacctgggaatttttctctgatgcaatcgtcgtggcctctcagaacgctccggtgtatccggcgtctgccggcgcggcagcaggaatgctggaggcgaaatcttgggtgtccgggtcaaatacgttctgcgtccaacgctacactgatcgcgtcggaaacgtcgccgtgcgcgggctcaatgccggggcctggaccaactggatgtacgctgtaaacgtcatggccctccaacacggccgtgtaacctatggaaccgcggccggtccggcgaatgcctacaccttgactctcgttccgcagatccaaggtggtttggtagatggcatgatccttcgggtcaagttcaacaccatgaacaccggcgctactaccatcaacgtctccggactcggcgccaaagccatcgtcggcgcggccaacttccctctcactggcggcgaacttggccaaggactcatcgctgagcttgtcttcgacgcagcaggcgaccgctggaggattctggcaggcgcgccgcgcatccaagtcggcaacgccgatcaggactaccaggcgccgagttggaagcaagttaaagactatgtcgcgtcccaaaagctcaccgaagtggattggacggatgtcgtcaacaagccgaacgtcgctatccaagatacaacgccgtggttcgccaatctggagctgtccgatgctcggcctttcatcgatttccacttcaacagcaatcgcgccaaagattttgactatcggctgatatctgaagcagacggatcgctggctttctattcgcggcaggggtctgctgggcctacccaggacatcctgttcaaccgaaattccgtgactttcttccagccgcgactcgatgtggcgaaaaacctcgcgtatatcgcgaactctggccccctttggcagaacaccaccgccgatcagcccggttggaaattcacctttgcacaaggcgtggacgcgaacaacaacgcggtaatcgcagtcaataccaccaatccggacggttcctatcgttcacaggtcatgcgatgggactgggcgtccacgaacgtcatattcaacaaccgtccgctcttcgccggtcaatacaccccttgggattctgggaacttcgatccttccaccaagttgacggtgagtgccacgaaccaaatcgccggcccaaccgggattcggaataccaacggcaacaccggcaacatgaacacctggggttccggctccacgacggcatcctatggcaatgctgccattcaaatcttcggaaaagggggcggtgagcctgccgcgatctatttcgacaactcccagaccggatggtatctgggcatggacaaggatggacagctcaagcgggccggctggtcgctcggcaataactcctatgtgatcaccgacgaatccaacatccgaacccatatcaacacaatggctgcgcgcccgatttgggggaatgtcgagttctggggtccgtggaacttcaatcctaacacgaagttgacgcttggctcattcaatgacagccagcataccaggatggtcaacagcgccgcgaaggatgtaggaattgcgtccatgacgagctatgcggacgcggctatgtcgttcttcaactatgaggcttcgacgccgaccgggaatcgtgctgctgtaatttcgtttgttcgtaacggggcacgaggcgttctgttcggcctggacacggacaacaagctgaaatggggcggctattctctaggtgccgtcgcgttcgagattgccgactccaacaacctcatgagcctgtggtcatcccacgctgccgcgccgaactggaacgggcagaccatctggaggtcgggaaacttcaacccagacaccaaggcgactttggcagctcgcaatacgacgtcatcccctacaatattcagttatggggcgtccggaatcgcatcaaccggacaggtcggtgcgttggttgtggaaaacaacagcgtcaccaataccgcagccgccatcacgttccattcgccgcagaaatatcaggtcaacttcggcctggatgcggacaacgtggtaaagatcggtggcggcacaatgggcggcgtagcatatcccatcatccactccggcaactacaacaactacatcaaccaggcgctggttcaggtaggtcttggcggcgtcggttcctacgcgatccttgccgtattggacacctccgcgccggcagcgtccattgcccccggaacgatcatggacagttccaagctgttctattcgtcctgcgattcgacctatcgcagcagcgcccgtccgacgggcacctggcgctgcatggggtatgtgtataaccgagactccaccaacggcgactcggcatccctattccagcgggtaacgtaacatgaaatggacgcggatcagaaacccacgttggctggacgcggtaaacatccacgccatggttactttcgaggggattggagaagttcctttcaccgccaatccgcacgacgtggaggcccacggaagggtcattcacgctgcgatcctgtccggggcgcacggacctatcgccccagtagactcgacgcgggagcaggccttgcaggacgctatacgagacagggaaaagcgggctatccttcgtgatacccgctgggccatagatcgtcacgacgagcagaggcggctgggtatcgaaaccacggacggacctgagttgatagcagcccttgttcactggaggcagcagattcgcgattggaatagcggggatcggccgcgacttcccatggctttgaaaacaatgttcaaaaatcaggagtactgatgaaaataacgaaggatattttgatcacaggaaccggatgcaccacggatcgggcgatcaagtggctggatgacgtccaggcggccatggataaattccacatcgagtcgccgcgagccattgcggcctacctcgccaacatcggcgtcgagtccggtggcctggtaagtctggtggagaatctcaactacagcgctcaaggattggccaacacctggcctcgccggtacgcagtagacccgcgagtccgcccgtatgtcccgaacgctctggcgaaccgcctggcccgtaacccggtcgccatcgccaacaacgtgtacgcggatcgcatgggtaatggatgcgagcaggatggcgatggttggaagtatcgcgggcgcggcttgatccagctgaccgggaagtcgaactattccctgttcgccgaagactccggcatggacgttctggagaaaccggagcttctggaaactccggccggcgcgtcgatgtcttctgcctggttcttctggcgcaatcgctgtatccccatggcggagtccaacaacttctccctggtcgtgaaaaccatcaacggcgccgcgcctaacgatgcgaaccacggccagctccggatcaaccgctatatgaagaccatcgccgcgatcaatcaaggctcctgatattccacccaaagaaaaggccgcttattcagcggcctttttgctttccggctttgcctcttcaaccatcttggcctcaaccggcgcggcggactcctcctgagtgatcgagtccacatagttccctagtgaactcagaacgccgattaacagcgctcttaccaccttgtccttgactgtctcgcctatgatcttggtcagaacggatattaactcttcccggagtctggggctgattcttggccgaaagcgcttgcgatgctctttgcgtttcatgtttagtcctctgtctgcggtcttctcctcaccccgataattgcttggggatgctttgtgttaatcggaagggtcgggcgctattataacccgtcgaaaatgctagcgcttaactgtttaacgatacgcaccgcgatattaaatcgccttctttctggccgaggaactctggcggccgggtccggtctaaggcctaatttgtcgacattaaaacgagaaaacccggatcgcctgtagggtaaggcgtccgggtttatttcgatctagtgtccgctagaatcagtggcttccgccccatccgtccagccagcaatcgaagacggcgtgtctcggcttgtccttggcgccatgggagaagtgcttgaaccggatgacctggcccttgagatactccctgtcattccagcggcgctgtttctcgtcgtgggtcaggctggaggccgacacgttgaaggtgactccaggccacagacgttcgttgcggcagacgaacgcgccgaccattccggacggcgacaggttttccgcatgactggagcgggccgtgttaccaagttcattgatgaacgcttcattgttgttgtgcatcaactcttccacgtcgatgatctcagcttcatcgtagtcgtagcgcttgactttcacacagtgcccttccttggcagtcgagcgaccgaacttgtacagtccatcagcgcgcttgcccatggaaccttcgaatcccagcattgtgtggcggcgctcgacttcgctgaactgttcgatggaagtcaccagttcctgctcgaccaggtgaatcctctcatagccgaggcagtgacgaaggaagttcacgcgctcggccgccctggctaggcgatcttcggtcggtgcgcgaggatcggtgaaatcatcgaacacatggaaagaccagtccggctcaccgctgtggcggcgaagatcgccggacgacttctggaaaactttcgggtcgctgatgtcgccgcagaccagctcgccgtccaggccgttgaacagtttatcgctgaggtattcgtagatcgattgattgttctgccgcttgagttgacgagtcagcgcctcgccttcgaatacgaagcagcgaaatccatcgatcttcggcgagaaatacatcggcagttggccttccagcagcttcgggtcaaaattcgatgcgagcatgggtttcatacagttctccagaaaagaagcccggcgaaccgggctaaatggcggtaagccggctcagatggtttcgttggcgtggttcagttcggccatgatcgacgcatagcgctcgtccgactccttgatgaacacgccgtcgtacatgacgcctttgcgatccttgatggtgtcgtaggccgcagcgtagcaggcgagcatggtagtgtcgtgctcttccgtcacatcgaacagagtcatgacggccaagaccaggctcttgatggctaatccatgatttccgcgtgccagcgcgccggccagatgtccgaggtgctgcaaatcgtcgccgtaggtgggatgaacttcgacagtctcgctgaagaccgatagatgatcgaacagattttcaccgagttgcgcggccatgatcgtggcaaccaccattacgtcgccgatgccgtctttcacctcgactggtttgttataaacccaggcttcgcaaacttctgcgaactcttcgaccaacttcaggaattgatccttggccgaagagcctttgatcaggttccggtcggaaccccatttaacaaccaggtcgtgaagttcgcgactcatgatttgttcgatattcattctttcgattccttttggatttgggatttgactgcgttgattatggacgccgtgctctggcgcgatccatccttggtggtgccgaagtagaaggccattaccgacttcagctcggcgaaccaatagccgatgatggtgccgatggcgaccgaggatgtcgggtccatcagagcctcgcggccgaaggtgaagattgcgatgatgatgagaatggagcctgtcagaagagcgaatgtaatcgccgggcgaacgaaatcgttctgctgcgcggcaagcctcctcgccgaatccctgtctgccgcctctgcggcgaactggctaagctcggcctggagctgattctgctcagactgaaggcgattttgttcggcttgaatcgcaagctcctggagacgaacgcgctcggcgctctggagttcggcgagacgcgctagagcttccggattcgcgtctagagcgctagcgaccgaggctgggtcggccttcgaccccagcgccgtcgcgacgatagcgccaacggcggcgcctgcaggcccacccaggagcgaccccagagccggggcagccgcgccgatcttactacctatgtctttccagtccatttcgactcctcaaaagaaaggcgccattacagcgcctttctccggccggtgatttagaactcttcggcttcggtcgcatcgccgacgccaccggcgtcactgcgaggctgttcctgcttgctgtagtccaccttcacctcgccgccgacgaacgacttgtacaggtcggccgcagccttgaagtgatccgggttcttcaccaggccttccagctcgaactggacgccggaccagctgcccttgtcgttcgacatgcctacggtggtcatacggaccaggttggcgaaagtcggcggggtgcgcaggccctgcggggtctgaacctttttctgggacagcgcggtcatgagcttcttcgaggccttgatctgcgaagacgacagggagatcagagcctggccgaaatcgccggtgtccggatcgatgacgatgacgtaatgaccacgggtgtcggcgaagtagtcagacttcttgtcgctgaccgaaccgtcttcgttcggcgcgtacaggcgtccttcgacttccttcaccttggtcgggtctttcatcatttccttgaagtcttcgacgctgatggcgcccttgaaaccgccctcggcctcgcgaccggcccagcggatgaactcgcggcggtacgcggccggaatgatcagcaggccggtcttgccgtcgtaaaccttgccggtgacggtgttcaggaacatgccggccttcgcgccttcgatgtacttcgggtcatcttcatcgacctggggagacatcttctgcaaaacctggatgaaggggatggcataggactcggcatcggcgccttcgaagcctgcgccgtcatacgatcccaaatccatgaagtcgggaacttcggtagtcgcgacggcgccgccgccaccggtggcaactgctacggccttggtttcttcggttgcttcggaagtttcggttttcttaccagccatgttaggctccttgtttgtcgaatttcagttatcgctaactgtgggtttataataacggaagttgcaactaagtaaagcaaattacatatcaagatttgctctttttcaccttcggtttcgtgatcttggcctctttgtattcgtgtacgccgatgaaatctggcaactcttcgcccttctccaggtactcgcgaccgaacgcctggagggtctggtaatggacatcgcggttgatggtggcgtcatagccggcttcgatgatggcctcggccgccttcttcgcatcttccatttcgccgcgaccgaattcggccagaaccttggtcttgatgatgccgtcgttgtcggtgtcttccagccacttccagaacttcgatttgttctcttccttgacggaaatgatggccttcggctctacttttaccgtgcgaccgtccgccagagtagtggtcttctgaccaagctcctccaacagctccggaatggtgttacgcttgagggtcttcagctcctcttccttttcggccagcgccttttgaagatcgaggatttcgccgtccagctgcgaagccttgtccaccaagttcagcagtcgatggccgatgtcggtggcttcaaccgccatttcatccatgacgccaaaatagtcgatttcgcccggcgcgttctctttcaagtattccgggatttcaagctcttgctctttcatgtccgcctccaacttagtgatgttcccttacttgaaccaagtattgagtagatattatgccgcatcttccttgatacggctactgatttacatattaaatttcgtcgcgagtgctaacgtcagcctcaaacacgccatcaacgacataacttgccaggttgcgtttccactccaagctgacctggattttctcgtcgatggaatccagacagatcaggtcgaagtacagaaccgagttgacggtgccgattcgatgatttcggtcttcggactgcatccgcagctcgttgtcttcgtcggtcgtgtagtagatggccacgtctgcggctgtgagcgtgatcccgatccctgctgcggccgggttgcccaggaacacctggacgcgcttggcctggaaatcgtcgatcaatttttcccgctcggcctctttggtctcgccgtagtaggtaccaaacgaaatcccttgggcctccaaatacgccttgatctggtcgatttcctgaatgcgcatggcccagacgatgatggaacgctccgggtcttcctccagcagaccctccagaagatcagtaaacacggcgaatcgcgggttgtcttcgggcggcaggatcaccggctcaccatagacgttgatatagccggacgccacttgcttgagcttcgagcgcgctgccgcagcatcgaaagatacatcaagcatgaaatcttcgttctggagaacaaagtggtagtcttcttccacgcgctggtaaacctttcgttgctcaggcgacatttcgaagtatatgcgcttgtacactttgtccggcaggaatgggagtgcttctttcttcgttacacggaaactgtgcggctcgatcagggaccgcagcttgtccaggttccggaataccgggcgcccataatcgtctttttcgacgagctggggcggcatggtactcttaccgtccaattttcgcatgatggctaccattcgcggatcgtcactgggaaccagaacggaaaattcagccacgaacgcccgataggatttcgtccccagaataccatcacgcaggaattgaaactgcatgaacaaatcggtcggcgctcgcgtcagaggcgtgccagaaaggatgcgccgtgccacggccttctcgcccaacttaacaatctttttcgcgcgtttggcttgtgggtttttgatcctcgtggattcgtccacaattgcgcagactttgaacgtcttgaggaatcgctccacttcgtcatagccagcctgatggttgatggcatcgacgtttatggcaaaaacccgaagaactttttcatctgcgaacgtctcggcatacagacgatccaagcgcgccctggcctttttggaagtcggtcggccgcgccaatccacgctcagggtcttgatggcgacgtgtgtcggaatttctcgaagaatccagttggtatggacaccctttggggcgacgatgagcaatgcgtccacccggccctgtaggaagaggcgaacggcatcggccaaagtcgtccaagtcttcccggtgccctgctccatcaggtatgcgaaattccgtttgttcagggacgcggccagggcattgaactggtgctgcatggcctcggtcttcatgcccttgacgggatatgttttggctttcatttgttctccagatcagcgaggaactggacgatgttgtccagtccttccgcatagctcgcaacttccaccaaatcgcggctgttaagctcgaacaaatcgagcataggattcagcagcagccaatcggttccgatcttggccaggacgaagccccggccaccccagccgatccgctcccgaagaaaagggatttgcccaggttcgaaacatctggccattgggcaggtggaagcgcgtttcggccactcctccaaagccttgaactcgacccagaactggacacctcgacgattcaggcagatcgaatcggacatgccagaccgccgcgtctccagaaaatcgatcaggattcgaccgagcgagcgctgcttaaacgcattcgcggctttcgtttcgcgatcattcatcgccattcccctcttcggaatttttctctgcttgcgctgccaactttgacttctcgcgctctgtcaatatccgcttgacggccttcacgatgaacatatcgattccgctgagcttccagcctttgatgaggaaccaagaacctgtcggcgtcccttcagcaatctgctttccgtattgcagatatttctcaggccgaattctgaaacgtatcggttgatcaacagaatcgtccacgcacatgatgtcgaggaactgcgactggcctttgtacaccgggtttttcccttggtcggccctcttcttctggcgaatcggttcgttctcatcagacaaaactttcttcaccatcttggcgatgaccaggccatcgtcgccatcgcggatgtcacgaatgttctgaatggggtttccggaagtcactccaaccagctccggattgtcataagcgtgaccccagagcgtatgagcttcgttcaagtctgcgaactgaacttcagagtttgacaaactcgcggcgactttctcccaatcctgaagggtcttcaggtggggaccggccagctctttatattgagccttcaactccttcagatcggctttcaactccttcaggtcggacttcaacaacttctccagttctttgtcttggctgattttcgccgagagaatctgggcttccagcgctgctacatcatcagacttatcctccacatcttgcgccgatatcggacaattggccagggcgatcctcgcggccttgacatcctcgcgaagacgcaggaacctctcggccttagccgggccgaagcctttggcgttcatgatgccgccgatcaagcgtccgtccgccaccacccagttgagttcggaatgctccgggtccagggccgtatattctacgccttccttggccaattcgcgaaggatagacacagtttgctggtcgtccttcgccgcccgaaggcacgcggccgcgtactccaaccgatggtatcgcttcatgtagcaggtccagtacgtcaccacggcatagctgaccgagtgcgagcggttgaagccccaggcgccgaacgttaccatttcctgccaaacacggtgagcatcgtccggggcgacgcctatggtcttggcgccctcgatgaacaattcccggcgcttgttgaaaaattcttcgccctttcgcgcagacatggctttccgaatcgccgacgtttgttcccagtcgaactgcccaatgtccttcacaatggacatgatctgttcttggtacaggaaaactccatatgttccagacagatactgctcgacctgcgggatggtataggtcacaggctcgcgaccggccacgcgctcgatatacttggttgccataccagaagacaacggacccggacgggcgagcgccgtgatgtggtcgatgttctcgaacgcggtgatgttgatcgcgtttgctaccgagcgaacggcctgcccttcgaactgaaagatgccggacattttgtcttcattgagaacgtccagaaccgccttgtcgttcaaaggcaagtcgtacaactcttgcgcggtcacgcagttcgcgtcctggatgacgcccagagttcgaaggccgagcgcatcgatcttgaggaggttcaaatattccgaatccggcttatcgagctgcgcgacgccttcagaagttaccgtacagaagtcgataacttcatcgttgcagaccaggatgcctgccgcgtggactccagagtgggatgggtgaatttcgaggtcgcccatgcaggcggacgcaatctcatacttttcacggaagtcgcggccgggttgagttttttcgaaagtgtcctccaatccttttccatatcgttcgtccgccgatgtatattcgatgatcgagtttttgatgttgtcggtatcgtggaaggggatgccgaagcgttttccgacgtgagcgataaccgacgcggccttgagtgtgttgatgttacccaacttcacaacgttccaagtgccatacttctgctggagatattcgaacactagatagcgatgggtatcggcgaagtcgatatctatatcgggaagatcggaccgggaaatgtcgataaagcgctggaagagaaggcgatgagggagcgggtcaacctcggtaatgccaaggagatagcagaccaaagagccggccgaagagccgcgagctggaccgaccagcatgtgcttcttggcaaacgcgaccagatctgccacgaccaggaagtagctgtcgaaatctttcagctgaatctgcttgatctcttcttggaatcgatcctcatactcctgggtccattccttgatgtggccgcgactgaggcggtaggcttgcccctcgcgagccagagcgacgatatcaccatccaagtggatcatcggcgccttcgccagtttcacatccgccaaccgctcgactaccgcacgagtattggtgatggcggaatcgaactcttcgcgagtcatgatatggcgtaaacggctccacaactcctcttcagtggcgatgtggcgaaggccgaccgactcccgcaccttccaggccgacgcgaaatcagcatggtcgatggacggcatgtcgttgtaagaggtaatcaccaccggctttccgaatgctctggacgtctccatagcaccgtgcgcggcgaccatcgacgcgggattgatgtcaatgtagtcgattccggccagatccagatgggcataggcctcgccagcgaatttgatgacgccgtcagcttcctggaattcttgcggagtcaatccttgattctggaccgacttggaagtcaagcggtagaacttcttggtgtccttggcgagcacccaggctttgagcttcagctctttctcgccatcgtcggcgcatttgatcgggatttccatgccgaatccgcgaggcagttctgccttggtagcggcttgttcccaacggacgtggccccatgttccatcatcgacgatggcgacgaatggggattcgatctctttggcgcgctcgatgatctccgggaatcgaccatatgcggcgccgtaggagtaaccagagcgaacgcggagctgagggaaagacatcatgcggcctccattgcttgatacgcccgatacactcccatgcgcttgcagacttcgtggagcagccgcacgtcgtcgagcgcccggtgtttctgtacatatgggccgcagtagtgctcataaagatgttgcagccgcatgcggtggccgaacaacggcgccgactcttccacagtacagatatcgagcgatgggaaattgacttcgtccaggccgaacttgccgcgagccagatcgcaggtcagcatgaacttatcgaatggcaggttgtgggcaatattcgcgtcggctctggaaaagaagtcgcgaactttctggcgctgatcgaggaaagatgggtgcttgaccaggtcttcgttcttcaacccggtgatcttggtgatgatttcctcgataacgattccaggattgcagatgaactctacttcatccaaaatcttttcgccatcagttatcacgccggcgaattcgatgattctcggctgctttctcagactcaccctttggtggaacgggagtcctgtggtctcagtatcccatacagcgaatatcatgtttgttccctcttatgtcgaaaggccggctgctttcgcgaccggcctgaggagtataccgcgacggctgaagatttacgccttctgtccgtctttcggcgtgatgcggccggagcgcatggtggcgtgaacaaacgccgaatagttgatcgagtccaaggccgaatcggcatccttgaacccgctattcgccaggcgagttagtttgcccaccatatgcatcacgaacagggcgagccgatggtcatctgcggtcttcgccaccaggccgttcgggaagaggatttccatgatcttgccatacatcagatcatttcggccataagcgctctggcggtcgcggaaaatgtctgctgcggcgtacagattgttgagaacatcttccgcgaaatcatctggatgggaatcatcttcgcccggccagacggattccatggcgaacggcgcggcgtcttcggtcgggtcttcggactgagccgtttcagcgagcggagacggggctttcgcaaacgccttgtcgagggtaggggcggagttcgggacggccgggaacggctcgcctgccatgtcgttgggcgcgctatcggggtcgctatcggccgcgacggcgaagaacggcgcatcgcaaccttccaggttcaggatgtaggagtcgatccccaggctattgtaggcatcgatgatgtcctggcgatcatcgaacgccgcgacgatcttggtaacgccttcgattttcttcaggatgtcgagcgcgactgcccgcttgaactgaggcgccggctcggtgttaccatacggccgcatgatgagttcatactcgcgatgttcggcgataccgagttcgcggtgaatcttggccctggtctggaaaaagtggttgtcggttcggccggtgatgaagaagatcatgaggtcagcatcgatagcatttctgatgcgacctacagcatgcgggttgagagcgtctttgtcgagacgagaatgatactcgtcccactgccgttccaaggcgaagctcttacggtggctatcgtcaaagacgcagccgtccaggtcgaagatgatgatgccattcttgggtttgcgttccatattcagatttccttgatggttgctttctgggtgacggttttatcgatgaacgtcccatcagaagtgagtcggaaaacttcgcagttctgagtgaaatcgaacgattgtacgttcatcgtgatgcgaatggattcgctgccgttggtcacttcgacttgtgcgatgtcgccaactttctcgacaatgatcttcattttacatggacttcccgttgatggccacaggattggcctcttgcttttccgatccccagaaatcgcggcgcagagcttcctgctcggccgagcgatccatccaggggcgatagaacttacactggtagataggttccaacggaacatcaaccacaggaatctggccgggcttcgtctccagagcggacatgaaccggtcataggatttctgctggatttcctcttcattcatcaccttcgacccatagcgcgggaaagcgcaggagccggtggcgacgcagtgcggctggagcaggctgtcgaacatcggatagacttccagaaccagccggcgcatctcgcggaacacttcctggtattcaccctgggtccgaacacacaggcgaaccttcgccatgtcactgagagtccgcagattgaacttggccgcgatcttcgtttccatgttggaaggaatgattgcacgagcgtcctggagcgacgcgccggcctccagcagcttctggtaactggtctgcgcgtcggctatggcatcgtgccacaggcggttcagctcttcgcgggcgtggtaggtcgggtcaggctcgccatttaccgtagccggttcgtcgaattcccagcggaatgcttccggctgaacgacggcgctgatttccagagcgcgactggtttcctgctggtaagccccggtgcgagtccgaacgagttgatgggtgaaattcttgctgactccctcgatctggaaaatgaagtccacgaactcgaacggcgagcggatggtgtccagcatgtacttccagtggtcgagcttttcggcctcggtcatggtcgccgggtcttggccgcgcatgcgggtggacttcgttcccaagagcaattcccaggagttctgggtgtaactgatcagagaaattttcatcagaaatcttccggaattggcgtgaagtggaatttttcggtgagagccagggccaggccttgggcttgctccctgttaaggccatactgctctatctcttccttgagcagttcgcaggcctccagacgcgcctcatactcttcagcctcatggacggatgagaagacggctccatcagaggttcggtacacaagttcgatcgccatggtgacctcagtagcagcggatgatttcggcgcggatgtcgcgacggtcgaggtagtgctcacgaatcttgtcgcgggcacgctcggcctcttcgcggctgccgaacgacaggttgaacgacgtgaaaggcttatcgtccagttcagcgccggtctgatacaggattcctaccagaacgaaagacggcgcggtcttcggctgctggtcgatttgcatttcgaggaaagacatgggaacctcttcaggatggtctggtgagtacatcaatagcgctcctgctgagcagctacggtttccggctcgtagatgagcatgtcaacgatttccggacactggcctttcacccagtcgatggccgcgaccagtgtggtggaagcggagaaagagaaagtgcggtaagactcatggatgtacggagaccccatagaatcgcgctcggtggtgcgcgaaatgactgctttgatattaatgatccggcccatcttcggcctccactttagcgattatatctgacaggcttaacttctcgccattcaggaaataactggcgcgaaacttcctgtcgccctctggcccgacgattcgggccgttatggtgagactcccgccgccaagggcatcggtgtcccggaagaaagctagcagcgcccgcttgagcgctgcttcgcgaggatcgacggccattaacctaccacgttccagccgtgctgagcgcaccacacggcaccggcccctgcaggaagactattcagaaggaacacaggagtcaggcggccatcctcggtcatatggacgaagtagcgggcgccttcaccgagccactcggccttggcgatggcacgttcgagattggccttggtggcgtaggtcttggtgtggttcttgtcggtggagaaggttacttcgcgggccatttgtcgattccttttggttgaagggtttcgcgtttcgatgagggaatattacgcccacctgatccagaagtaaagcacttttgtaaattacttcacgaacattttcttggccttctgataagacgaagaagtcatcaggcgctcgatgacgtccatgtcagaaaccagatcgtccagaaggacgttgcgccaggtagcgaaccggccgagcgagaagatgccagcctcatgggtgagattccagatcatggattcgcgctcgtcgcgaccgagcggaatgattttgcctttggtctggatggtcggctcgccatcttcgatgagatgccttttcctgatgccgaaggccgcgcaaacgtaatccatatcccagtcgctgtcccattcgatggtttcgattttaccatctaaagtctccactatccccttagtgatggattcgacgatcagagtatcaccggtgatggacgctcgaaacgttcccactttaggaccagggaaatacacggtctggaagacatcacaggggatggaaagcttgtatcgactcacgatgatggaggtgccttcaccgaatgacgggtcgattcccaggtccagccctgccgcagccagattggcgcggaacggcgcggtgctgatgatattcacgtggtcatcttgccggcgaagatactggaagaaagaggcgtcgaaaggacggctccaagtgatacgattcgcaagcttagccaccagctgctcatagtagtcggccggtgcgatccaacgcttttcagtcgccagattccagatggaccggtccgacaggccgcccgttactttcctggagtacatgttgcagtggtcgatacgcggctgggaaatgaactcgccgtcgatgtagatagccttgtgtacggtgacttcgcggaatgggatgccggtgagctggccgatgactggcgagcggaatcgcagaagtgcgttgtggcgctccttattctccggcgtcgccgcgtcgatgatttgggcttgaggaaagcgatgcgcggcgatcagtccggcgagtccggcgcccacgatgataactttctgatcaggaatcatgatgtgtcccttatgagtgtacaaaacttgagaggataaaaaagggacccattttcatgagtcccttgaagagctagacgattcaggctcagaggagcggcggcttactcctcctcaccttcggaaccatcggcgccctgatcttcaccgtcgtgctcctggccttcgtcatcggccttctcgtcatcgccctggccagcttcgtcttccttcgaagcgatggcaaccagatcgacccagcccatgatttccagcttgctcaggtagctgcggacagaggtgccatagagcagatgggccaccttctcgccgaaggcttcgatttcgaccggctcaccaacggtgcagtgctcgttgatgtaagcgaacaccttgccgcgagtcgagaaggcctgcggagtgccatggccgtcgccggtcgggatgaagtgggtggcgcgggggcgacgcgagccattggacttcaggtcttcgcggcgggcttcggccttcttacggcgctcttcctgctcttccttgcggcgctgcttctcggcttcgcgctcggccttcttctgctcggccaggcgcttgcgctcttcttcgcgagcgactttctgagcttcctgggcggccttcttctcttcggcctttttggcgcgctcggcttccttctcggccttcttctgctcgcgctcggcttccttggccttcgccttctcggcctgctcggcttccttcaacttggccttttcagcacgctcggcctccttggcggcggccttttccttcgccttctcggcgcgctctgcttccttcttctcacgctcggccttgcgcttctcttcgcgctcggctttcttctgttcggcttccttggccttggcctcggccttctcggcgcgctcgcgctctttacgttgacgctcggcggccttctcggccttgcgcagggcggctgcttgttccttggtcagctcttcgccttgggtctgttcgttctggtccatgttcttactccaggaatgtttgaaatgatggcttattggcctgtgagaggattatctctaaactaattgaagaagggaatacctttcgcctgaacttctctaaatattttttctttcgggaaagtccagactccagggaacttatttatgttagcgaagtttttaactcttacgcaaaaacaacaagtattcaattgcgcgagttatcccagtatacatcaactgactataagggatggacggcaagttttcttccaacatggcgacccgtttccattccgatccctgcgacttatggaacgtcatcgcccagccgaagtcgaatccgccaatggccttctgtgcctccagccgcacatcttcctcaaccgaaaagctaagaggattgaacttcacccagcgctcatagttcgtgccgataatgcgaactttggcaaacagcatttcatccggctcgtcatcatcttcttggccttcggggaccggcttgaagtccagcagaattgcttgctcgccgttcatgatgccatattcgtgctggttcccggtgcacaccagcttctcgccgatttccggttgtacacccttgtagccgaggatgcggcgcgccctggcgttcaaacggcggcgagtattgttgtaggcgcaaaggatcacgccatcatcgtccaagaacgtccgcatttcgtcgtccgacatgtcgaatccggcccgtaccaggatgtcgtcatactcgcggcagggtaggcgcttgccctggcggacgaacatcgacgcccgaacgatattgccggcgttgcgctcgatttcggtcatgatggtgtcacagctgttctcatggaaaatctggacgccgcgcacaggaggaacttggccaaagtcaccaatctccagaaccggaatccggtgcgacagcaagcgctcttcatcccactcgccgatcatggacgactcgtcgagcacgaccaacttcggcttctcgtcgagcgagtctttgttggcaaacatgatttcgccgtcttcatcttcaccaatcggccgatagatgaagctgtggagggtccgggcattggtgcagcccttctcgcgaagccgcgctgctgctttcccggttggcgcgacgaagactgtccagtccatcgagcagcaaagttcggcgattatcttcgcgatggaagtcttaccagttccggcgaaaccagcgagtcgatagacctggcggcggtgcgctcgatcacaccaaccgcgataccagtccacaacggaattgatcgcgtcgatctgctgactgtttggtcgaaagccgaatcgctcttcgatctgatcgacggtgaagttagatgctgacatatttgcgttctccaacgctaggtttaattgaatcgaggctcagtttaagcacgccgtccacagaccagccagtatcacgacgatacttgcggccttgcggatcgacatagaagttcttcgtgcgccgcagcagaacataatgccaagcagctccaagcgcgtggacccttcctttatacgggaaagccttcgcctgctctgcggcctctttggccccagggagccagcggacggtcgaaaggaccaggaccgcacccttaatagcctgggaagcggcgcggccgtcctttgggctatagcgatgtctatcgaagtctacccagtggtggttgccgcgccggagcttgaccgttcgggatcgcccctcgaacaccacagtgccttcgtgagttaaaatatgttccgccatcgaatgttcctttataacgtacagttatgctttacctctgcgcaggaagagtatactatcagctgactcgccaaagcgagcgaatttaatccaactttacttcggcaggaaagtggccgatactagcgccgccgcctgtactgccctccaaaacagaggatacattaaatgcaagaatgcaagatttaccgcgaccaactcccggtcggtaacccgaatcccaatgtcgacaagacccgcgacccgaaccttaagcccggtttcctgcgtcgcagtcgcgagctggacccggcgctggccgttcgcatccgtcgcgagctgatccacgccgaagcatccgacttggccaaggccggatgggtcaattcacagtccagcctttatggatcgaaagccttcccgcgccattccgtcgttcgcgtgaccggagttccggaagatggagctttcatcggcatgctgatcggcttcatcgagcatcgcgagcatggcgaatgggcggtcatggaagccggaacgaaagaaggcggcgccgtcattattccagtcgatcacatcatgcgagcgtcattcgccgaagccgaagagttctctgaaaagtgggagcggaacctgggatggcgtctgctgcgccagctccgcgagtgcggcgcccttgccgggactgaagacgagtttctgcggcggatcatcaatcgatacgttcgtgatcgcacgatcctcgatcaccacaaagtcggcgcggacaaaatctacactgatgcagtgctcaaaagcatcggcgaaacatggccgaagattccttcggggaaattcgtcggacaccgagtcgcgcagctcctgatcggccacaagctaggtcgagcggggaccatcctgaatgacctggtggacttcttggagaagttcgcggccgggcgcgataaagttctcaacatcgccatctgtaattgaggtgaatggcatgtataataaaccgactttgaatcaccaccatcaaaccgcattgttatatctgtataacaatcccgatcagccggccttcacagaccccaacaatcaggcgctaaatgaacttcggcagatggggtatgtaaaggcgaaaaaattcgaaaactgggcaggcaccggccatcttaggatggaatggacgctcaccaaggccgggattgagcgcgtcgaagtcgggtttctggggaagtgcgccgcttgcaagggaatcggtcagacgttgcttcgggggaagtgtactgtttgcaatggccgcggccaggggtggatcagcgaatggtcgcagaagccaatcgaagataatcctcaaatcgtcccaaagttcgaaaagacagatgcgaatcggctagcagacgccattgaagaaatcgcccggctggaaaaggcactggccgaatccgaaaagcgcgggagcgaactggccgcgagctattgcgatggcgtgatcggcgatgaatacggccatcctcattgtcgttataaagtggagcgcgacgccgccctggccgaagtcgagcgcctgcgagaatcaaaaggcgatccctctggcagcttcgacagatgtatgaagatgatgtacgagcgagacgagaaagcaaaacagctggaagtcgccctggccagggtcgcggagctggaaacggctctggcaccattcgcagcggttgcccaaccacagccgtgcacgcaaccacaggcccatcctgctcgctgtggttgcgagcggtaagtgccaagtaaggagttcatgtaatggaacagaagaaaccttcaccagtagatggagtcatcatgaccagcctcgacgttctccggaaagcgaagcccgaagcacaggacgagtatgccgtgtccatgttcgcaacggcgattcgccagaaactgcaacgctcccgcgataaaggccgaggcggctggatcgactgcgacgaagatattctgatcaacggattcgccgaacatgcgctgaagggaaatgagaacaacctcttggacctggcgacgttcctgatgttcatgtgggtgcgcggcatcgatgatgcgaagattcccccggcgctcgaaaaggcgcgacagcacaagatcatggaagcctggagtcgaatccatgaagatggcctaaactccgccagaaaggcgagtgctgcgcgacagttcgtggaagtgcctcgacgcaaggggcgcccggagcgacttgcatgaagcctcacgaaataagactggcacaggccgaagagttcctgcgcgaactcggccgagggattccggacgacgaacgggtaatggtcggctacgctgaagaggccacagtccaaaccgacgagaacggccgcaagctcaacgccggctggtggcccgtgccctggaaggaaggcaagtacatcaattccagatccaacgcatatgcctgtatctcgtcgtccatcaagacgcccaacccgaagaccggccagatgcgatactggcgcggcgaggcctctttcggccacggcctggcgttaatggtcgatgacatcggctccggcaaagggtccaagggcgacttcgaccgcgacgagttccgcgagcgcctagagccgaccgcgattgtggagacttcgccgaacaactaccagttctggtatttcttcaaagagccgatgtcccacatgctccagttcaaggcgctgctctattcgttcgtggaccaggtgctaaagaaaggcggcgacaacaccgtcaaggacgtcagccgttacggccggatgccattcggcttcaacaacaagcgcggggaagacggcaagttcaaatatgccgacgaaaacggcaagcccgaactcgtgcgactgttcagtgccgactattccaagcgctactcgccagaagagatcgcccaggcattcggcgtccgcatcatcatgccacagatgaagaaggtggagataaaccgcgacgattggttgtatgaccaagtatggttgaagtatgccgagcacatctgcacgaaatacaagatgggagaagcggcaggcggccaggtccagcagaatatgtccggcaaataccgcatccgctgtccctggggcgacgagcacaccaacggcgatcctttcggcgcatactttcgcggaccgatacctggcgccgagcacgaatatgtgttcggatgcgggcacgatacttgccgcaaagagcatcgacggacatgggcggccttcaccgatgaagtcgtcctgccctatatcgtagaacaattggaacgcattaaccgccgtcacatcggtgaggagtagacaatatgcaaaacgatcctggaatcctgatcacagccattggcttgctgttcctcggccttatcatcttcttcgaaggcctaaaggggtggaaaatacaagtcgcaaacttcctcgcgtcgcttctgtgctttttcttcggcctttctgctttgacgttctggttcgtcgttgcgtttgacgtattttaatcgacgaacggtacagaaattttcggatggggacggaacttattagctatgccggtttaggtaggagataatagccgtccctttcgcctcaatatgtagaggcaatgttgaatccgatcatgtaaagcagaaggcggcaaacctaacatgattatcgacgaagataatatttttgatgatggcgaatcagggtccagtgagtttgatctcacacagatagaagatgctggaatggaccctttgatgaccgccgcgagcaaggcggccgatgatgcgattgcgaggaatgagacgtaccgagcacaaaaggcggcaaagtatgccgaggcgtatgcggaaccagatctgaagaagcgagcgcgattgttgatgctcgatcaggcctttgatcttccggtcagccggctggtgaaaggaccgttcgacgacttcatcacaaagtacagctctacttcagacagcaactacctcgcggtctatgatactttgttctgtaagggcgacggaactgtcccacacccgcacttcgacgagtttcgcggccggctggtggaccatcgcggcgtggcgttcaacaacaagaccctcgatccgattgacctgatgggcgccctcgcggctgcggccttggacgatccctcgattaagaagacgattgagacttgctgcgtttgggcgcgtcgatatcgccgcaactcgctgatcgagacgtttgagaagaagataccggattgggacggcgaagagcggatcgaaacgctgctgatcgacttgttcaaaccgttcgacaccgagcttaaccggatggtgagcaagtatttctggctgagcttgtactgccgcatcaactaccctggaatctcggcgccgatttcgttggcgttgattggtgggcaggatgcggggaaatcatatttcggcttgctgatctgtaaggaactgtccggtgggcgcgatctggctccagttcagctcgacctgagccgacacgaccagacgccgttcctgcgcaacatcaccggcaactcggttattgcgaacgtcggcgaaatgtccggcttcaaaaagggcgacatggaacgcatcaaagaattcctggtgcgatcttcggatacattcgatcagaagtttgagcctggcgaaacgatcaagcgacaatggatcaccatcatggacggcaacggatacgatggactccagcgggacgactccggcaaccgtcgattctatccgatgttcgtggcacaacttcccgacgaagatggaaagccgaactgggttaagccgggcgatggtaatgaaccgttcaaggtggacttcaccgacttcggccgcaaattctggcaagcgatggctgaatgccgcgcatggatcgaagagcatggtgtcgatggatacctggatatggtgtcggaagcgaaccgcgaagtgcagaagttctctatttcggaaatggagaatgcgcgtggcgtggttcgtgatgacacgattgatatgtatctgatcaacgtgctgatcagttgcgagttcgaagagatcaagcctggcaggaattcaaagaatcctggatggagagttgacactgtagccattctgaaatggttcgatattctcgccaggaagaagccgatttctcgccacttaactccgcacctgaaggcgctggggttcgtcccgaacaagaacggcctggctggttggtgcctgcccgtcgataaggttgcgcctggatggacgaatagtatgcaaaccacgctgccgccgttcaatgatgcgcttgtgtacttgctgaggaagggcgacccggatatcactgatgaggccgccatggcaaagattcgggcagtccgggccgagcgagccaagattttgggcgaggatttctgatgtagtggttgagtgtacttggactaggccgccttcgggcggtcttttctttggtgcggcgtacattagattccggcgttgggcgggattggtagaagtgaatacagtaatttggaccagtttcgagcgcgaaaatggaaagtcgatagattttcggtatcggtttagcgtgtatggtggatggattttgcgtgaaatgttgagaaattgcgtgtttgaggtggacttttcgtgggaaatagccgcaaattactgtatagcgattctgagcggtaatttgggaagcctactgccgcgtgggttagcgggccaatttcctaatttccggtttttcgagcatgggttaaaactattctacagcgaaaatcgattgcacaatcctaatagaaaaaatctatcacggacgttacctatctttaaaattaataaaattaatagtaatttggtaatttggtaaactttagtatttgaaagcctcgcggcactaagcttgtacatatcccgtcaagtttccgattccagctagctcgcggcagggtcgccggaaacttccggacttacaatccatgggtcgcggcaacaccacggcggactaagcggcaagtgccaaaactcgacgaacggaaccggaaatttgggagcgcgacagaatagctcagctggacatatttctaacattcgatttaacactcaatccaaacactcaccaccatcgtctcccaccaacagccgactcgaccctcacccaccagcagaccgcccatataacatcctataacaccacctaacactcattcaccatcaaacccacccagacctacagcccacccacaagcagcccatagacgcgatccctggccccatagtacaatcgcgccatactcagtgtcgcggcagagcaccaggggccatccgccaaccaagccaccacgacgactccagaatcgaactccagggacgcagcaacaaatgaccgccaaattctacagccccgacgatttagtcacgccacaggaattcgcagacccgcatttcgcggcgatcaaccaaaagcgattcgacctgtacatcgacctgcgcgtccaaggttacagctcctggcgggtcttccgggccatctggggcgaggagcatatggacggtcccgcgcaggcccgcatcttcgcgatggagtccaacccgtactatcgcaagcagttcaaggccaagttgaacgcgaccaaaacgtccgatttgtggaatccaaagacggcgctccacgaactcctccagatggttcgtgaccccaccgtcaaggactccagccgtctgtcggccatcaaggaattgaacgttttggctgaaatcacgttcgtggacgagtcgggcaagaccaggatcggtcgcggcctggccgacttctacgcatcagaagccgaggctcagaccgcgaccgtcgctgccgcggccgaagccaatggctatgttccggaaggcgaagagggcgatttcccgtccccgacgccggaaccgaccgaggaagatcgcgccaaccccatttagacataaaataacatcgttctaggcccgaatcggaccgaactaaggcgacggtagcgggaaggaacgaaaaaacgattctagggcggttctaggaagtcgtagcctaaccctcagaaacgacaaagcccccggactctagctcagaatccgggggctttcttttgggcgccttattcgccagcgtcgatgatttcgaagttgtatttgacgccttcgtgctcgaaagtcatcttgccagccgccttcagctgcatgcggaagcggatgtgtttcgaagaaggcaggccgaactcgatgaatgctgcgttggtggaccggaactcgccgcgcttgcctttgacggttacagccacgccatggcgctgggtgcgtttcttggaaacttccgggtctttccaggagttggcgatggctgccgacaggtctttggtctctttggctttctccggcgcgttcttggcctcttcgcgcatcttccggatttcttccagcgcttcctcttcggtgatctcttcttccggcttcagattctcttcctcgaccttctcttcttccttcttcttggaagtgcgggttttgtaaaccttggccggggcttcctcttcctggaaggcttcttcctcggccggcagagcattcagaatggcgaggcagcgacgctcagcagtcttgcggtcagagaaacgcttgacagtcgcatcggcgttgtgggcgttgtagaaggcgaccagttctttcatttctgcgttctggatgtcgccgaaggttttgatggagttggtcattttgcgatcctctgttttggaagatttcgttcgggcttcggtttgtcgccccgttgaaagagattatgcctaggtcgatgctgcgtgtctacatttatttcatcagattgatcgcaaagccgacgaacggttgtctgatgtgaaaacaccgcagagcaggctgcggtgtttggtgggcgagggcgatggtcagaaagtcgggactgtgatcggctcgattggcgggtcgccgggcttgtcgttgtccgatgcgggagctggactggacgggatcgcgccggaccgaagcgacagagattccgactgcctgctcacctgcggagctggaggagctggaggcgcttttggcgcatgaggcgcgggcggaaccggcaggcctggaggagccatcggcgccctgggcggaaccggaagatcgggcagatcaggaagactcggcgctatgccgccaaaggaccgctgcgactgctgcgccggattgtcgccgaacatttcggcggatcggttgtccttgatgacttgacctatggccgtcttgtcgccgcccggctctgccggatcgaagcccgtgaatggagtggtaacgcagcgcatgttcggatgggtaggcagatttttcagaactatggaatctgcccagacatacggattcttgtggttgccgggtccgtggctgatgatctcggcaggaatatggccgttcatggtaagccattccgccatcccttgccgagtcagcgggcttaccacgatctgaccagtggactctttggcgatggcgaacagatcaccgaattcgaatccaatgattgtgtagttggacctggactcgccgacgaaagccagagtttcgatgttgtggtggatatcttgttcttcccagatgtaaagcatgatgcgctcctcaataaggctgcttgttgtattcgaccagagacataccggccggaactttgaagcgtatgtattcgatcatcatcgcagtgcgacgccgattcgtgccagactcgtcgttgacgaatagttcgcactggtgcggaatgactcgcgtcaccaggccttcgcctttcgagccgtagaacttgatgtaggccgcgacgcctcgcgtcatgttcagatggatcgtttcgcagagtcgctgcgcggccgacgcgctggacagatcagtcgggctgaccgtcacccagtaatgtgcgttttgttctttatcggacatcatctgccctccattgataaagcctctgccgagtagcagaggctggttttaatcgcgcttcatcaggacgactttatcatatgctcgatacttcccgcgccagtcttcccagtaatcgccggccgggcaagtcagctccagagtgatttcgtctcgccagcattcgacggaaaggacaatcgccttgttcctggctgcggaatccgctctgagcttaatcagaatttcgtcgcccgttttcagctcatcaactcttacaactttggccatgttgttctcctattgggtagcgccacgagggggccatgtcgcttcgatgtcctgtaagcagatggtgggatagtctctggccagcttgagcttgaagtccatccagtcggcaccgatgagtgccgcgacttcgatgatgacttcgttgtgcgacgccgcgctagattgcaggactatagcccgacgccgaacgggatcattgacatcgacatgagttagtatttcgtcgccgacgagcaactcacaagccatcaatattttggccatcatacactcctatgagaaggcgcggccaggacgatcccagccgcgccgatggattaacgtttgtgaaggatggacactgcgtccacgtcgaggatgctgatggtacggcgacggcgcggattgctgcgttcatggatgcgaatcaggttcgcagctggagctttgtccaccacccatacatcttccggcttctgcttccggagcaggaccgtcacttctgcgttctgggcgaggccgttgcagatgaacgtgaacttggacgagggagttctgtacatgtcgagattcctttttggacttcgggtccggcttttcagtcggtgaagagattatgcctgatcatcaccgtcgagtaaagcacttatggaccaattctccagttaaatggaaccaaagcgcggtatcgctgatcgctacgcttccacgccatggcccttcttgctcacagacttcgaaccacaagctaatttccatcctggccaggactcggccgaaggattcgagccgacgccggctggccaggatgtcgacattcacccgtcgattaatttcgtatcggcgaccgttgatgacgaagactaggcgtagggtctgtccgtccagacattcccagtatttggtttcataccgaagccagtagcgcttgccagccgggcctaccatggtcatttaatcgatgctcctggccgcgccacgcggtcctgtcggcggcgcaggatcgaaccgccccaggatgtagtcgggccggcgcgcttcctgaggacacacggcgagaaggcgccatcctgcgtccagggcatactggagttcgtccgtgcagcagtcttccttgagcagcagacggttgacgctctggagattcggaccagggatggccgagctggtgtgcgagttccaaccttcgatgccgtccaccatctgtggttggttgatgtagccttcggacctgccagccaaccggctcgcggccagctccaggcgctcaagcatgggtcgcagagcagcttccgggtcaacatcatcccagagcaggaccaggttgatgatggtgtacggatagtccttgtccagatcccaggccgaagcggtcaggcggcccaggccgatttcattacacatcacgggaacgtcgttgctccatgtggacggctcccagttccgctcaccaggattcccgattgttacgccttccaggttccccaggagaacgtggagtttgctgacgtattccgcctccagcgctttccgctcttcgtcggtctggttgtggcgatagaaggatggagggctgacttttgcatggtagagtttcatggcggttcctcggtttttgaaggcttgaacgttagagaatggtgtcgcagtatttctcgaaaggactctggcgcttcttctcgcagatcgcgcaggtgatttccaggtcgggcagctcgctgtaagtcttgccgagatacagccagcgcttgcacagactgcggccgtccaacgtgaagaagtggactttgcgagcattgccgggttgcgcccagccaccttgatcgtttttgcgcttgctcatggcgatatttcctttggatctggaatccatgccgtccgttcgctcacccaaactccgtctacatagatcaggactgacagagcagtcgcgccaaacccgaagcctacatagaatgaaccgggatcgatttccatggtggcgctattggtggcgctattgaaatcgacttcaaagtcgaacatcgaatggccactcataccatcaccatgtcgatttcattgatgaagaaatgaacatcgacaccatcggcgcgaccggtgtaacgcagccgagtcgtcccatcaatgtgggcttcctcgacgccgatgacttccaggatggtgtcgttgcagtaaagcttgacgaacatcttgatgccggagccaatcgccgcgaaggcgatctgcttgtacaggtcttgcttgatcatgctttgcgctcctgtttgcggatgtattctgccctggcttcggcttcgaacttcgcccaaagcgcgttgtcgactggaccccagggaccggtctgagtcccgtccaccggcgcgaacttgccaccggcgcgacgatacttggacagagttctcaggctgatgattttctcttcttgggaatcgagttggacgatcacttgacatacttcccgaagtagcaggtgtggagtatttccaggcctttggagatagccttcagcttgatgccgaaattcttgatcgcccagtccagatcgtggtgatagaactcgatgatgttgacgatttggccggcgctgtagtggatgacgcgaaccttgccgttccagcggaaatcttcgcggctgtttacggcctcgacttcgatggtgtcgtttctgccgatggtccatttggagctagtcatgtcgcaatcctctttttggagtgtttcgcgtttcgatgaggtaactatacctcagtcacctcatcgagtaaagcacttttgaacatattatctgaaatttctgtgagacggatccatcggcggattcagtcagtggaaccgagattccatccgttgcgggcgaaggccgagcggccagacagcttgcggcactggatggagctgccatcgtcgaaggtgtaagatacggcccagtccaggcgctcgcgacgtactgccgccgcagcgatatcttccagacgggctttgagaagctgttctgctttggtcatttcgagttcctctttggggcgattcgtgtttcgatgagatgactataactcagtcacctcatcgagtaaagcgcttctgaggaattatctgaagtttctggaggccagaaattgccgccagagccagtcaacgtgatcgttgaggtagcgctcgccatcggacgacagagccaggcagccatcctttatgccgagcttggagctggaaatccattcgaactcgaactggagagttcctgggcgagggatatgggacatgctgtacttttgcacggtcaggaatcctctaaaggtttggagttcgcgatggccgcgactttctgcatgccgatgcgatagaattcggcctcttccctggccttggccagctgctccctggcttcgcacgccagcttgtgctggttattggcattctggatgcttccgtccagactcctgtccaactcttcccgaagagccttctgcgacgctagctcggcctccaaatcctggattctgatctgaagctggcgattcctttcagaaacatcgttttcgcgagcgatggaattctgaaggcattcgtcaaggcgagtattttcgagcttcagcttgttttcgcgatccgcgtacgcagtaagacatttggtcagacgagcctgttctttcacaagatcgactttgcggagcttggccctggccagagctttgcgaagtgactccagctcaagcgggtgaagatcagcatccgggtcttcacgctcttccttcggctccatccgcaggtagtattggccggcagcgaaggccaatagggcacggtcggaattctctacggaagcggccatgaagtgggaattataacgaggcagaaacttgcccgcctctgggtcccagcacaggctatccgccataggccagaatttccggaagttatctgcgttcacgcccaccttgaccgtttccggcgcacctgaatccgtcgccacttccaggttcaggtgaatggtgccggtgatgtactggaagtacactatgctagacataacacaaactccatgagaagtcgcggccgggaccatcctggccgcgcatct